(19) United States
(12) Patent Application Publication
(10) Pub. No.: US 2024/0343666 A1
(43) Pub. Date: Oct. 17, 2024

(54) PERINAPHTHENONE COMPOUND AND USE THEREOF

(71) Applicant: INSTITUTE OF MEDICINAL BIOTECHNOLOGY, CHINESE ACADEMY OF MEDICAL SCIENCES, Beijing (CN)

(72) Inventors: Liyan YU, Beijing (CN); Shan CEN, Beijing (CN); Jianyuan ZHAO, Beijing (CN); Xu PANG, Beijing (CN); Tao ZHANG, Beijing (CN); Wancang LIU, Beijing (CN); Tao ZHANG, Beijing (CN)

(21) Appl. No.: 18/294,886

(22) PCT Filed: Sep. 6, 2022
(86) PCT No.: PCT/CN2022/117379

§ 371 Date: Feb. 2, 2024

(30) Foreign Application Priority Data
Aug. 6, 2021 (CN) 202110905681.6

(51) Int. Cl. C07C 50/34; A61K 31/122; A61K 31/202; A61K 31/222; A61K 31/341; A61K 31/351; A61K 31/357; A61K 47/55; A61P 31/16; C07C 50/38; C07C 59/52; C07C 69/145; C07D 307/12; C07D 309/24; C07D 317/12; C12N 1/14; C12R 1/66

(52) U.S. Cl. CPC C07C 50/34; A61K 31/122; A61K 31/202; A61K 31/222; A61K 31/341; A61K 31/351; A61K 31/357; A61K 47/55; A61P 31/16; C07C 50/38; C07C 59/52; C07C 69/145; C07D 307/12; C07D 309/24; C07D 317/12; C12N 1/145; C07C 2603/28; C12R 2001/66

(57) ABSTRACT

Disclosed in the present invention are a perinaphthenone compound and the use thereof. The compound can bind to E3 ubiquitin ligase tripartite motif 25 (TRIM25), thereby facilitating the recognition of TRIM25 to a pathogen and inducing proteasome-dependent ubiquitination degradation of the pathogen protein. The compound is expected to be used as a ligand for TRIM25 and have a broad application, for example, for preparing a PROTAC molecule. Therefore, the compound has good research and development value and application prospects.

### TECHNICAL FIELD

The present invention relates to the technical field of medicines, and in particular to a perinaphthenone compound and use thereof, for example, use in preparing a medicament for preventing and/or treating a disease, use as a ligand of E3 ubiquitin ligase tripartite motif 25 (TRIM25), use in regulating the ubiquitination level of a target, and use in preparing a proteolysis targeting chimera (PROTAC).

### BACKGROUND

In recent years, cell therapy, immunotherapy, gene editing technology, and the like have been rapidly developed. Proteolysis targeting chimera (PROTAC) is an emerging direction in the field of drug development, which can directly induce the degradation of substrate proteins through the in vivo ubiquitin-proteasome protein degradation pathway, and has received widespread attention from researchers.

The mechanism of the PROTAC is that a small molecule inhibitor is connected with a ligand of E3 ubiquitin ligase through a linker to form a combination for targeted induction of protein degradation. In vivo, the inhibitor moiety of this bifunctional molecule can recognize the target protein, and the ligand moiety of E3 can recognize E3 ubiquitin ligase, thereby spatially drawing the target protein close to the E3 ubiquitin ligase, transferring ubiquitin from the E3 ubiquitin ligase to the target protein, ubiquitinating the target protein, and then degrading the target protein via the ubiquitin-proteasome pathway.

In order to expand the application of PROTAC in drug development, the search for a small-molecule ligand of E3 ubiquitin ligase is an important breakthrough. In 2008, Craig et al. reported a PROTAC linking the ligand nutlin of E3 ubiquitin ligase (MDM2) to an androgen receptor inhibitor (SCHNEEKLOTH A R, PUCHEAULT M, TAE H S, et al. Targeted intracellular protein degradation induced by a small molecule: En route to chemical proteomics. Bioorganic & medicinal chemistry letters, 2008, 18(22): 5904-5908). Crews et al. designed and engineered a ligand of E3 ubiquitin ligase (VHL), and found a VHL ligand with high affinity (BUCKLEY D L, GUSTAFSON J L, VAN MOLLE I, et al. Small-molecule inhibitors of the interaction between the E3 ligase VHL and HIF1alpha. Angewandte Chemie, 2012, 51(46): 11463-11467). In 2015, Bradner's research group and Crews' research group reported methods for designing bifunctional molecules dBET1 and ARV-825 using a ligand (domide drug) of E3 ubiquitin ligase (CRBN) and JQ1, an inhibitor of BRD4, respectively (WINTER G E, BUCKLEY D L, PAULK J, et al. DRUG DEVELOPMENT. Phthalimide conjugation as a strategy for in vivo target protein degradation. Science, 2015, 348(6241): 1376-1381; LU J, QIAN Y, ALTIERI M, et al. Hijacking the E3 ubiquitin ligase cereblon to efficiently target brD4. Chemistry & biology, 2015, 22(6): 755-763).

Despite the rapid development of the PROTAC technology, many challenges still remain in the application. For example, ligands of E3 ubiquitin ligases are few.

E3 ubiquitin ligase tripartite motif 25 (TRIM25) is one of the members of the tripartite motif protein family in E3 ubiquitin ligases. At present, reports on the ligand of TRIM25 are rarely found, and no PROTAC using TRIM25 ligand is reported.

### SUMMARY

The inventors of the present invention have discovered a class of perinaphthenone compounds in research, which can bind to E3 ubiquitin ligase tripartite motif 25 (TRIM25) in vitro, promote TRIM25 to recognize PA protein, induce the PA protein to undergo protease-dependent ubiquitination degradation, and have the potential to be used as ligands of E3 ubiquitin ligase TRIM25, realizing wider application, such as in the preparation of PROTAC molecules. Therefore, such compounds have very good application prospects and research and development values. In a first aspect, the present invention provides a compound having the following structure:

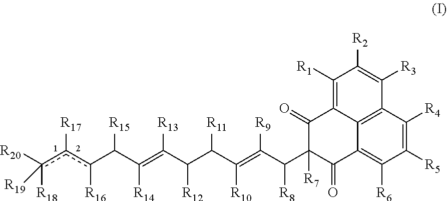

wherein,
the valence bond [symbol] at 1 and 2 represents a single or double bond, and 1 and 2 are not both double bonds;
R₁ to R₁₈ are independently selected from: H, alkyl, alkenyl, cycloalkyl, cycloalkylalkyl, aryl, aralkyl, heterocyclyl, heterocyclylalkyl, halogen, —CN, —NO₂, —COR^{A}, —C(O)OR^{A}, —OCOR^{A}, —C(O)NR^{A}R^{B}, —CH═NR^{A}, —OR^{A}, —OC(O)R^{A}, —S(O)ₜ—R^{A}, —S(O)ₜ—NR^{A}R^{B}, —NR^{A}R^{B}, and —NR^{A}C(O)R^{B}; optionally, the H on each group may be substituted with one or more groups selected from: halogen, —CN, —CF₃, —NO₂, —CHO, —COOH, —C(O)NH₂, —OH, —OC(O)H, —SH, —S(O)₂H, and —NH₂;
R₁₉ and R₂₀ are independently selected from: H, alkyl, alkenyl, cycloalkyl, cycloalkylalkyl, aryl, aralkyl, heterocyclyl, and heterocyclylalkyl; optionally, the H on each group may be substituted with one or more groups selected from: substituted or unsubstituted heterocyclyl, halogen, —CN, —NO₂, —COR^{A}, —C(O)OR^{A}, —C(O)NR^{A}R^{B}, —CH═NR^{A}, —OR^{A}, —OC(O)R^{A}, —S(O)ₜ—R^{A}, —S(O)ₜ—NR^{A}R^{B}, —NR^{A}R^{B}, and —NR^{A}C(O)R^{B}; or, R₁₉ and R₂₀, together with the carbon atom to which they are attached, form substituted or unsubstituted cycloalkyl or heterocyclyl; or, R₁₇ and R₁₉, together with the carbon atoms to which they are attached, form substituted or unsubstituted cycloalkyl or heterocyclyl;
t is selected from 0, 1 and 2;
each R^{A} and R^{B} are independently selected from: H, alkyl, cycloalkyl, alkenyl, aryl, heterocyclyl, and halogen.

Specifically, R₁ to R₁₈ are independently selected from: H, C1-10 alkyl, C1-10 haloalkyl (such as fluoroalkyl, e.g. trifluoromethyl), C1-10 alkenyl, C3-6 cycloalkyl, C4-10 cycloalkylalkyl, phenyl, four- to six-membered heterocycloalkyl, halogen, —CN, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl).

More specifically, R₁ is selected from: —OH, —O(C1-10 alkyl), —SH, —S(C1-10 alkyl), —S(O)₂H, and —S(O)₂(C1-10 alkyl); in some embodiments of the present invention, R₁ is —OH.

More specifically, R₂ is selected from: H, halogen, —CN, —CF₃, —NO₂, —CHO, —COOH, —C(O)NH₂, and —NH₂; in some embodiments of the present invention, R₂ is H.

More specifically, R₃ is selected from: C1-10 alkyl, C1-10 haloalkyl (such as fluoroalkyl, e.g., trifluoromethyl), C1-10 alkenyl, C3-6 cycloalkyl, and C4-10 cycloalkylalkyl, especially C1-10 alkyl, e.g., C1-6 alkyl and C1-3 alkyl (such as methyl, ethyl, n-propyl, and isopropyl); in some embodiments of the present invention, R₃ is methyl.

More specifically, R₄ is selected from: —OH, —O(C1-10 alkyl), —SH, —S(C1-10 alkyl), —S(O)₂H, and —S(O)₂(C1-10 alkyl); in some embodiments of the present invention, R₄ is —OH.

More specifically, R₅ is selected from: H, C1-10 alkyl, C1-10 haloalkyl (such as fluoroalkyl, e.g., trifluoromethyl), C1-10 alkenyl, C3-6 cycloalkyl, and C4-10 cycloalkylalkyl, especially C1-10 alkyl, e.g., C1-6 alkyl and C1-3 alkyl (such as methyl, ethyl, n-propyl, and isopropyl); in some embodiments of the present invention, R₅ is methyl; in other embodiments of the present invention, R₅ is H.

More specifically, R₆ is selected from: —OH, —O(C1-10 alkyl), —SH, —S(C1-10 alkyl), —S(O)₂H, and —S(O)₂(C1-10 alkyl); in some embodiments of the present invention, R₆ is —OH.

More specifically, R₇ is selected from: —OH, —O(C1-10 alkyl), —SH, —S(C1-10 alkyl), —S(O)₂H, and —S(O)₂(C1-10 alkyl); in some embodiments of the present invention, R₇ is —OH.

More specifically, R₈, R₉, R₁₁, R₁₂, R₁₃, R₁₅, and R₁₆ are independently selected from: H, halogen, —CN, —CF₃, —NO₂, —CHO, —COOH, —C(O)NH₂, and —NH₂; in some embodiments of the present invention, R₈, R₉, R₁₁, R₁₂, R₁₃, R₁₅, R₁₆ are all H.

More specifically, R₁₀ and R₁₄ are independently selected from: C1-10 alkyl, C1-10 haloalkyl (such as fluoroalkyl, e.g., trifluoromethyl), C1-10 alkenyl, C3-6 cycloalkyl, and C4-10 cycloalkylalkyl, especially C1-6 alkyl, e.g., C1-3 alkyl (such as methyl, ethyl, n-propyl, and isopropyl); in some embodiments of the present invention, R₁₀ and R₁₄ are both methyl.

More specifically, R₁₇ is selected from: H, —OH, —O(C1-10 alkyl), —SH, —S(C1-10 alkyl), —S(O)₂H, and —S(O)₂(C1-10 alkyl); in some embodiments of the present invention, R₁₇ is H, and in other embodiments of the present invention, R₁₇ is —OH.

More specifically, R₁₈ is selected from: H, C1-10 alkyl, C1-10 haloalkyl (such as fluoroalkyl, e.g., trifluoromethyl), halogen, —CN, —CF₃, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl); optionally, one or more H on each group may be substituted with the following groups selected from: halogen, —CN, —CF₃, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl); more specifically, R₁₈ is selected from: H, C1-6 alkyl, C1-6 haloalkyl (such as fluoroalkyl, e.g., trifluoromethyl), C1-6 hydroxy-substituted alkyl (C1-6 alkyl substituted with one or more hydroxy, e.g., hydroxymethyl), —CHO, —CO(C1-10 alkyl), —COOH, and —C(O)O(C1-10 alkyl); in some embodiments of the present invention, R₁₈ is selected from: H, methyl, ethyl, n-propyl, isopropyl, —CH₂OH, —COOH, —COOCH₃, and —CHO.

In one embodiment of the present invention, R₁₉ has the following structure:

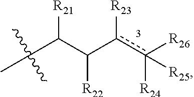

wherein
[symbol] represents a single or double bond;
R₂₁ to R₂₄ are independently selected from: H, C1-10 alkyl, C1-10 haloalkyl (such as fluoroalkyl, e.g. trifluoromethyl), C1-10 alkenyl, C3-6 cycloalkyl, C4-10 cycloalkylalkyl, phenyl, four- to six-membered heterocycloalkyl, halogen, —CN, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl); or, two of R₂₁ to R₂₄, together with the carbon atom(s) therebetween, form substituted or unsubstituted cycloalkyl or heterocyclyl; when [symbol] represents a double bond, R₂₄ is absent;
R₂₅ and R₂₆ are independently selected from: C1-10 alkyl, C1-10 haloalkyl (such as fluoroalkyl, e.g., trifluoromethyl), C1-10 alkenyl, C3-6 cycloalkyl, C4-10 cycloalkylalkyl, phenyl, and four- to six-membered heterocycloalkyl.

Specifically, R₂₅ and R₂₆ are independently selected from C1-10 alkyl, e.g., C1-6 alkyl and C1-3 alkyl; in some embodiments of the present invention, R₂₅ and R₂₆ are both methyl.

In some embodiments of the present invention, R₂₁ is H.

In some embodiments of the present invention, R₁₉ is

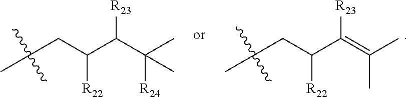

Specifically, R₂₄ is selected from —OH and —O(C1-10 alkyl), or, R₂₃ and R₂₄, together with the carbon atoms therebetween, form substituted or unsubstituted cycloalkyl or heterocyclyl.

Specifically, R₂₂ is selected from: H, —OC(O)H, and —OC(O)(C1-10 alkyl), e.g., —OC(O)CH₃.

Specifically, R₂₃ is selected from: H, —OH, —O(C1-10 alkyl), —SH, and —S(C1-10 alkyl), e.g., H or —OH.

In other embodiments of the present invention, R₂₀ and R₂₂, together with the carbon atoms therebetween, form heterocyclyl.

In other embodiments of the present invention, R₂₀ and R₂₃, together with the carbon atoms therebetween, form heterocyclyl.

Specifically, in the definition of R₁₉ described above, the heterocyclyl is heterocycloalkyl, such as four- to six-membered heterocycloalkyl especially oxygen-containing five- to six-membered heterocycloalkyl, e.g.,

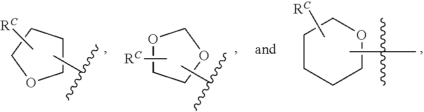

wherein R^{C} is one or more independent substituents on the ring and is selected from: C1-10 alkyl, C1-10 haloalkyl, C1-10 hydroxy-substituted alkyl, C1-10 alkenyl, halogen, —CN, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl).

Specifically, R^{C} is one or more independent substituents on the ring and is selected from: C1-6 alkyl, C1-6 haloalkyl, C1-6 hydroxy-substituted alkyl, C1-6 alkenyl, halogen, —CN, —NO₂, —CHO, —COOH, —C(O)NH₂, —OH, —OC(O)H, and —SH; in some embodiments of the present invention, R^{C} is one or more independent substituents on the ring and is selected from: methyl, ethyl, n-propyl, and isopropyl.

In some embodiments of the resent invention, R₁₉ is selected from:

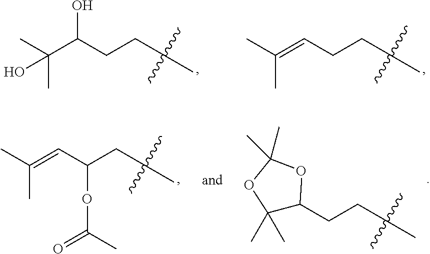

In some embodiments of the present invention, R₂₀ is H.

In some embodiments of the present invention, R₁₉ and R₂₀, together with the carbon atom to which they are attached, form the following group:

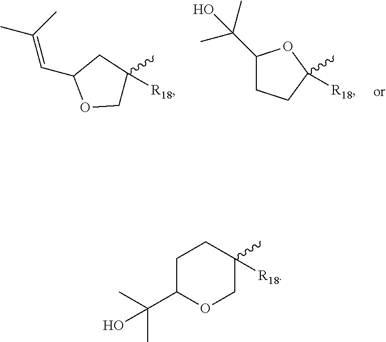

In some embodiments of the present invention, the valence bond at 1 is a double bond and the valence bond at 2 is a single bond.

In other embodiments of the present invention, the valence bond at 1 is a single bond and the valence bond at 2 is a double bond.

In other embodiments of the present invention, the valence bond at 1 is a single bond and the valence bond at 2 is a single bond.

Specifically, the compound may have the following structure:

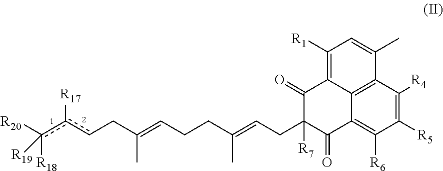

wherein R₁, R₄, R₅, R₆, R₇, R₁₇, R₁₈, R₁₉, and R₂₀ have the corresponding definitions described above in the present invention.

More specifically, the compound may have the following structure:

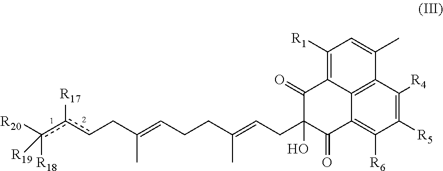

wherein R₁, R₄, R₅, R₆, R₁₇, R₁₈, R₁₉, and R₂₀ have the corresponding definitions described above in the present invention.

Further specifically, the compound may have the following structure:

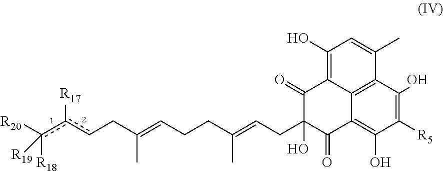

wherein R₅, R₁₇, R₁₈, R₁₉, and R₂₀ have the corresponding definitions described above in the present invention.

More specifically, the compound may be selected from the following structures:

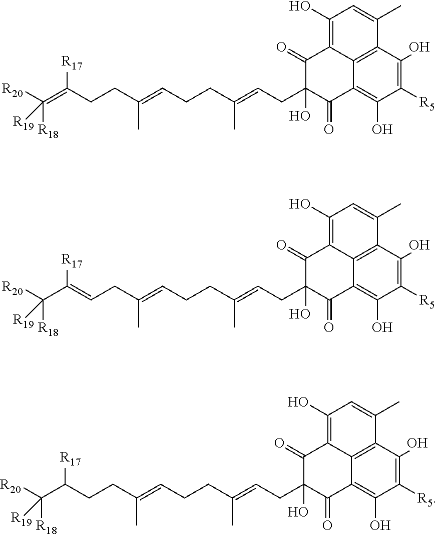

In some embodiments of the present invention, the compound has the following structure:

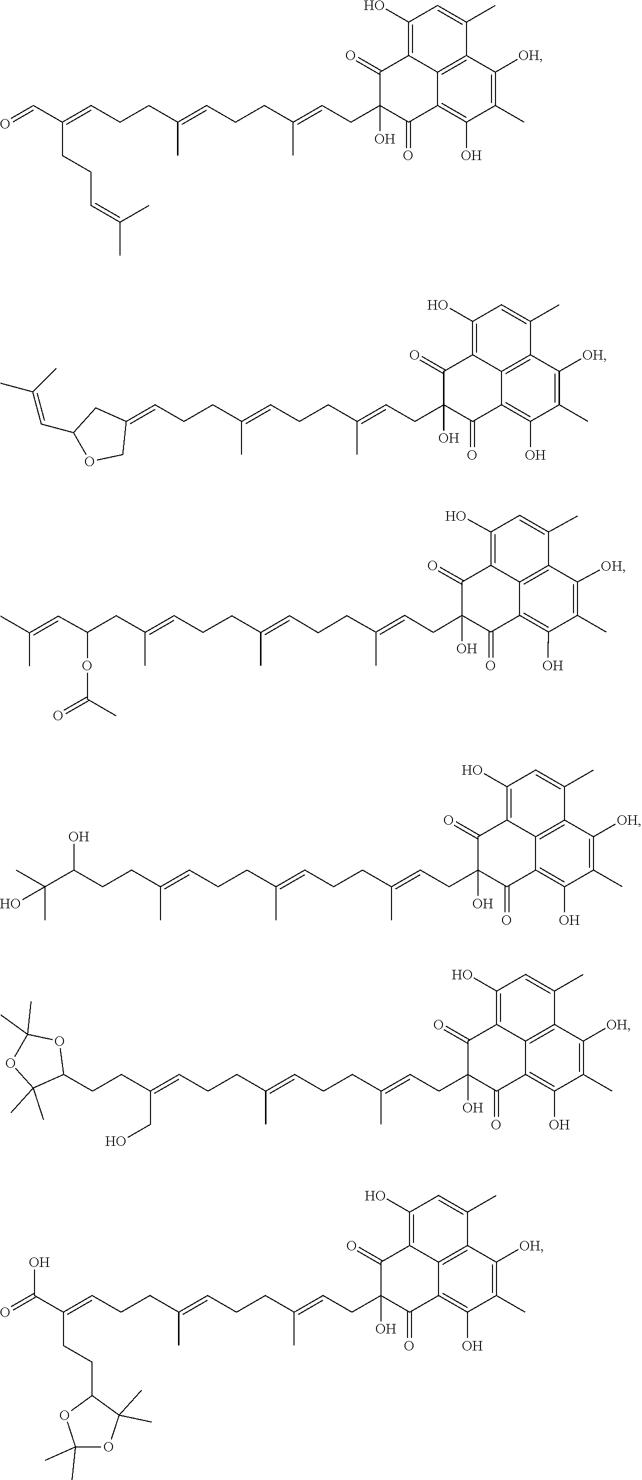

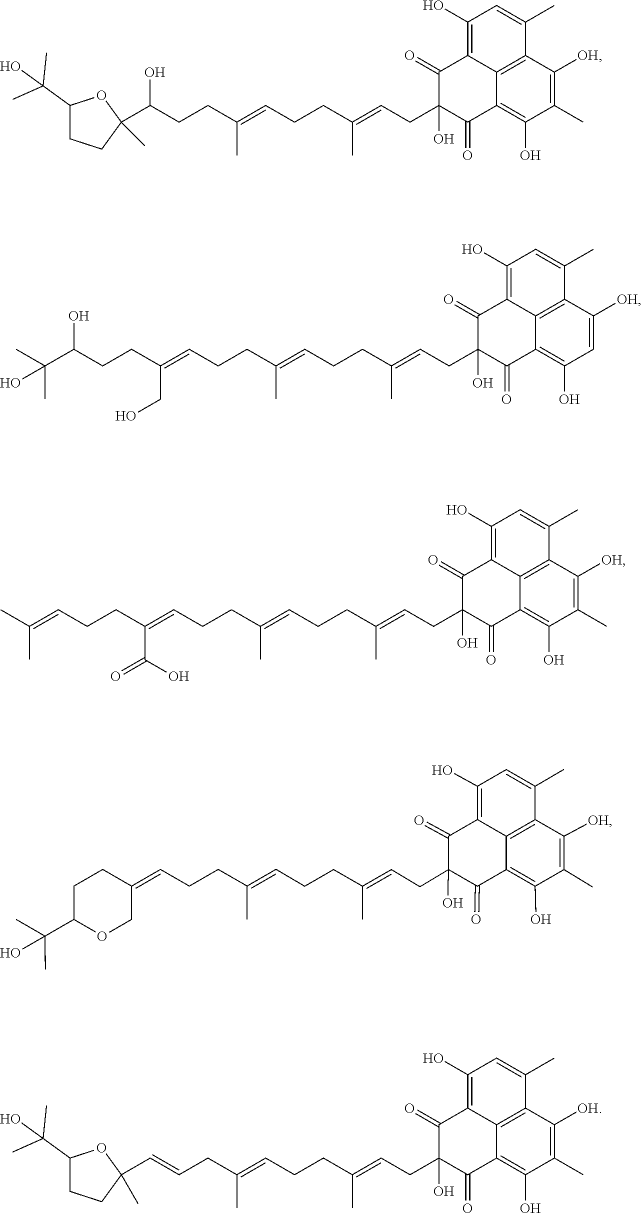

preferably the following compounds are excluded:

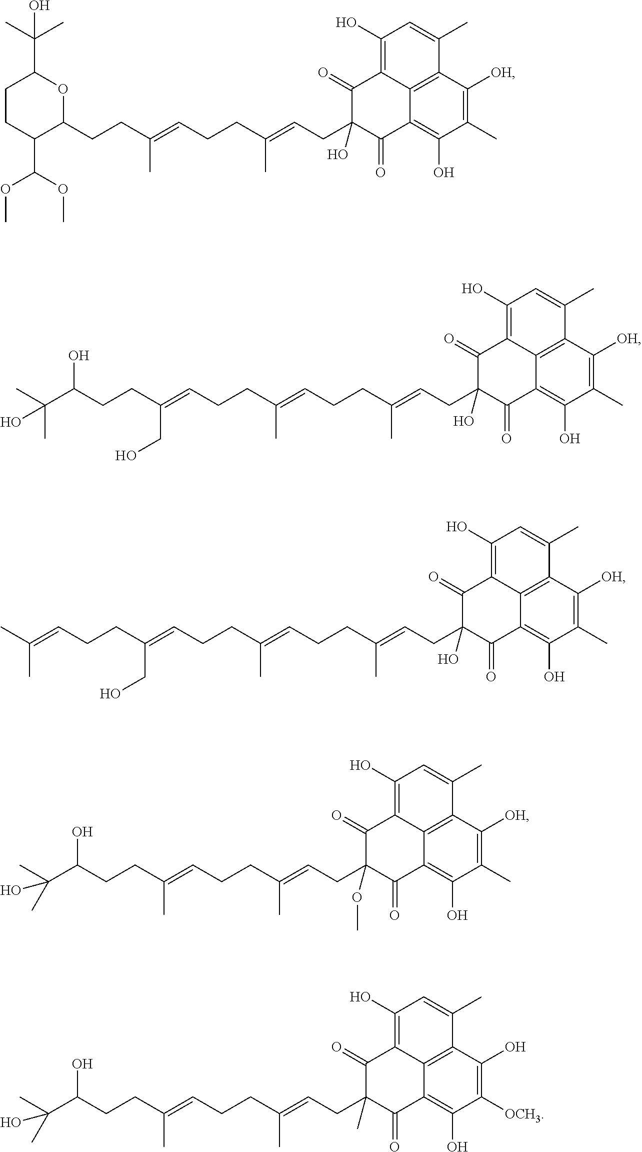

In a second aspect, the present invention provides a pharmaceutically acceptable salt, an ester, a stereoisomer, a prodrug and a solvate of the compound according to the first aspect.

Specifically, the stereoisomer of the compound may have the following structure:

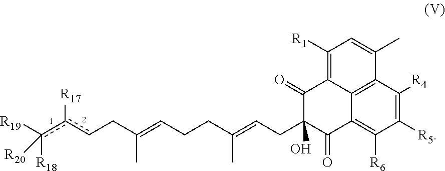

More specifically, the stereoisomer of the compound may have the following structure:

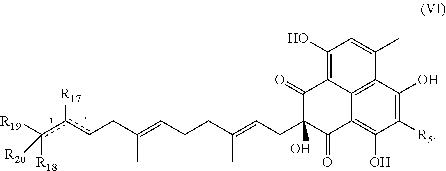

In some embodiments of the present invention, the stereoisomer of the compound has the following structure:

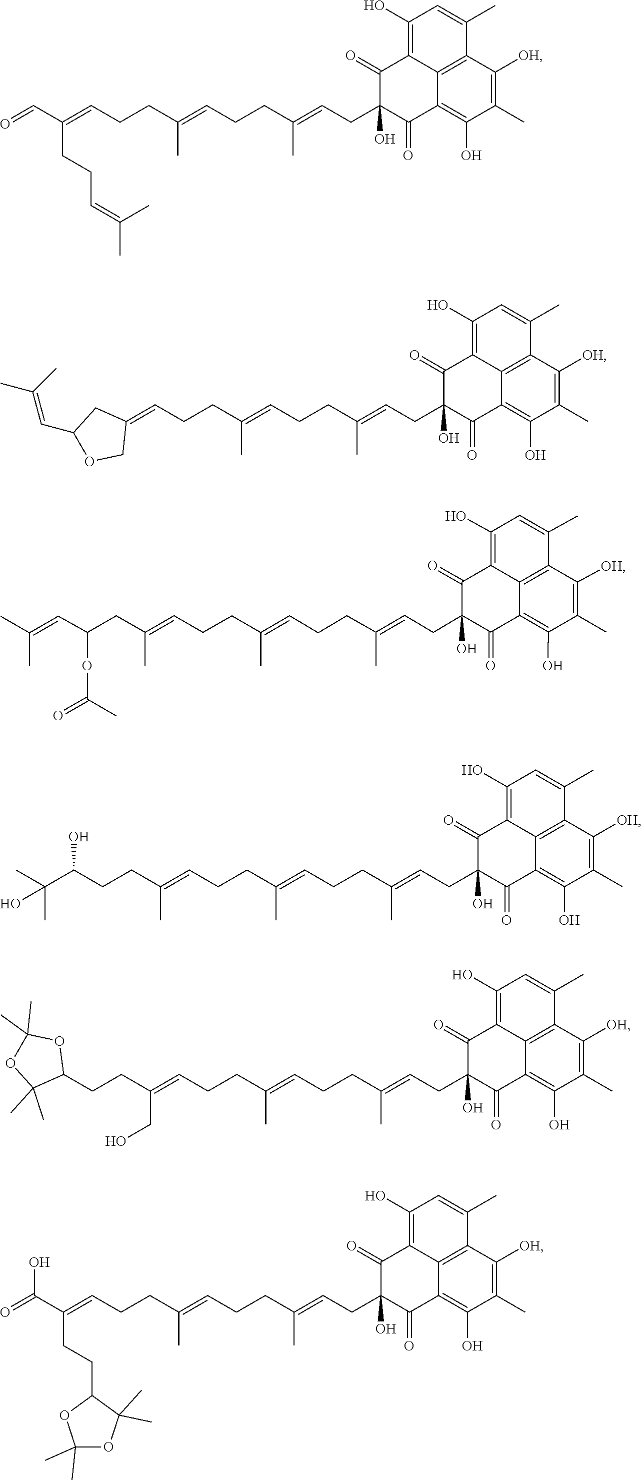

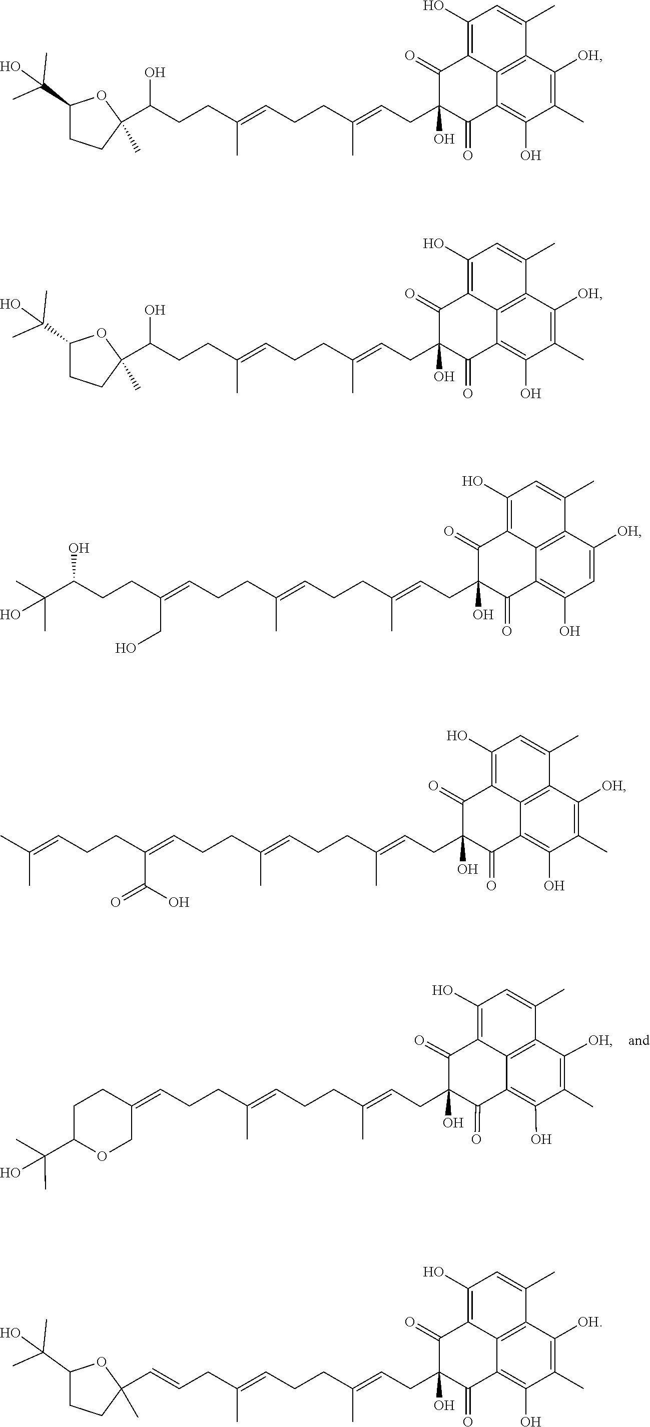

Specifically, the compound according to the first aspect of the present invention and the pharmaceutically acceptable salt, the ester, the stereoisomer, the prodrug and the solvate according to the second aspect of the present invention can be prepared by any suitable method known in the art, such as chemical synthesis, semisynthesis, microbial fermentation or extraction from animals and plants, for example, by extracting and separating the fermentation product of a microorganism (e.g., Aspergillus iizukae CPCC 401321 having an accession number of CGMCC No. 22467), by chemical structure modification (and physical treatment) of the compound obtained by extraction and separation (semisynthesis), or by a series of chemical synthesis and physical treatment processes from chemical materials with relatively simple chemical structures (total synthesis).

In some embodiments of the present invention, the method for preparing the compound according to the first aspect of the present invention may comprise steps of extracting and separating a fermentation product of a microorganism (e.g., Aspergillus iizukae CPCC 401321 having an accession number of CGMCC No. 22467); furthermore, the preparation method may further comprise a step of carrying out chemical structure modification on the compound obtained by extraction and separation.

In other embodiments of the present invention, the method for preparing the compound according to the first aspect of the present invention may comprise a step of preparing the compound by a series of chemical synthesis and physical treatment processes from chemical materials with relatively simple chemical structures (total synthesis).

In a third aspect of the present invention, provided is a pharmaceutical composition comprising the compound according to the first aspect of the present invention or the pharmaceutically acceptable salt, the ester, the stereoisomer, the prodrug and the solvate thereof according to the second aspect of the present invention, and one or more pharmaceutically acceptable excipients.

Specifically, the pharmaceutical composition may be in the form of any suitable formulation, such as, but not limited to, a tablet, a dragee tablet, a film-coated tablet, an enteric-coated tablet, a capsule, a hard capsule, a soft capsule, an oral liquid, a lozenge, a granule, an electuary, a pill, a pulvis, an ointment, a pellet, a suspension, a powder, a solution, an injection, a suppository, an ointment, a plaster, a cream, a spray, a drop, and a patch; oral dosage forms are preferred, such as a capsule, a tablet, an oral liquid, a granule, a pill, a powder, a pellet, and an ointment.

Specifically, the pharmaceutically acceptable excipient may be, such as, but not limited to, a binder, a filler, a diluent, a tabletting agent, a lubricant, a disintegrant, a coloring agent, a flavoring agent, and a wetting agent; suitable fillers may be, for example, cellulose, mannitol, lactose, and other similar fillers; suitable disintegrants may be, for example, starch, polyvinylpyrrolidone, and starch derivatives, which may be, for example, sodium starch glycolate; suitable lubricants may be, for example, magnesium stearate; suitable wetting agents may be, for example, sodium dodecyl sulfate.

Specifically, the pharmaceutical composition may further comprise one or more other ingredients selected from: an inosine monophosphate dehydrogenase (IMPDH) inhibitor, an interferon inducer, an M2 ion channel protein inhibitor, and a neuraminidase inhibitor.

Specifically, the inosine monophosphate dehydrogenase inhibitor may be, for example, ribavirin.

Specifically, the interferon inducer may be, for example, arbidol hydrochloride.

Specifically, the M2 ion channel protein inhibitor may be, for example, amantadine hydrochloride or rimantadine hydrochloride.

Specifically, the neuraminidase inhibitor may be, for example, osehamivir phosphate, Oseltamivir, zanamivir, or peramivir.

In a fourth aspect of the present invention, provided is use of the compound according to the first aspect of the present invention or the pharmaceutically acceptable salt, the ester, the stereoisomer, the prodrug and the solvate thereof according to the second aspect of the present invention, and the pharmaceutical composition according to the third aspect of the present invention in preparing a medicament for preventing and/or treating a disease.

In one embodiment of the present invention, the disease described above is a disease caused by infection of a pathogen.

Specifically, the pathogen described above may be a virus, for example, but not limited to, Adenoviridae (e.g., adenovirus), Herpesviridae (e.g., HSV1 (herpes of mouth), HSV2 (herpes of external genitalia), VZV (chicken pox), EBV (Epstein-Barr virus), CMV (cytomegalovirus)), Poxviridae (e.g., smallpox virus, vaccinia virus), Papovavirus (e.g., human papilloma virus), Parvoviridae (e.g., B19 virus), Hepadnaviridae (e.g., hepatitis B virus), Polyomaviridae (e.g., polyomavirus), Reoviridae (e.g., reovirus, rotavirus), Picornaviridae (e.g., enterovirus, foot-and-mouth disease virus), Caliciviridae (e.g., Norwalk virus, hepatitis E virus), Togaviridae (e.g., rubella virus), Arenaviridae (e.g., lymphocytic choriomeningitis virus), Retroviridae (HIV-1, HIV-2, HTLV-1), Flaviviridae (e.g., Dengue virus, Zika virus, encephalitis B virus, Chikungunya virus, yellow fever virus, hepatitis C virus, West Nile virus), Orthomyxoviridae (e.g., influenza viruses (e.g., influenza A virus, influenza B virus, influenza C virus)), Paramyxoviridae (e.g., human parainfluenza virus type 1 (HPV), HPV type 2, HPV type 3, HPV type 4, Sendai virus, mumps virus, measles virus, respiratory syncytial virus, Newcastle disease virus, etc.), Bunyaviridae (e.g., California encephalitis virus, Hantavirus), Rhabdoviridae (e.g., rabies virus), Filoviridae (e.g., Ebola virus, Marburg virus), Coronaviridae (e.g., HCoV-229E, HCoV-OC43, HCoV-NL63, HCoV-HKU1, SARS-CoV, MERS-CoV, SARS-CoV-2), Astroviridae (e.g., astrovirus), and Bornaviridae (e.g., Borna virus).

In some embodiments of the present invention, the virus is an influenza virus, such as one or more of influenza A virus, influenza B virus, and influenza C virus, in particular influenza A virus.

Specifically, the influenza A virus may be an influenza A virus of H1N1 subtype, H2N2 subtype, H3N2 subtype, H5NI subtype, H7N9 subtype, or H9N2 subtype.

Specifically, the above diseases caused by infection of a virus include, but are not limited to, influenza, SARS, COVID-19, viral hepatitis (such as hepatitis A, hepatitis B, hepatitis C, hepatitis D, etc.), AIDS, rabies, Dengue fever, Ebola virus disease, etc.

In another embodiment of the present invention, the disease described above is a tumor.

Specifically, the above tumor is a malignant tumor, including but not limited to: breast cancer, prostate cancer, ovarian cancer, cervical cancer, skin cancer, pancreatic cancer, colorectal cancer, kidney cancer, liver cancer, brain cancer, lymphoma, leukemia, lung cancer (small cell lung cancer, non-small cell lung cancer), melanoma, stomach cancer, gastroesophageal adenocarcinoma, esophageal cancer, small intestine cancer, cardiac cancer, bladder cancer, anal cancer, gallbladder cancer, bile duct cancer, teratoma, heart tumor, and the like; especially lung cancer (such as non-small cell lung cancer), prostate cancer, liver cancer, breast cancer, stomach cancer, and colorectal cancer.

Specifically, the subject of the medicament may be a mammal (such as a human, a simian, a monkey, a pig, a cow, or a sheep) or a bird (poultry such as a chicken, a duck, or a goose, or a wild bird).

In a fifth aspect of the present invention, provided is use of the compound as shown below and a pharmaceutically acceptable salt, an ester, a stereoisomer, a prodrug and a solvate thereof as a ligand of E3 ubiquitin ligase TRIM25, in regulation of the ubiquitination level of a target, and in preparation of a proteolysis targeting chimera (PROTAC),

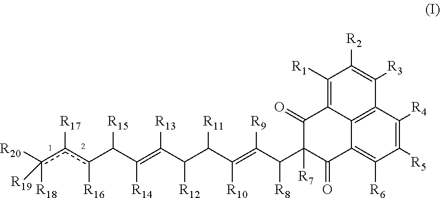

wherein,
the valence bond [symbol] at 1 and 2 represents a single or double bond, and 1 and 2 are not both double bonds;
R₁ to R₁₈ are independently selected from: H, alkyl, alkenyl, cycloalkyl, cycloalkylalkyl, aryl, aralkyl, heterocyclyl, heterocyclylalkyl, halogen, —CN, —NO₂, —COR^{A}, —C(O)OR^{A}, —OCOR^{A}, —C(O)NR^{A}R^{B}, —CH═NR^{A}, —OR^{A}, —OC(O)R^{A}, —S(O)ₜ—R^{A}, —S(O)ₜ—NR^{A}R^{B}, —NR^{A}R^{B}, and —NR^{A}C(O)R^{B}; optionally, the H on each group may be substituted with one or more groups selected from: halogen, —CN, —CF₃, —NO₂, —CHO, —COOH, —C(O)NH₂, —OH, —OC(O)H, —SH, —S(O)₂H, and —NH₂;
R₁₉ and R₂₀ are independently selected from: H, alkyl, alkenyl, cycloalkyl, cycloalkylalkyl, aryl, aralkyl, heterocyclyl, and heterocyclylalkyl; optionally, the H on each group may be substituted with one or more groups selected from: substituted or unsubstituted heterocyclyl, halogen, —CN, —NO₂, —COR^{A}, —C(O)OR^{A}, —C(O)NR^{A}R^{B}, —CH═NR^{A}, —OR^{A}, —OC(O)R^{A}, —S(O)ₜ—R^{A}, —S(O)ₜ—NR^{A}R^{B}, —NR^{A}R^{B}, and —NR^{A}C(O)R^{B}; or, R₁₉ and R₂₀, together with the carbon atom to which they are attached, form substituted or unsubstituted cycloalkyl or heterocyclyl; or, R₁₇ and R₁₉, together with the carbon atoms to which they are attached, form substituted or unsubstituted cycloalkyl or heterocyclyl;
t is selected from 0, 1 and 2;
each R^{A} and R^{B} are independently selected from: H, alkyl, cycloalkyl, alkenyl, aryl, heterocyclyl, and halogen.

Specifically, each group has the corresponding definition as described in the first aspect of the present invention.

Specifically, the target is a target protein to be degraded, which may be a self protein in the body, or a foreign protein, such as a viral protein.

In one embodiment of the present invention, the regulation of the ubiquitination level comprises promoting ubiquitination of PA protein, promoting binding of PA protein to E3 ubiquitin ligase TRIM25, acting as a ligand of E3 ubiquitin ligase TRIM25, and the like.

In some embodiment of the present invention, in the above uses, the compound has the following structure:

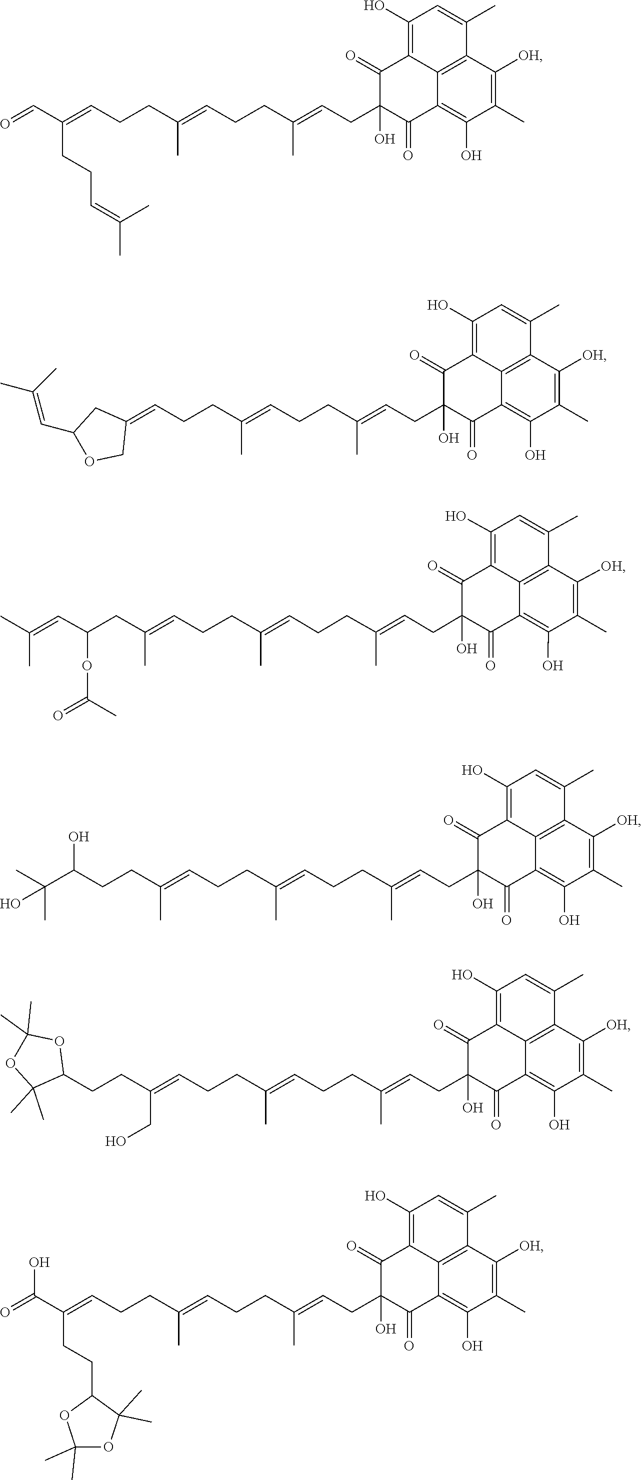

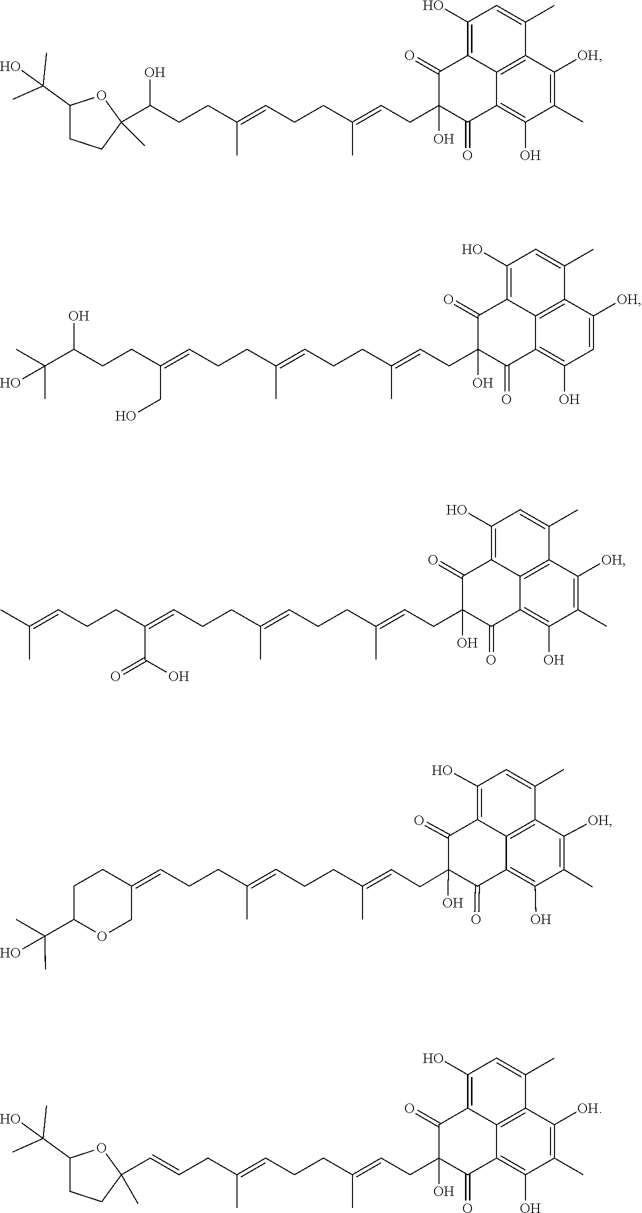

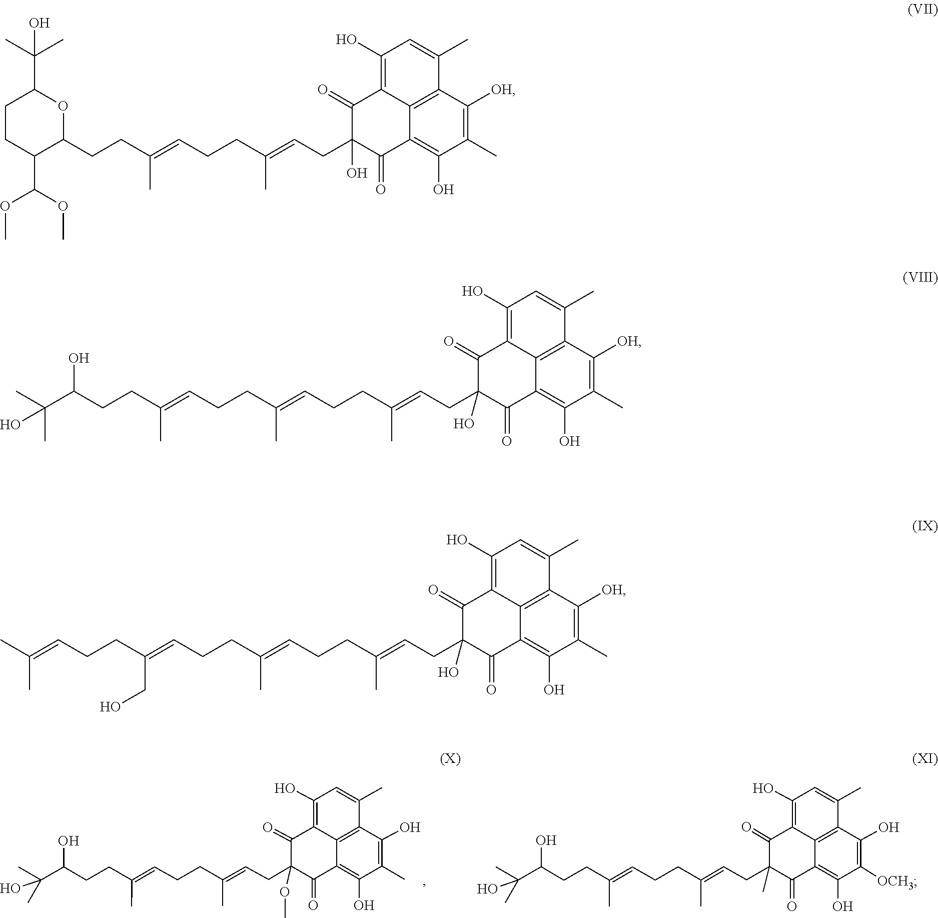 [SMILES: COC(OC)C1CCC(C(C)(C)O)OC1CC/C(C)=C/CC/C(C)=C/CC1(O)C(=O)c2c(O)cc(C)c3c(O)c(C)c(O)c(c23)C1=O ; C/C(=C\CC/C(C)=C/CC1(O)C(=O)c2c(O)cc(C)c3c(O)c(C)c(O)c(c23)C1=O)CC/C=C(\C)CCC(O)C(C)(C)O ; CC(C)=CCC/C(=C/CC/C(C)=C/CC/C(C)=C/CC1(O)C(=O)c2c(O)cc(C)c3c(O)c(C)c(O)c(c23)C1=O)CO ; COC1(C/C=C(\C)CC/C=C(\C)CCC(O)C(C)(C)O)C(=O)c2c(O)cc(C)c3c(O)c(C)c(O)c(c23)C1=O ; COc1c(O)c2c3c(c(O)cc(C)c3c1O)C(=O)C(C)(C/C=C(\C)CC/C=C(\C)CCC(O)C(C)(C)O)C2=O]

in particular

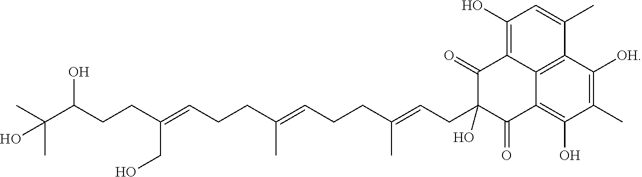 [SMILES: C/C(=C\CC/C(C)=C/CC1(O)C(=O)c2c(O)cc(C)c3c(O)c(C)c(O)c(c23)C1=O)CC/C=C(\CO)CCC(O)C(C)(C)O]

In some embodiments of the present invention, in the above uses, the stereoisomer of the compound has the following structure:

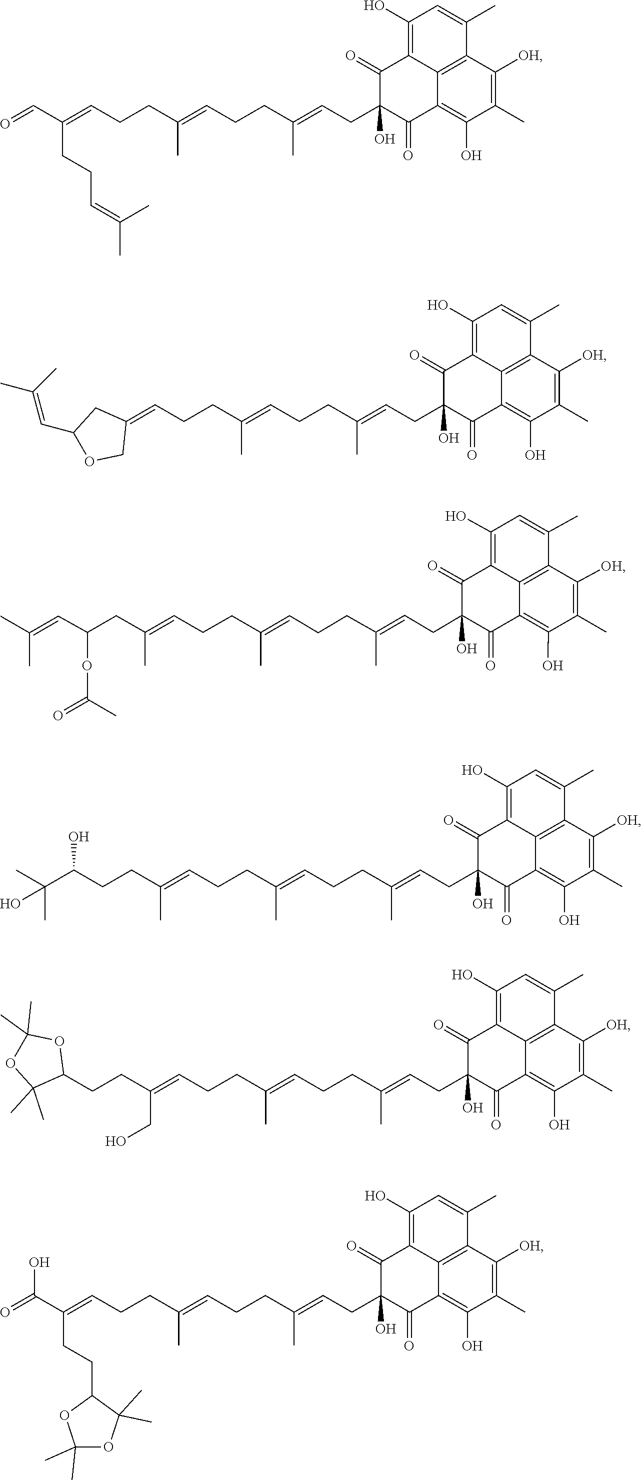

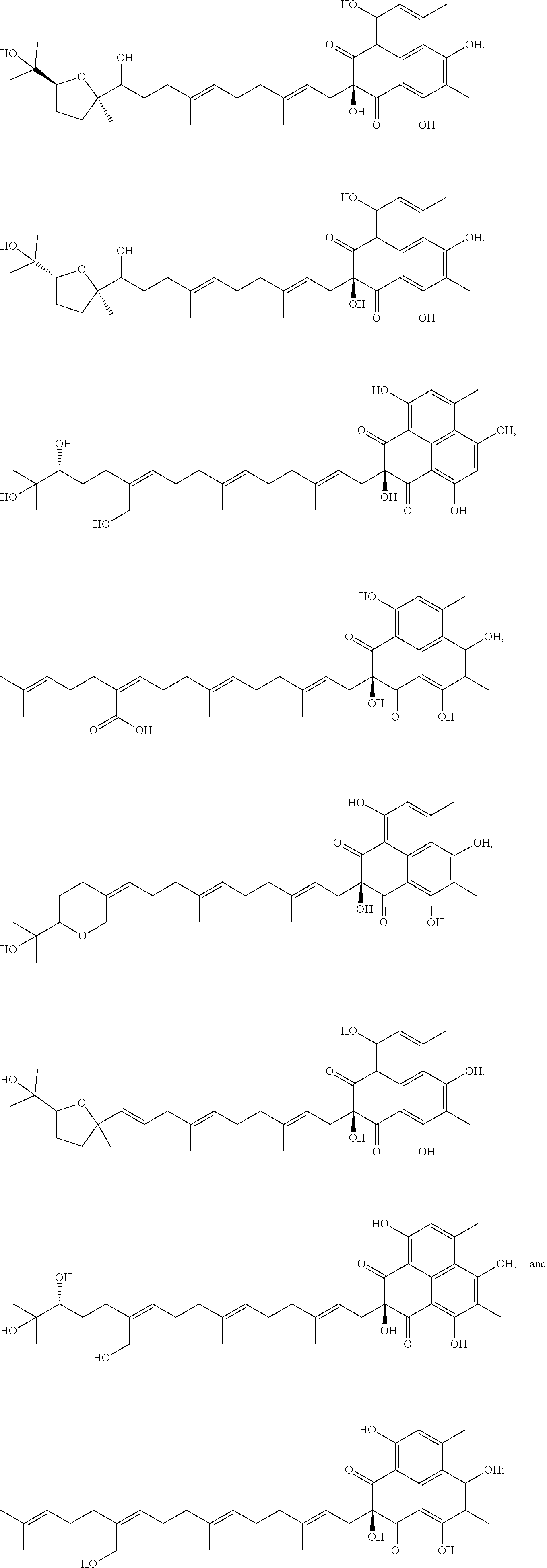

and
in particular

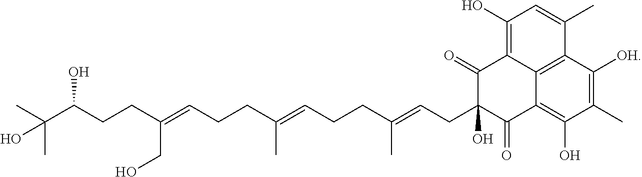 [SMILES: C/C(=C\CC/C(C)=C/C[C@]1(O)C(=O)c2c(O)cc(C)c3c(O)c(C)c(O)c(c23)C1=O)CC/C=C(\CO)CC[C@@H](O)C(C)(C)O]

Specifically, in the above uses, the PROTAC may have the following structure: SMI-L-E₃L, wherein SMI is a small molecule inhibitor moiety (which may be formed from any suitable small molecule inhibitor of the target known in the art), E₃L is a ligand moiety of E3 ubiquitin ligase (e.g., a structural moiety formed from the compound or the pharmaceutically acceptable salt, the stereoisomer, the ester, the prodrug, and the solvate thereof described above), and L is a linking bond or linking group between SMI and E₃L.

In a sixth aspect of the present invention, provided is a PROTAC having the following structure:

SMI-L-E₃L (XII)

wherein SMI is a small molecule inhibitor moiety;
E₃L is a ligand moiety of E3 ubiquitin ligase, which is formed from the compound or the pharmaceutically acceptable salt, the stereoisomer, the ester, the prodrug, and the solvate thereof described in the sixth aspect of the present invention;
L is a linking bond or linking group between SMI and E₃L.

Specifically, SMI may be formed from any suitable small molecule inhibitor of the target known in the art.

In a seventh aspect of the present invention, provided is a method for preparing PROTAC, comprising a step of using the compound or the pharmaceutically acceptable salt, the stereoisomer, the ester, the prodrug, and the solvate thereof according to the sixth aspect of the present invention.

In an eighth aspect of the present invention, provided is an aspergillus, which has been deposited at the China General Microbiological Culture Collection Center (address: No. 3, Yard No. 1, West Beichen Road, Chaoyang District, Beijing, Institute of Microbiology, Chinese Academy of Sciences) on Jul. 8, 2021, with an accession number of CGMCC No. 22467, and classification and naming of Aspergillus iizukae.

In a ninth aspect of the present invention, provided is a method for preventing and/or treating a disease, comprising a step of administering to a subject in need thereof an effective amount of the compound or the pharmaceutically acceptable salt, the stereoisomer, the ester, the prodrug, and the solvate according to the first aspect of the present invention, the pharmaceutical composition according to the third aspect of the present invention, or the PROTAC according to the sixth aspect of the present invention.

Specifically, the above disease may be a disease caused by infection of a pathogen, such as influenza, SARS, COVID-19, viral hepatitis (such as hepatitis A, hepatitis B, hepatitis C, hepatitis D, etc.), AIDS, rabies, Dengue fever, Ebola virus disease, etc.; a tumor, such as breast cancer, prostate cancer, ovarian cancer, cervical cancer, skin cancer, pancreatic cancer, colorectal cancer, kidney cancer, liver cancer, brain cancer, lymphoma, leukemia, lung cancer (small cell lung cancer, non-small cell lung cancer), melanoma, stomach cancer, gastroesophageal adenocarcinoma, esophageal cancer, small intestine cancer, cardiac cancer, bladder cancer, anal cancer, gallbladder cancer, bile duct cancer, teratoma, heart tumor, and the like.

Specifically, the subject described above may be a mammal (such as a human, a simian, a monkey, a pig, a cow, or a sheep) or a bird (poultry such as a chicken, a duck, or a goose, or a wild bird).

In a tenth aspect of the present invention, provided is a method for regulating the ubiquitination level of a target, comprising a step of administering to a subject in need thereof an effective amount of the compound or the pharmaceutically acceptable salt, the stereoisomer, the ester, the prodrug, and the solvate according to the first aspect of the present invention, the pharmaceutical composition according to the third aspect of the present invention, or the PROTAC according to the sixth aspect of the present invention.

The present invention provides a class of perinaphthenone compounds, which can bind to TRIM25, promote TRIM25 to recognize pathogen proteins (such as viruses), induce the pathogen to undergo protease-dependent ubiquitination degradation, and are expected to be used as ligands of E3 ubiquitin ligase TRIM25 to realize wider application, such as preparation of PROTAC molecules. Therefore, the compounds have very good application prospects and research and development values.

The deposit information of the biomaterial in the present invention is as follows:

An aspergillus, Aspergillus iizukae CPCC 401321, which has been deposited at the China General Microbiological Culture Collection Center (address: No. 3, Yard No. 1, West Beichen Road, Chaoyang District, Beijing, Institute of Microbiology, Chinese Academy of Sciences) on Jul. 8, 2021, with an accession number of CGMCC No. 22467, and classification and naming of Aspergillus iizukae.

### BRIEF DESCRIPTION OF THE DRAWINGS

FIG. 1 shows the ¹H-NMR spectrum of compound C1.

FIG. 2 shows the ¹³C-NMR spectrum of compound C1.

FIG. 3 shows the ¹H-NMR spectrum of compound C2.

FIG. 4 shows the ¹³C-NMR spectrum of compound C2.

FIG. 5 shows the ¹H-NMR spectrum of compound C3.

FIG. 6 shows the ¹³C-NMR spectrum of compound C3.

FIG. 7 shows the HSQC spectrum of compound C3.

FIG. 8 shows the HMBC spectrum of compound C3.

FIG. 9 shows the ¹H-¹H COSY spectrum of compound C3.

FIG. 10 shows the NOESY spectrum of compound C3.

FIG. 11 shows the ¹H-NMR spectrum of compound C4.

FIG. 12 shows the ¹³C-NMR spectrum of compound C4.

FIG. 13 shows the HSQC spectrum of compound C4.

FIG. 14 shows the HMBC spectrum of compound C4.

FIG. 15 shows the ¹H-¹H COSY spectrum of compound C4.

FIG. 16 shows the NOESY spectrum of compound C4.

FIG. 17 shows the ¹H-NMR spectrum of compound C5.

FIG. 18 shows the ¹³C-NMR spectrum of compound C5.

FIG. 19 shows the HSQC spectrum of compound C5.

FIG. 20 shows the HMBC spectrum of compound C5.

FIG. 21 shows the ¹H-¹H COSY spectrum of compound C5.

FIG. 22 shows the NOESY spectrum of compound C5.

FIG. 23 shows the ¹H-NMR spectrum of compound C6.

FIG. 24 shows the ¹³C-NMR spectrum of compound C6.

FIG. 25 shows the HSQC spectrum of compound C6.

FIG. 26 shows the HMBC spectrum of compound C6.

FIG. 27 shows the ¹H-¹H COSY spectrum of compound C6.

FIG. 28 shows the NOESY spectrum of compound C6.

FIG. 29 shows the ¹H-NMR spectrum of compound C7.

FIG. 30 shows the ¹³C-NMR spectrum of compound C7.

FIG. 31 shows the HSQC spectrum of compound C7.

FIG. 32 shows the HMBC spectrum of compound C7.

FIG. 33 shows the ¹H-¹H COSY spectrum of compound C7.

FIG. 34 shows the NOESY spectrum of compound C7.

FIG. 35 shows the ¹H-NMR spectrum of compound C8.

FIG. 36 shows the ¹³C-NMR spectrum of compound C8.

FIG. 37 shows the HSQC spectrum of compound C8.

FIG. 38 shows the HMBC spectrum of compound C8.

FIG. 39 shows the ¹H-¹H COSY spectrum of compound C8.

FIG. 40 shows the NOESY spectrum of compound C8.

FIG. 41 shows the ¹H-NMR spectrum of compound C9.

FIG. 42 shows the ¹³C-NMR spectrum of compound C9.

FIG. 43 shows the HSQC spectrum of compound C9.

FIG. 44 shows the HMBC spectrum of compound C9.

FIG. 45 shows the ¹H-¹H COSY spectrum of compound C9.

FIG. 46 shows the NOESY spectrum of compound C9.

FIG. 47 shows the ¹H-NMR spectrum of compound C10.

FIG. 48 shows the ¹³C-NMR spectrum of compound C10.

FIG. 49 shows the HSQC spectrum of compound C10.

FIG. 50 shows the HMBC spectrum of compound C10.

FIG. 51 shows the ¹H-¹H COSY spectrum of compound C10.

FIG. 52 shows the ¹H-NMR spectrum of compound C11.

FIG. 53 shows the ¹³C-NMR spectrum of compound C11.

FIG. 54 shows the HSQC spectrum of compound C11.

FIG. 55 shows the HMBC spectrum of compound C11.

FIG. 56 shows the ¹H-¹H COSY spectrum of compound C11.

FIG. 57 shows the ¹H-NMR spectrum of compound C12.

FIG. 58 shows the ¹³C-NMR spectrum of compound C12.

FIG. 59 shows the HSQC spectrum of compound C12.

FIG. 60 shows the HMBC spectrum of compound C12.

FIG. 61 shows the ¹H-¹H COSY spectrum of compound C12.

FIG. 62 shows the NOESY spectrum of compound C12.

FIG. 63 shows the ¹H-NMR spectrum of compound C13.

FIG. 64 shows the ¹³C-NMR spectrum of compound C13.

FIG. 65 shows the HSQC spectrum of compound C13.

FIG. 66 shows the HMBC spectrum of compound C13.

FIG. 67 shows the ¹H-¹H COSY spectrum of compound C13.

FIG. 68 shows the NOESY spectrum of compound C13.

FIG. 69 shows the ¹H-NMR spectrum of compound C14.

FIG. 70 shows the ¹³C-NMR spectrum of compound C14.

FIG. 71 shows the HSQC spectrum of compound C14.

FIG. 72 shows the HMBC spectrum of compound C14.

FIG. 73 shows the ¹H-¹H COSY spectrum of compound C14.

FIG. 74 shows the NOESY spectrum of compound C14.

FIG. 75 shows the experimental results showing the effect of compounds C1-C14 on the expression of influenza virus PA protein.

FIG. 76 shows the experimental results showing that compound C1 down-regulates the PA protein degradation pathway.

FIG. 77 shows the experimental results showing that compound C1 induces polyubiquitination of PA.

FIG. 78 shows the experimental results showing that compound C1 induces degradation of PA by recognizing the E3 ligase TRIM25.

FIG. 79 shows the experimental results showing that compound C1 promotes the interaction of TRIM25 with PA.

FIG. 80 shows the experimental results showing that the compounds bind to TRIM25 in vitro.

FIG. 81 shows the experimental results showing the ability of the compounds to bind to PA in vitro.

FIG. 82 shows the experimental results showing that compound C1 promotes the polyubiquitination level of PA protein in vitro.

### DETAILED DESCRIPTION

Unless otherwise defined, all scientific and technical terms used in the present invention have the same meaning as commonly understood by those skilled in the art to which the present invention relates.

The term “alkyl” refers to a hydrocarbon group that is linear or branched and that does not contain unsaturated bonds, and that is linked to the rest of the molecule via a single bond. The alkyl as used herein typically contains 1 to 10 (e.g., 1, 2, 3, 4, 5, 6, 7, 8, 9, or 10) carbon atoms (i.e., C₁₋₁₀ alkyl), preferably 1 to 6 carbon atoms (i.e., C₁₋₆ alkyl). Examples of alkyl include, but are not limited to, methyl, ethyl, n-propyl, isopropyl, n-butyl, isobutyl, tert-butyl, n-pentyl, isopentyl, neopentyl, tert-pentyl, n-hexyl, isohexyl, and the like. If alkyl is substituted with cycloalkyl, it is correspondingly “cycloalkylalkyl”, such as cyclopropylmethyl, cyclopropylethyl, cyclobutylmethyl, cyclopentylmethyl, or cyclohexylmethyl. If alkyl is substituted with aryl, it is correspondingly “aralkyl”, such as benzyl, benzhydryl or phenethyl. If alkyl is substituted with heterocyclyl, it is correspondingly “heterocyclylalkyl”.

The term “alkenyl” refers to a hydrocarbon group that is linear or branched and contains at least two carbon atoms and at least one unsaturated bond, and that is linked to the rest of the molecule via a single bond. The alkenyl as used herein typically contains 1 to 10 (e.g., 1, 2, 3, 4, 5, 6, 7, 8, 9, or 10) carbon atoms (i.e., C₁₋₁₀ alkenyl), preferably 1 to 6 carbon atoms (i.e., C₁₋₆ alkenyl). Examples of alkenyl include, but are not limited to, ethenyl, 1-methyl-ethenyl, 1-propenyl, 2-propenyl, or butenyl, and the like.

The term “cycloalkyl” refers to an alicyclic hydrocarbon, and the cycloalkyl as used herein typically contains 1 to 4 monocyclic and/or fused rings, and 3 to 18 carbon atoms, preferably 3 to 10 (e.g., 3, 4, 5, 6, 7, 8, 9, or 10) carbon atoms (e.g., C₃₋₁₀ cycloalkyl, C₃₋₆ cycloalkyl), such as cyclopropyl, cyclobutyl, cyclopentyl, cyclohexyl, cycloheptyl, cyclooctyl, or adamantyl.

The term “aryl” refers to any functional group or substituent derived from a simple aromatic ring, including monocyclic aryl groups and/or fused aryl groups, such as those containing 1 to 3 monocyclic or fused rings, and having 6 to 18 (e.g., 6, 8, 10, 12, 14, 16, or 18) carbon ring atoms. The aryl as used herein is typically an aryl group that contains 1 to 2 monocyclic or fused rings and has 6 to 12 carbon ring atoms (i.e., C₆₋₁₂ aryl), wherein H on the carbon atoms may be substituted, for example, with alkyl, halogen, and other groups. Examples of aryl include, but are not limited to, phenyl, p-methylphenyl, naphthyl, biphenyl, indenyl, and the like.

The term “halogen” refers to bromine, chlorine, iodine, or fluorine.

The term “heterocyclyl” refers to a 3- to 18-membered non-aromatic ring group containing 2 to 17 carbon atoms and 1 to 10 heteroatoms. Heterocyclyl may be a monocyclic, bicyclic, tricyclic or tetracyclic ring system, which may include fused, spiro or bridged ring systems. Heterocyclyl may be partially saturated (heteroaryl) or fully saturated (heterocycloalkyl). Suitable heteroaryl groups for the compound of the present invention contain 1, 2 or 3 heteroatoms selected from N, O and S atoms and include, for example, coumarin, including 8-coumarin, quinolyl, including 8-quinolyl, isoquinolyl, pyridinyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl, and furopyridinyl. Suitable heterocycloalkyl groups for the compound of the present invention contain 1, 2 or 3 heteroatoms selected from N, O and S atoms and include, for example, pyrrolidinyl, tetrahydrofuryl, dihydrofuran, tetrahydrothienyl, tetrahydrothiopyranyl, piperidinyl, morpholinyl, thiomorpholinyl, oxathianyl, piperazinyl, azetidinyl, oxetanyl, thietanyl, homopiperidinyl, oxiranyl, thiiranyl, azepinyl, oxazepanyl, diazepinyl, triazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

The pharmaceutically acceptable salts of the present invention include acid addition salts and base addition salts.

The acid addition salts include, but are not limited to, salts derived from inorganic acids, such as hydrochloric acid, nitric acid, phosphoric acid, sulfuric acid, hydrobromic acid, hydroiodic acid, and phosphonic acid, and salts derived from organic acids, such as aliphatic mono-carboxylic acid and aliphatic dicarboxylic acid, phenyl-substituted alkanoic acid, hydroxyalkanoic acid, alkanedioic acids, aromatic acid, aliphatic sulfonic acid and aromatic sulfonic acid. Thus, these salts include, but are not limited to, sulfate, pyrosulfate, bisulfate, sulfite, bisulfite, nitrate, phosphate, monohydrogenphosphate, dihydrogenphosphate, metaphosphate, pyrophosphate, hydrochloride, hydrobromide, iodate, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, tosylate, phenylacetate, citrate, lactate, maleate, tartrate, and methanesulfonate, and salts comprising amino acids such as arginate, gluconate and galacturonate. Acid addition salts can be prepared by contacting the free base form with a sufficient amount of the desired acid to form the salt in a conventional manner.

The free base form can be regenerated by contacting the salt form with a base and isolating the free base in a conventional manner.

The base addition salts according to the present invention are salts with metals or amines, such as hydroxides of alkali metals and alkaline earth metals, or with organic amines. Examples of metals used as cations include, but are not limited to, sodium, potassium, magnesium and calcium. Examples of suitable amines include, but are not limited to, N,N-dibenzylethylenediamine, chloroprocaine, choline, diethanolamine, ethylenediamine(ethane-1,2-diamine), N-methylglucamine and procaine. Base addition salts can be prepared by contacting the free acid form with a sufficient amount of the desired base to form the salt in a conventional manner. The free acid form can be regenerated by contacting the salt form with an acid and isolating the free acid in a conventional manner.

The stereoisomer described herein includes enantiomeric, diastereomeric and geometric forms. Some of the compounds of the present invention have cyclohydrocarbyl which may be substituted on more than one carbon atom, in which case all geometric forms thereof, including cis and trans, and mixtures thereof, are within the scope of the present invention. The cyclohydrocarbyl includes aliphatic cyclohydrocarbyl and aryl, wherein the alicyclic cyclohydrocarbyl may be non-aromatic, monocyclic, fused, bridged or spiro, saturated or unsaturated cyclic hydrocarbyl, and the aryl may be phenyl, naphthyl, phenanthryl, biphenyl and the like.

The solvate described herein refers to a physical association of the compound of the present invention with one or more solvent molecules. The physical association includes various degrees of ionic and covalent bonding, including hydrogen bonding. In some cases, the solvate can be isolated, for example, when one or more solvent molecules are incorporated into the crystal lattice of a crystalline solid. Solvates include both solution phases and isolatable solvates. Representative solvates include ethanolates, methanolates, and the like.

The prodrug described herein refers to forms of the compound of formula I (including acetals, esters, and zwitterions) which are suitable for administration to patients without undue toxicity, irritation, allergic response and the like, and which are effective for the intended use thereof. The prodrug is converted in vivo, e.g., by hydrolysis in blood, to give the parent compound.

The “patient” or “subject” and the like described herein are used interchangeably herein and refer to any animal or a cell thereof, whether in vitro or in situ, treated according to the method described herein. Specifically, the aforementioned animal includes mammals, for example, rats, mice, guinea pigs, rabbits, dogs, monkeys, or humans, particularly humans.

The “treating” described herein refers to preventing, curing, reversing, attenuating, alleviating, minimizing, suppressing, arresting, and/or stopping one or more clinical symptoms of a disease after its onset.

The “preventing” described herein refers to treatment to avoid, minimize, or make difficult the onset or progression of a disease prior to its onset.

The disclosures of the various publications, patents, and published patent specifications cited herein are hereby incorporated by reference in their entireties.

The technical solutions of the present invention will be clearly and completely described below with reference to the examples of the present invention, and it is obvious that the described examples are only a part of the examples of the present invention but not all of them. Based on the examples of the present invention, all other examples obtained by those of ordinary skills in the art without creative work shall fall within the protection scope of the present invention.

### Example 1: Preparation of Aspergillus iizukae CPCC 401321 Fermentation Culture

Aspergillus iizukae CPCC 401321 is a high-yield strain for perinaphthenone compounds (having an accession number of CGMCC No. 22467). The strain was cultured at 28° C. for 7 days on a PDA slant, then a mycelium block was picked and inoculated into a 500 mL triangular flask containing 100 mL of PDB seed culture medium, and the mixture was cultured with shaking at 28-30° C. for 5 days to serve as a seed liquid. Then 10 mL of the seed liquid was inoculated into a 500 mL triangular flask containing a rice culture medium (the rice culture medium was obtained by adding 100 g of rice into the 500 mL triangular flask, adding 100 mL of deionized water, soaking, and sterilizing at 121° C. for 20 min), and the mixture was cultured at 28° C. for 20 days to obtain a solid fermentation culture of Aspergillus iizukae CPCC 401321.

### Example 2: Preparation of Ethyl Acetate Extract of Aspergillus iizukae CPCC 401321 Fermentation Culture

Ten kg of the Aspergillus iizukae CPCC 401321 solid fermentation culture obtained in Example 1 was taken and crushed with a glass rod, added with 20 L of ethyl acetate, and subjected to ultrasonic extraction at room temperature for 3 times, each time for 30 min. The ethyl acetate extracts were combined, and subjected to rotary evaporation using a rotary evaporator (temperature: 40° C.) to remove ethyl acetate in the extract, The obtained substance was the ethyl acetate extract of Aspergillus iizukae CPCC 401321 fermentation culture.

### Example 3: Separation of Ethyl Acetate Extract

(1) The ethyl acetate extract (250 g) in Example 2 was dissolved in an ethyl acetate-methanol mixed solution, and then separated by silica gel column chromatography. The silica gel used in the silica gel column chromatography was silica gel H, the silica gel column had a specification of 12×40 cm, and a column volume of 4522 mL. The elution procedure used in the silica gel column chromatography was a linear gradient elution as follows: the mobile phase used was a mixed solution composed of petroleum ether and acetone, and the volume ratio of petroleum ether to acetone in the mobile phase of the linear gradient elution was linearly reduced from 4:1 to 1:1. The eluate was collected without interruption from the beginning of the elution procedure according to 500 mL each fraction (200 mL/fraction), and 275 fractions were continuously collected, recorded as: Fr.1, Fr.2, Fr.3, Fr.4, . . . , and Fr.275, respectively. The same fractions were combined under the guidance of TLC detection, resulting in 19 combined fractions: Fr.1-5 (obtained by mixing F.1-Fr.5 fractions, the same applies hereinafter), Fr.6, Fr.7-11, Fr.12-23, Fr.24-26, Fr.27-29, Fr.30-44, Fr.45-51, Fr.52-67, Fr.68-77, Fr.78-91, Fr.92-99, Fr.100-115, Fr.116-127, Fr.128-137, Fr.138-196, Fr.197-227, Fr.228-258, and Fr.259-275.
(2) The Fr.12-23 supernatants obtained in step (1) were combined, and subjected to Sephadex LH-20 gel column chromatography, using a gel column with a specification of 3×120 cm. In the elution procedure used, methanol was used as a mobile phase for elution, the eluate was collected without interruption from the beginning of the elution procedure according to 30 mL each tube (30 mL/fraction), and 25 fractions were continuously collected, recorded as: tube.1, tube.2, tube.3, . . . , and tube.25. Tubes 5-9 (tube.5 to tube.9 fractions were mixed to give the fraction named tubes 5-9) were separated by preparative high performance liquid chromatography (separated by preparative RP-C18 liquid chromatography). The filler used for the preparative high performance liquid chromatography separation was octadecylsilane bonded silica gel with a particle size of 5 μm. The chromatographic column used for the preparative high performance liquid chromatography separation had a diameter of 8 mm, and a height of 250 mm. The mobile phase was a mixed liquid composed of methanol and water (containing 0.05% formic acid), and the volume ratio of methanol to water in the mobile phase was 90:10; the flow rate was 4.0 mL/min. The elution peaks at t_{R} (retention time) of 16.0 min, 18.0 min, and 19.9 min were collected, and methanol and water were removed by rotary evaporation using a rotary evaporator (temperature: 50° C.) to obtain 80.5 mg of compound C3, 39.1 mg of compound C4, and 4.1 mg of compound C5 sequentially.
(3) The Fr.92-99 obtained in step (1) were subjected to MCI column chromatography, using an MCI column with a specification of 2×30 cm. In the elution procedure used, a mixed liquid composed of methanol and water and having a volume ratio of 90:10 was used as a mobile phase for elution, the eluate was collected without interruption from the beginning of the elution procedure according to 40 mL each tube (40 mL/fraction), and 20 fractions were continuously collected, recorded as: tube.1, tube.2, tube.3, . . . , and tube.20. Tubes 5-9 (obtained by mixing tube.5 to tube.9 fractions) were then subjected to Sephadex LH-20 gel column chromatography, using a gel column with a specification of 2×80 cm. In the elution procedure used, methanol was used as a mobile phase for elution, the eluate was collected without interruption from the beginning of the elution procedure according to 20 mL each tube (20 mL/fraction), and 25 fractions were continuously collected, recorded as: tube.1′, tube.2′, tube.3′, . . . , and tube.25′. Tubes 2′-5′ (obtained by mixing tube.2′ to tube.5′ fractions) were separated by preparative high performance liquid chromatography (separated by preparative RP-C18 liquid chromatography). The filler used for the preparative high performance liquid chromatography separation was octadecylsilane bonded silica gel with a particle size of 5 μm. The chromatographic column used for the preparative high performance liquid chromatography separation had a diameter of 8 mm, and a height of 250 mm. The mobile phase was a mixed liquid composed of methanol and water (containing 0.05% formic acid), and the volume ratio of methanol to water in the mobile phase was 85:15; the flow rate was 4.0 m/min. The elution peaks at t_{R} (retention time) of 11.8 min, 12.5 min, and 17.7 min were collected, and methanol and water were removed by rotary evaporation using a rotary evaporator (temperature: 50° C.) to obtain 21.6 mg of compound C9, 10.6 mg of compound C10, and 102.9 mg of compound C6 sequentially. Tubes 11-15 (obtained by mixing tube.11 to tube. 15 fractions) were then subjected to Sephadex LH-20 gel column chromatography, using a gel column with a specification of 2×80 cm. In the elution procedure used, methanol was used as a mobile phase for elution, the eluate was collected without interruption from the beginning of the elution procedure according to 20 mL each tube (20 mL/fraction), and 15 fractions were continuously collected, recorded as: tube.1″, tube.2″, tube.3″, . . . , and tube.15″. Tubes 2″-8″ (obtained by mixing tube.2″ to tube.8″ fractions) were separated by preparative high performance liquid chromatography (separated by preparative RP-C18 liquid chromatography). The filler used for the preparative high performance liquid chromatography separation was octadecylsilane bonded silica gel with a particle size of 5 μm. The chromatographic column used for the preparative high performance liquid chromatography separation had a diameter of 8 mm, and a height of 250 mm. The mobile phase was a mixed liquid composed of methanol and water (containing 0.05% formic acid), and the volume ratio of methanol to water in the mobile phase was 87:13; the flow rate was 4.0 mE/min. The elution peaks at t_{R} (retention time) of 17.2 min, 19.4 min, and 21.1 min were collected, and methanol and water were removed by rotary evaporation using a rotary evaporator (temperature: 50° C.) to obtain 40.5 mg of compound C6, 28.2 mg of compound C8, and 425.6 mg of compound C7 sequentially.

The Fr.228-258 obtained in step (1) were subjected to MCI column chromatography, using an MCI column with a specification of 6×18 cm. In the elution procedure used, a mixed liquid composed of methanol and water and having a volume ratio of 80:20 was used as a mobile phase for elution, the eluate was collected without interruption from the beginning of the elution procedure according to 100 mL each tube (100 mL/fraction), and 39 fractions were continuously collected, recorded as: tube.1, tube.2, tube.3, . . . , and tube.39. Tubes 16-39 (obtained by mixing tube.16 to tube.39 fractions) were then subjected to ODS column chromatography, using an ODS column with a specification of 5.5×28 cm. In the elution procedure used, a mixed liquid composed of methanol and water and having a volume ratio of 75:25 was used as a mobile phase for elution, the eluate was collected without interruption from the beginning of the elution procedure according to 100 mL each tube (100 mL/fraction), and 50 fractions were continuously collected, recorded as: tube.1′, tube.2′, tube.3′, . . . , and tube.50′. For tubes 34-45 (obtained by mixing tube.34′ to tube.55′ fractions), methanol and water were removed by rotary evaporation using a rotary evaporator (temperature: 50° C.) to obtain 1.25 g of compound C1. Tubes 31-33 (obtained by mixing tube.31′ to tube.33′ fractions) were separated by preparative high performance liquid chromatography (separated by preparative RP-C18 liquid chromatography). The filler used for the preparative high performance liquid chromatography separation was octadecylsilane bonded silica gel with a particle size of 5 μm. The chromatographic column used for the preparative high performance liquid chromatography separation had a diameter of 8 mm, and a height of 250 mm. The mobile phase was a mixed liquid composed of methanol and water (containing 0.05% formic acid), and the volume ratio of methanol to water in the mobile phase was 73:27; the flow rate was 4.5 mL/min. The elution peak at t_{R} (retention time) of 14.0 min was collected, and methanol and water were removed by rotary evaporation using a rotary evaporator (temperature: 50° C.) to obtain 14.4 mg of compound C11.

The Fr.30-44 obtained in step (1) were subjected to MCI column chromatography, using an MCI column with a specification of 3×23 cm. In the elution procedure used, a mixed liquid composed of methanol and water and having a volume ratio of 90:10 was used as a mobile phase for elution, the eluate was collected without interruption from the beginning of the elution procedure according to 50 mL each tube (50 mL/fraction), and 20 fractions were continuously collected, recorded as: tube.1, tube.2, tube.3, . . . , and tube.20. Tubes 7-20 (obtained by mixing tube.7 to tube.20 fractions) were then subjected to Sephadex LH-20 gel column chromatography, using a gel column with a specification of 2×80 cm. In the elution procedure used, methanol was used as a mobile phase for elution, the eluate was collected without interruption from the beginning of the elution procedure according to 20 mL each tube (20 mL/fraction), and 18 fractions were continuously collected, recorded as: tube.1′, tube.2′, tube.3′, . . . , and tube.18′. Tubes 2′-6′ (obtained by mixing tube.2′ to tube.6′ fractions) were separated by preparative high performance liquid chromatography (separated by preparative RP-C18 liquid chromatography). The filler used for the preparative high performance liquid chromatography separation was octadecylsilane bonded silica gel with a particle size of 5 μm. The chromatographic column used for the preparative high performance liquid chromatography separation had a diameter of 8 mm, and a height of 250 mm. The mobile phase was a mixed liquid composed of methanol and water (containing 0.05% formic acid), and the volume ratio of methanol to water in the mobile phase was 84:16; the flow rate was 4.5 m/min. The elution peaks at t_{R} (retention time) of 20.0 min, 23.7 min, 30.4 min, and 33.7 min were collected, and methanol and water were removed by rotary evaporation using a rotary evaporator (temperature: 50° C.) to obtain 3.2 mg of compound C14, 33.5 mg of compound C13, 152.3 mg of compound C12, and 206.1 mg of compound C2 sequentially.

Compounds C1-C14 are brown colloidal solids, are easily soluble in solvents such as methanol, ethanol, and DMSO, and are insoluble in water. The Rf value of compounds C1-C14 was 0.4-0.8 when the compounds were developed by a chloroform-methanol-water (the volume ratio was 70:15:2) solvent system in silica gel thin layer chromatography, the fluorescent color development was obvious at 254 nm and 365 nm, and the vanillin sulfuric acid color development was brownish red.

### Example 4: Structural Identification

### (1) Compounds C1 and C2

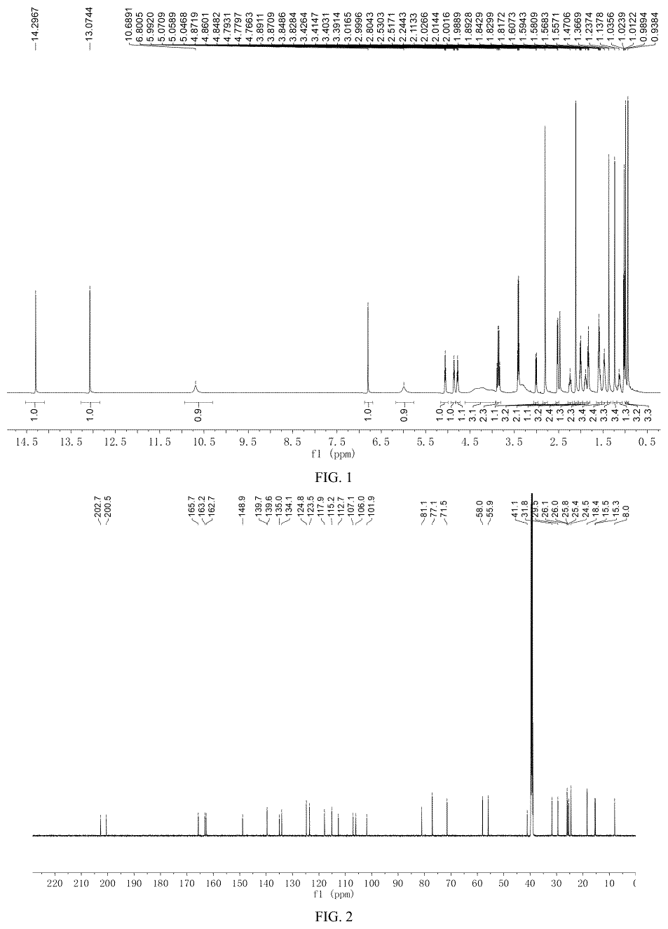

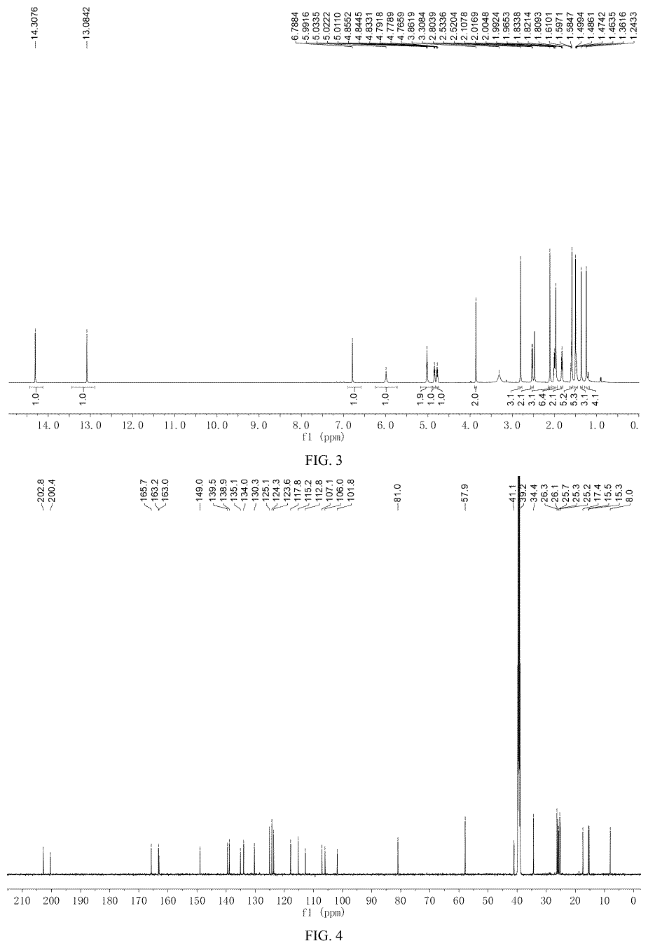

The spectral data of compounds C1 and C2 are shown in Table 1, and the ¹H NMR and ¹³C NMR spectra of compounds C1 and C2 are shown in FIGS. 1-4, respectively. The structures of compounds C1 and C2 are shown in Table 6.

TABLE 1
Nuclear magnetic resonance data of compounds C1
and C2 (¹H NMR 600 MHz; ¹³C NMR 150 MHz; DMSO-d₆)

| | C1 | | | C2 | | |
|---|---|---|---|---|---|---|
| POS. | δ_{C} | δ_{H} (J, Hz) | | δ_{C} | δ_{H} (J, Hz) | |
| 1 | 81.2 | / | | 81.2 | / | |
| 2 | 200.7 | / | | 200.6 | / | |
| 3 | 102.0 | / | | 101.9 | / | |
| 4 | 135.2 | / | | 135.2 | / | |
| 5 | 106.1 | / | | 106.1 | / | |
| 6 | 202.9 | / | | 202.9 | / | |
| 7 | 163.3 | / | | 163.3 | / | |
| 8 | 118.0 | 6.80 | s | 118.0 | 6.79 | s |
| 9 | 149.0 | / | | 149.1 | / | |
| 10 | 112.8 | / | | 112.9 | / | |
| 11 | 162.9 | / | | 163.2 | / | |
| 12 | 107.2 | / | | 107.2 | / | |
| 13 | 165.8 | / | | 165.8 | / | |
| 14 | 8.1 | 2.11 | s | 8.1 | 2.11 | s |
| 15 | 26.2 | 2.80 | s | 26.2 | 2.81 | s |
| 16 | 41.3 | 2.52 | d (7.9) | 41.2 | 2.52 | d (7.9) |
| 17 | 115.3 | 4.78 | t (8.0) | 115.3 | 4.77 | t (7.7) |
| 18 | 139.7 | / | | 139.7 | / | |
| 19 | 39.2 | 1.59 | t (7.9) | 39.2 | 1.59 overlap | |
| 20 | 25.9 | 1.47 | m | 25.8 | 1.48 | m |
| 21 | 123.7 | 4.85 | t (7.1) | 123.8 | 4.84 | t (6.4) |
| 22 | 134.3 | / | | 134.1 | / | |
| 23 | 39.4 | 1.83 | t (7.6) | 39.4 | 1.82 | t (7.4) |
| 24 | 25.5 | 2.00 | dd (15.0, 7.3) | 25.3 | 2.00 | dd (14.7, 7.3) |
| 25 | 124.9 | 5.06 | t (7.2) | 125.2 | 5.02 | t (6.8) |
| 26 | 139.8 | / | | 139.0 | / | |
| 27 | 31.9 | 2.24 m, 1.89 m | | 34.5 | 1.96 | m |
| 28 | 29.6 | 1.57 m, 1.12 m | | 26.4 | 1.98 | m |
| 29 | 77.2 | 3.00 | d (10.1) | 124.4 | 5.02 | t (6.8) |
| 30 | 71.6 | / | | 134.0 | / | |
| 31 | 26.1 | 0.99 | s | 25.5 | 1.58 | s |
| 32 | 24.6 | 0.94 | s | 17.5 | 1.50 | s |
| 33 | 58.2 | 3.85 | dd (25.0, 12.1) | 58.0 | 3.86 | s |
| 34 | 15.3 | 1.39 | s | 15.4 | 1.36 | s |
| 35 | 15.6 | 1.24 | s | 15.6 | 1.24 | s |
| 7-OH | / | 13.07 | s | / | 13.08 | s |
| 13-OH | / | 14.30 | s | / | 14.30 | s |

### (2) Compound C3

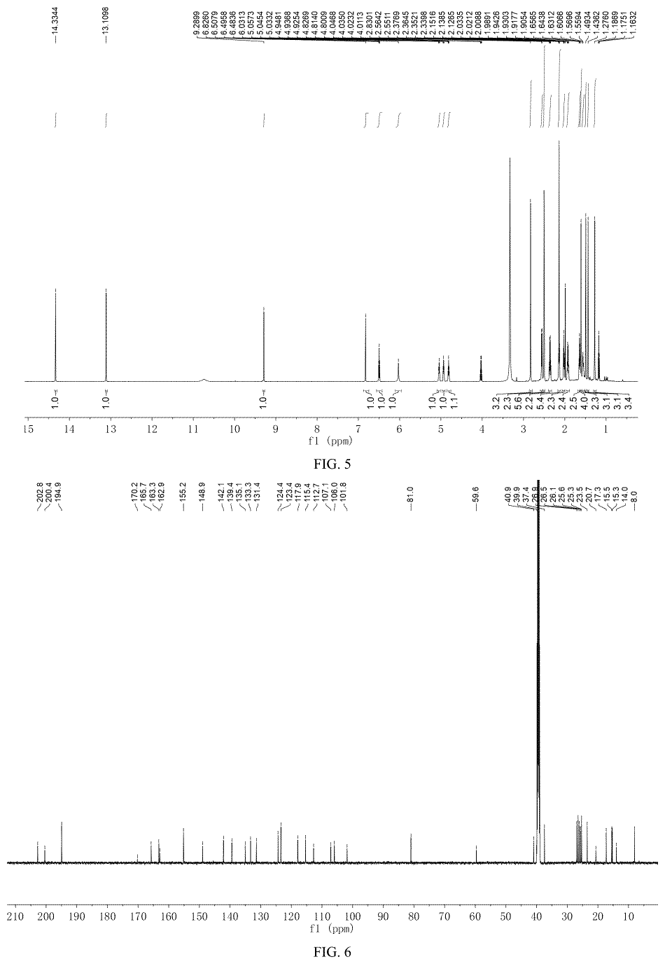

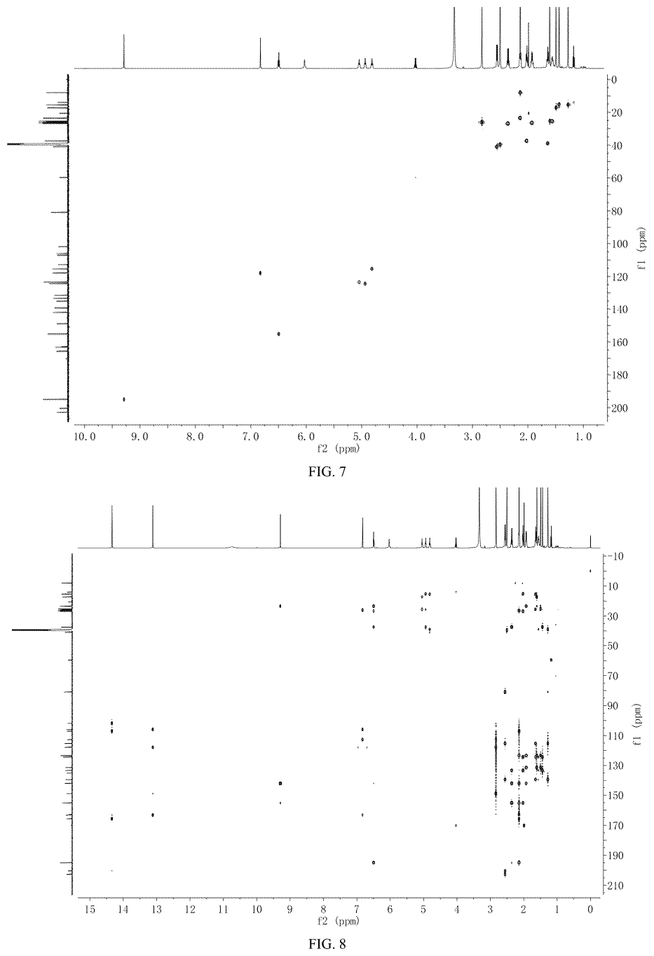

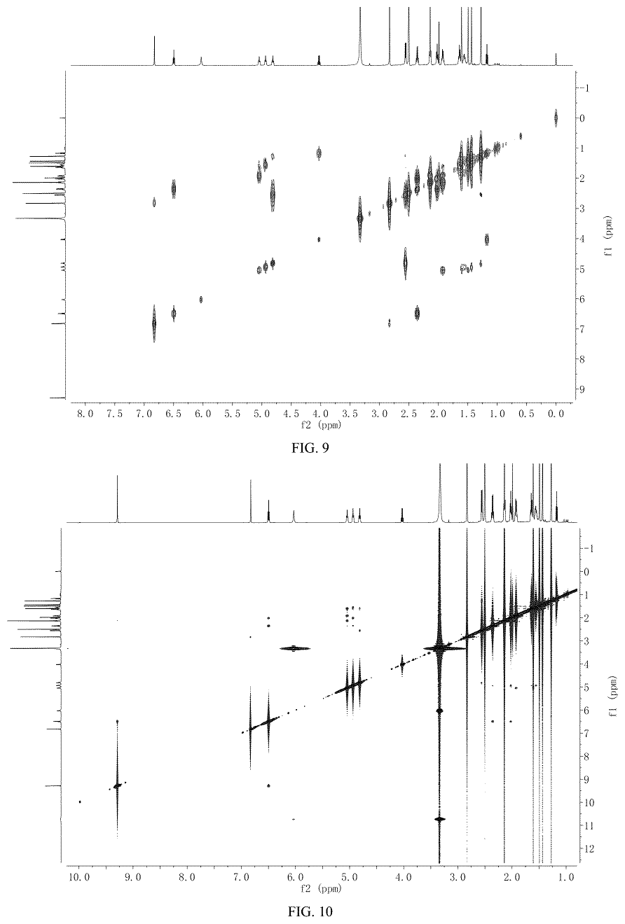

HRESIMS (negative ion) ion peak of compound C3: m/z 573.2825[M-H⁻]⁻, indicating that its molecular formula is C₃₅H₄₂O₇. According to the comprehensive analysis of ¹H NMR (FIG. 5), ¹³C NMR (FIG. 6), and HSQC spectrum (FIG. 7), it was presumed that the compound should contain 2 ketone carbonyl, 1 aldehyde carbonyl, 18 alkene carbons, and 14 sp³ hybridized carbons. The characteristic signals were found in the hydrogen spectrum: δ 14.33 (1H, s), 13.11 (1H, s), 9.29 (1H, s), 6.83 (1H, s), 6.50 (1H, t, J=7.3 Hz), 5.05 (1H, t, J=7.2 Hz), 4.94 (1H, t, J=6.8 Hz), 4.81 (1H, t, J=7.8 Hz), 2.83 (3H, s), 2.56 (2H, d, J=7.9 Hz), 2.14 (3H, s), 1.57 (3H, s), 1.49 (3H, s), 1.44 (3H, s), 1.28 (3H, s). The above characteristic signals are similar to those of compound C2, and a detailed comparison between the nuclear magnetic resonance signals of compounds C3 and C2 reveals that both have the same perinaphthenone three-membered ring structure, differing only in the diterpene branched chain. The following associated signals can be found in the HMBC spectrum (FIG. 8): δ1.28 (H-35) is associated with δ115.4 (C-17), 139.4 (C-18), and 39.2 (C-19), δ1.44 (H-34) is associated with 124.4 (C-21), 133.3 (C-22), and 37.4 (C-23), δ9.29 (H-33) is associated with δ155.2 (C-25), 142.1 (C-26), and 23.5 (C-27), δ5.05 (H-29) is associated with δ23.5 (C-27), 25.3 (C-31), and 17.3 (C-32), δ6.50 (H-25) is associated with δ26.9 (C-24), 23.5 (C-27), and 194.9 (C-33), δ4.94 (H-21) is associated with δ38.2 (C-19), 37.4 (C-23), and 15.3 (C-34), and 64.81 (H-17) is associated with δ39.2 (C-19), and 15.5 (C-35). The following associated signals can be found in the ¹H-¹H COSY spectrum (FIG. 9): δ4.81 (H-17) is associated with δ2.56 (H-16), δ4.94 (H-21) is associated with δ1.56 (H-20), δ1.56 (H-20) is associated with δ1.64 (H-19), δ6.50 (H-25) is associated with δ2.36 (H-24), δ2.36 (H-24) is associated with δ2.02 (H-23), δ5.05 (H-29) is associated with δ1.93 (H-28), and 61.93 (H-28) is associated with δ2.14 (H-27). The HMBC and ¹H-¹H COSY associated signals described above ensure that the plane structure of the diterpene branched chain is determined. The following associated signals can be found in the NOESY spectrum (FIG. 10): δ4.81 (H-17) is associated with δ1.64 (H-19), δ4.94 (H-21) is associated with δ2.02 (H-23), and 66.50 (H-25) is associated with δ9.29 (H-33), thus demonstrating that the configuration of 17(18)-ene, 21(22)-ene, and 25(26)-ene are E−, E−, and E−, respectively. Considering that compound C3 has the same biosynthetic pathway as that of compounds C1 and C2, the absolute configuration of position C-1 of compound C3 should also be S configuration. Finally, compound C3 was identified as having a structure shown in Table 6, and its nuclear magnetic resonance signal assignments are specifically shown in Table 2.

### (3) Compound C4

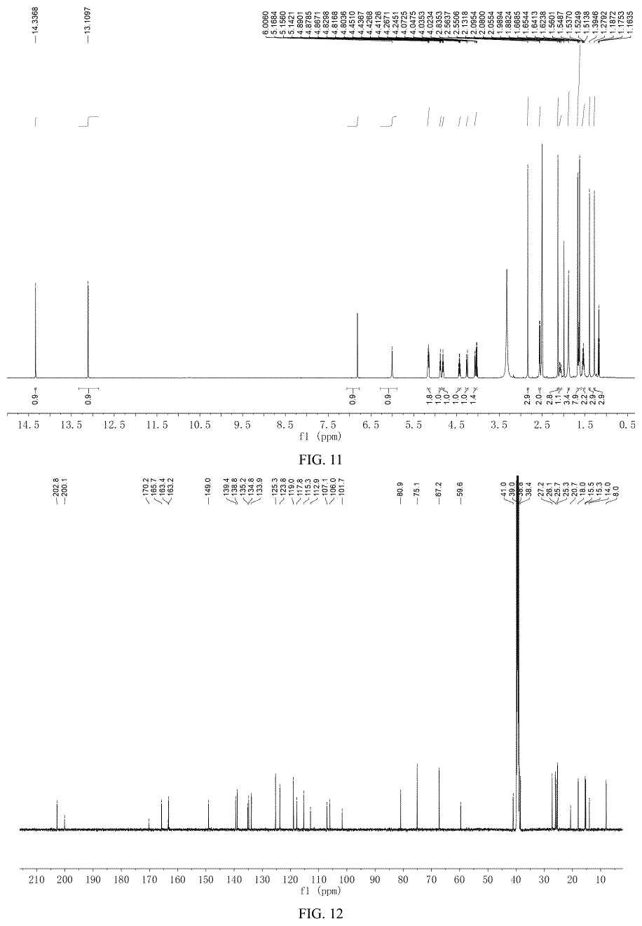

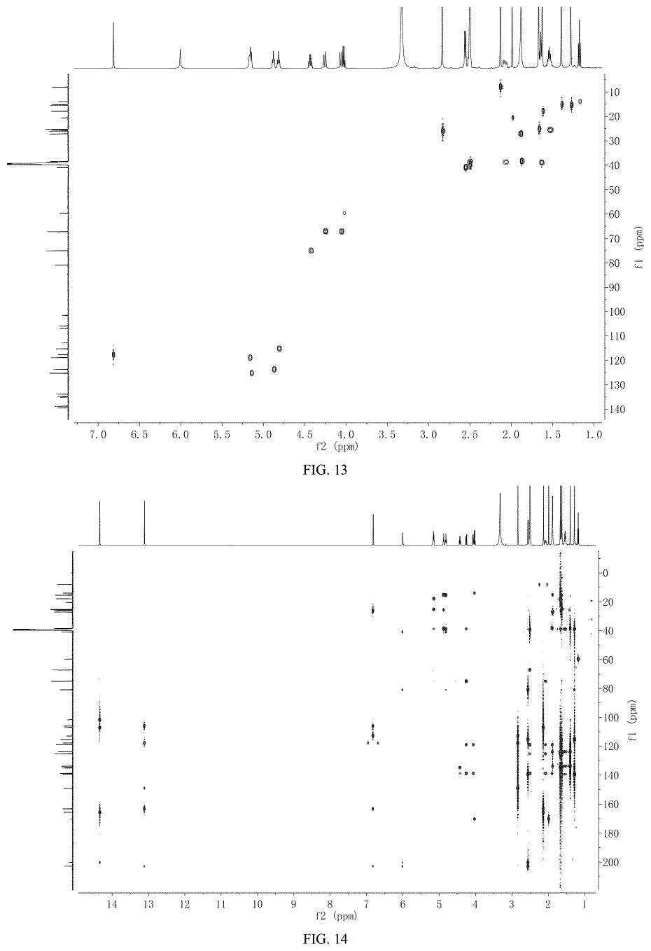

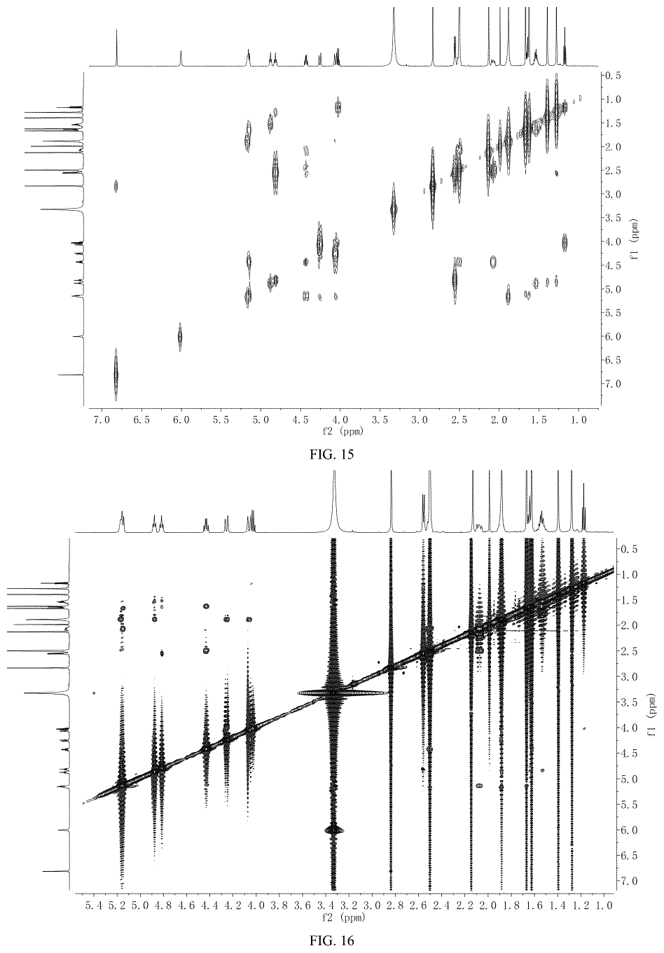

HRESIMS (negative ion) ion peak of compound C4: m/z 573.2838[M-H]⁻, indicating that its molecular formula is C₃₅H₄₂O₇. According to the comprehensive analysis of ¹H NMR (FIG. 11), ¹³C NMR (FIG. 12), and HSQC spectrum (FIG. 13), it was presumed that the compound should contain 2 ketone carbonyl, 18 alkene carbons, and sp³ hybridized carbons. The nuclear magnetic resonance signal of compound C4 is substantially the same as the nuclear magnetic resonance signal of compound C2, differing only in C-25 to C-28 structural fragments. In the ¹H-¹H COSY spectrum (FIG. 15), it could be seen that H-29 is associated with H-28, and in combination with the HSQC associated signal, the signals δH4.43 and δ_{C}75.1 are assigned to position C-28. C-28 was determined to be oxidized according to the chemical shift values of the signal. The following associated signals can be found in the HMBC spectrum (FIG. 14): H-25 (δ4.26, 4.06) is associated with C-25 (δ119.0), and C-26 (δ138.8), and H-25 (δ4.26, 4.06) is associated with C-28 (δ75.1), indicating that C-28 and C-33 are linked by an ether bond. In the NOESY spectrum (FIG. 16), δ5.17 (H-25) is associated with δ2.50 (H-27), demonstrating that the configuration of 25(26)-ene is E−. Considering that compound C4 has the same biosynthetic pathway as that of compounds C1 and C2, the configuration of position C-1 of compound C4 is S configuration. Finally, compound C4 was identified as having the structure shown in Table 6, and its nuclear magnetic resonance signal assignments are specifically shown in Table 2.

### (4) Compound C5

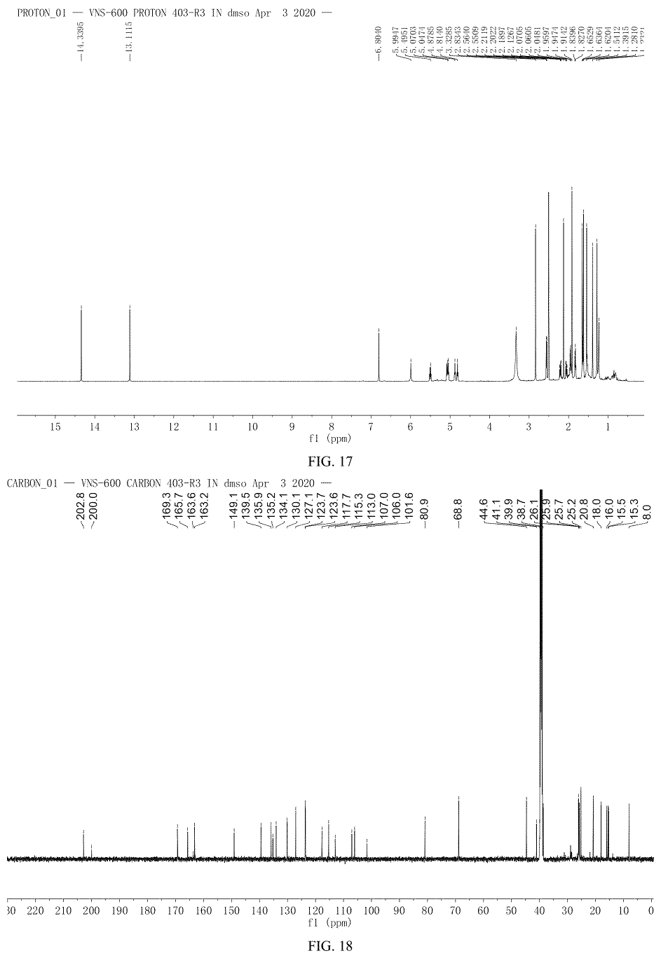

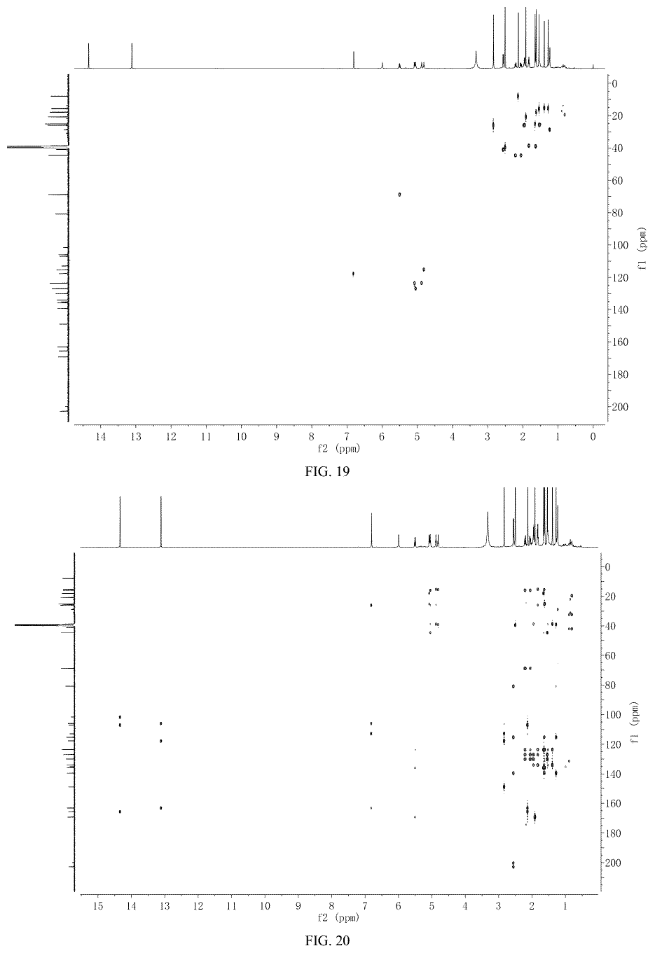

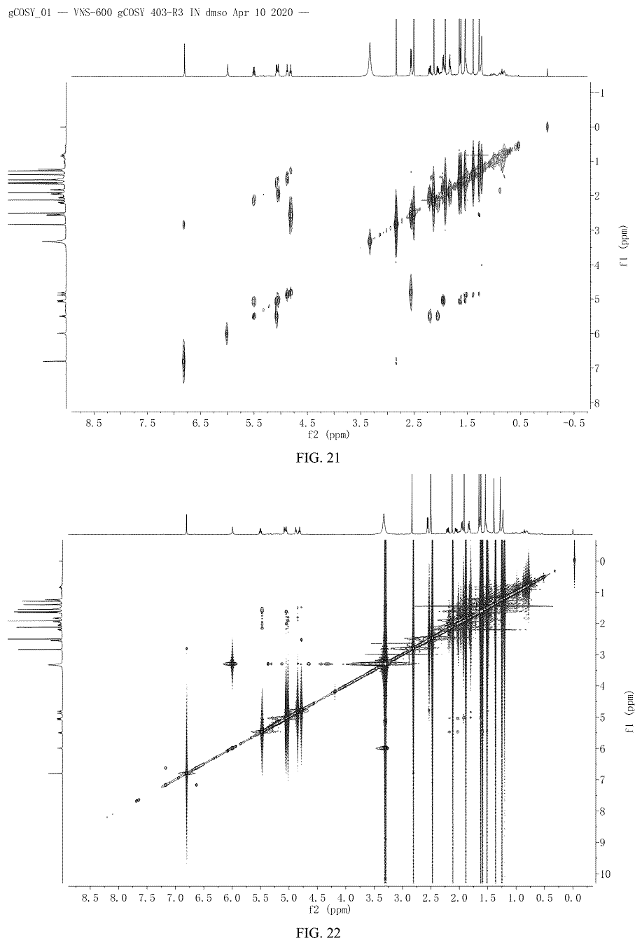

HRESIMS (negative ion) ion peak of compound C5: m/z 617.3372[M-H⁻]⁻, indicating that its molecular formula is C₃₇H₄₆O₈. According to the comprehensive analysis of ¹H NMR (FIG. 17), ¹³C NMR (FIG. 18), and HSQC spectrum (FIG. 19), it was presumed that the compound should contain 2 ketone carbonyl, 1 ester carbonyl, 18 alkene carbons, and 16 sp³ hybridized carbons. The nuclear magnetic resonance signal of compound C5 is similar to the nuclear magnetic resonance signal of compound C2 in most parts, differing in C-25 to C-30 structural fragments. In the HMBC spectrum (FIG. 20), it could be seen that 61.54 (H-33) is associated with 106.0 (C-5), 130.1 (C-26), and 44.6 (C-27), δ1.62 (H-31) is associated with 123.7 (C-29), 135.9 (C-30), and 25.2 (C-31), and δ1.65 (H-32) is associated with δ123.7 (C-29), 135.9 (C-30), and 18.0 (C-32). In the ¹H-¹H COSY spectrum (FIG. 21), it could be seen that 65.50 (H-28) is associated with δ2.2, 2.06 (H-27), and 5.07 (H-29), respectively. According to the evidence described above, the framework structures of C-25 to C-30 could be determined. In the HMBC spectrum, it could be seen that 65.50 (H-28) is associated with δ169.3 (—COCH₃), and 61.91 (—COCH₃) is associated with δ169.3 (—COCH₃), demonstrating that one acetyl is connected to position C-28. The following associated signals can be found in the NOESY spectrum (FIG. 22): δ4.82 (H-17) is associated with δ1.64 (H-19), δ4.87 (H-21) is associated with δ1.83 (H-23), and 65.05 (H-25) is associated with δ2.20 (H-27), thus demonstrating that the configuration of 17(18)-ene, 21(22)-ene, and 25(26)-ene are E−, E−, and E−, respectively. Considering that compound C5 has the same biosynthetic pathway as that of compounds C1 and C2, the configuration of position C-1 of compound C5 should also be S configuration. Finally, compound C5 was identified as having the structure shown in Table 6, and its nuclear magnetic resonance signal assignments are specifically shown in Table 2.

TABLE 2
Nuclear magnetic resonance data of compounds C3
to C5 (¹H NMR 600 MHz; ¹³C NMR 150 MHz; DMSO-d₆)

| | C3 | | | C4 | | | C5 | | |
|---|---|---|---|---|---|---|---|---|---|
| POS. | δ_{C} | δ_{H} (J, Hz) | | δ_{C} | δ_{H} (J, Hz) | | δ_{C} | δ_{H} (J, Hz) | |
| 1 | 81.0 | / | | 80.9 | / | | 80.9 | / | |
| 2 | 200.4 | / | | 200.1 | / | | 200.0 | / | |
| 3 | 101.8 | / | | 101.7 | / | | 101.6 | / | |
| 4 | 135.1 | / | | 135.2 | / | | 135.2 | / | |
| 5 | 106.0 | / | | 106.0 | / | | 106.0 | / | |
| 6 | 202.8 | / | | 202.8 | / | | 202.8 | / | |
| 7 | 163.3 | / | | 163.4 | / | | 163.2 | / | |
| 8 | 117.9 | 6.83 | s | 117.8 | 6.81 | s | 117.7 | 6.81 | s |
| 9 | 149.0 | / | | 149.0 | / | | 149.1 | / | |
| 10 | 112.7 | / | | 112.9 | / | | 113.0 | / | |
| 11 | 162.9 | / | | 163.2 | / | | 163.6 | / | |
| 12 | 107.1 | / | | 107.1 | / | | 107.0 | / | |
| 13 | 165.8 | / | | 165.7 | / | | 165.7 | / | |
| 14 | 8.0 | 2.14 | s | 8.0 | 2.13 | s | 8.0 | 2.13 | s |
| 15 | 26.1 | 2.83 | s | 26.1 | 2.83 | s | 26.1 | 2.83 | s |
| 16 | 40.9 | 2.56 | d (7.9) | 41.0 | 2.56 | d (7.9) | 41.1 | 2.56 | d (7.9) |
| 17 | 115.4 | 4.81 | t (7.8) | 115.3 | 4.82 | t (7.9) | 115.3 | 4.82 | t (7.8) |
| 18 | 139.4 | / | | 139.4 | / | | 139.5 | / | |
| 19 | 39.2 | 1.64 | t (7.6) | 39.0 | 1.64 overlap | | 39.1 | 1.64 overlap | |
| 20 | 25.6 | 1.56 | m | 25.7 | 1.53 | m | 25.9 | 1.96 | m |
| 21 | 124.4 | 4.94 | t (6.8) | 123.8 | 4.87 | t (6.9) | 123.6 | 4.87 | t (6.8) |
| 22 | 133.3 | / | | 133.9 | / | | 134.1 | / | |
| 23 | 37.4 | 2.02 | t (7.4) | 38.4 | 1.88 overlap | | 38.7 | 1.83 overlap | |
| 24 | 26.9 | 2.36 | dd (14.9, 7.4) | 27.2 | 1.88 overlap | | 25.7 | 1.53 | m |
| 25 | 155.2 | 6.50 | t (7.3) | 119.0 | 5.17 overlap | | 127.1 | 5.05 | t (7.4) |
| 26 | 142.1 | / | | 138.8 | / | | 130.1 | / | |
| 27 | 23.5 | 2.14 overlap | | 38.8 | 2.50 m, 2.07 m | | 44.6 | 2.20 m, 2.06 m | |
| 28 | 26.5 | 1.93 dd | (14.9, 7.4) | 75.1 | 4.43 | m | 68.8 | 5.50 | q (7.2) |
| 29 | 123.4 | 5.05 | t (7.2) | 125.3 | 5.25 overlap | | 123.7 | 5.07 | d (9.0) |
| 30 | 131.4 | / | | 134.8 | / | | 135.9 | / | |
| 31 | 25.3 | 1.61 | s | 25.3 | 1.67 | s | 18.0 | 1.62 | s |
| 32 | 17.3 | 1.49 | s | 18.0 | 1.62 | s | 25.2 | 1.65 | s |
| 33 | 194.9 | 9.29 | s | 67.2 | 4.26 | d (13.2) | 16.0 | 1.54 | s |
| | | | | | 4.06 | d (15.0) | | | |
| 34 | 15.3 | 1.44 | s | 15.3 | 1.39 | s | 15.3 | 1.39 | s |
| 35 | 15.5 | 1.28 | s | 15.5 | 1.28 | s | 15.5 | 1.28 | s |
| 7—OH | / | 13.11 | s | / | 13.10 | s | / | 13.11 | s |
| 13—OH | / | 14.33 | s | / | 14.34 | s | / | 14.33 | s |

| | | | | | | |
|---|---|---|---|---|---|---|
| C═O | / | / | / | / | / | 169.3 |
| CH₃₋ | / | / | / | / | / | 20.8 |

### (5) Compound C6

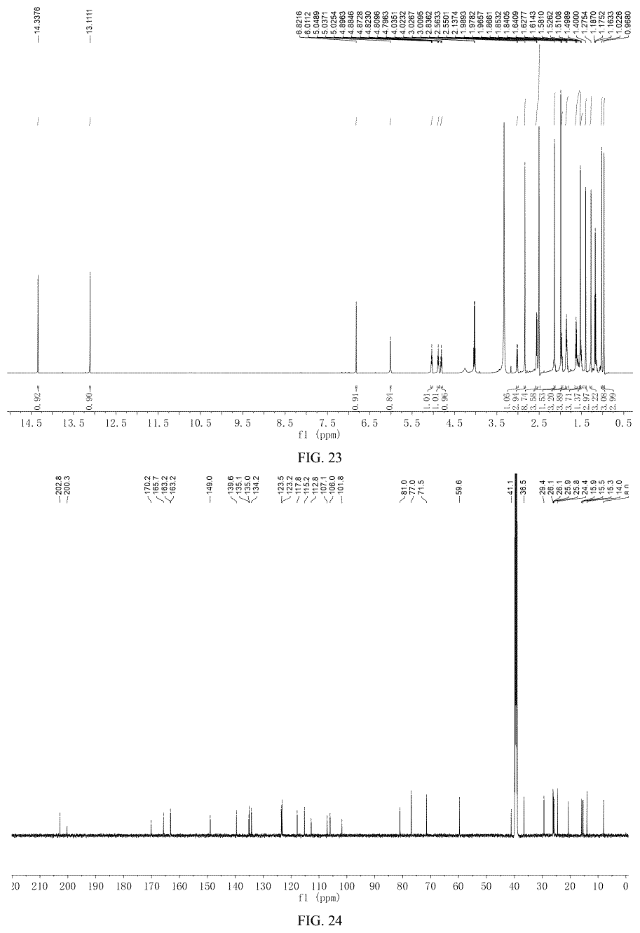

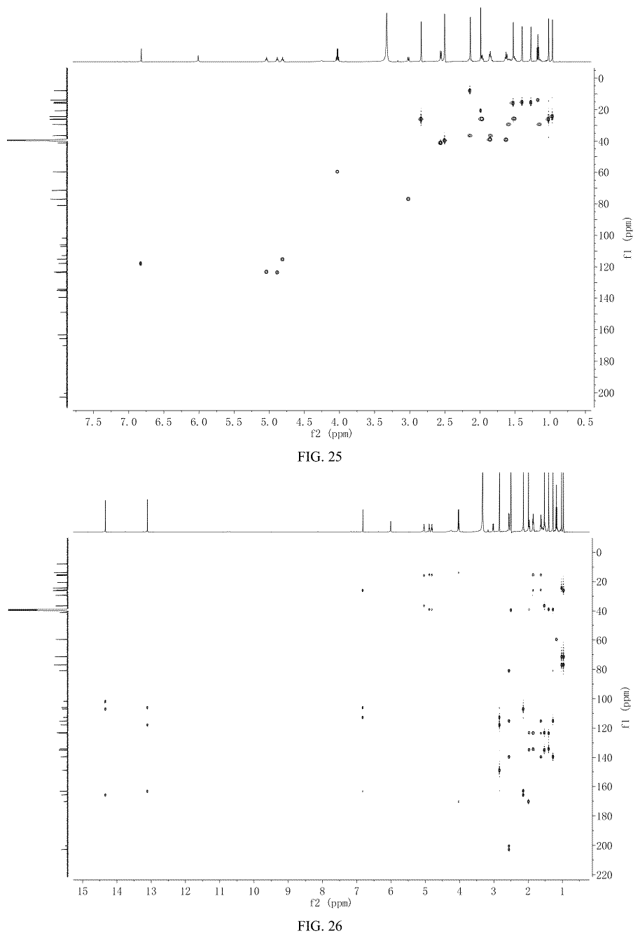

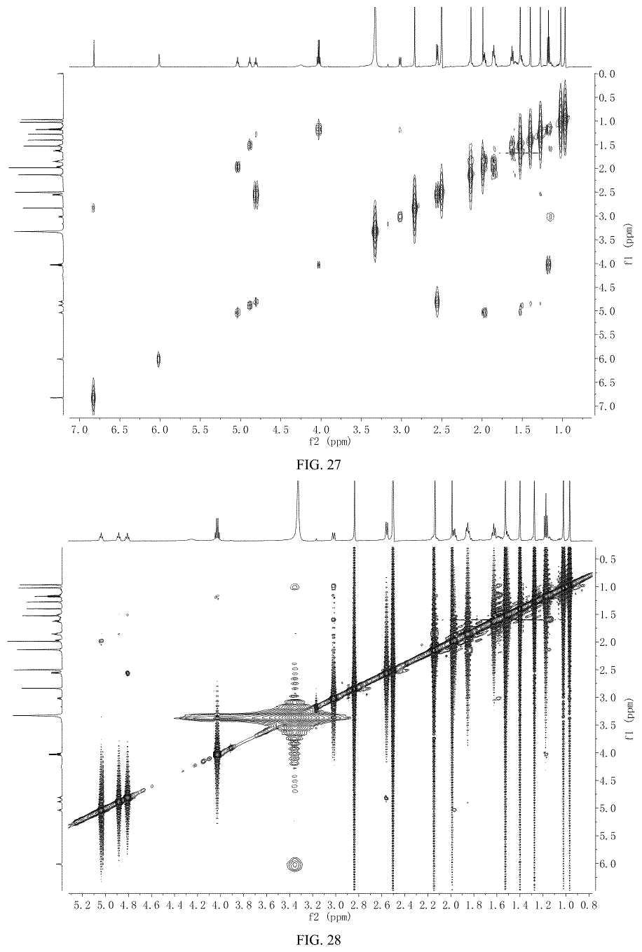

HRESIMS (negative ion) ion peak of compound C6: m/z 593.31 15[M-H⁻]⁻, indicating that its molecular formula is C₃₅H₄₆O₈. According to the comprehensive analysis of ¹H NMR (FIG. 23), ¹³C NMR (FIG. 24), and HSQC spectrum (FIG. 25), it was presumed that the compound should contain 2 ketone carbonyl, 16 alkene carbons, and 17 sp³ hybridized carbons. The nuclear magnetic resonance signal of compound C6 is substantially the same as the nuclear magnetic resonance signal of compound C1, differing only in position C-26. The following associated signals can be found in the HMBC spectrum (FIG. 26): δ5.04 (H-25) is associated with δ36.5 (C-27) and 15.9 (C-33), 31.53 (H-33) is associated with δ123.2 (C-25), 135.0 (C-26), and 36.5 (C-27), indicating a methyl substitution at position C-26. In the NOESY spectrum (FIG. 28), δ5.04 (H-25) is associated with δ1.85 (H-27), demonstrating that the configuration of 25(26)-ene is E−. Considering that compound C6 has the same biosynthetic pathway as that of compounds C1 and C2, the configurations of positions C-1 and C-29 of compound C6 are S and R configuration, respectively. Finally, compound C6 was identified as having a structure shown in Table 6, and its nuclear magnetic resonance signal assignments are specifically shown in Table 3.

### (6) Compound C7

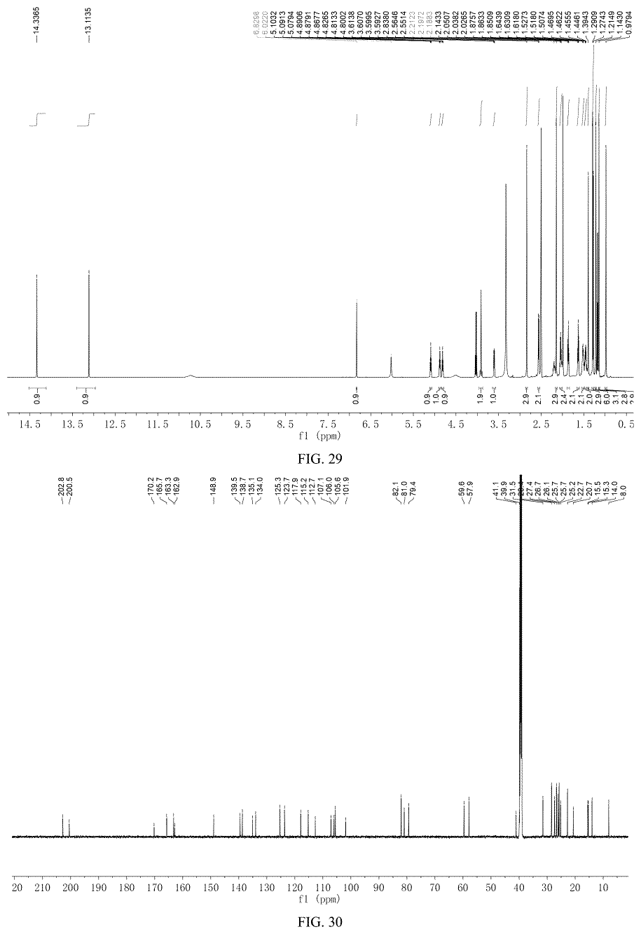

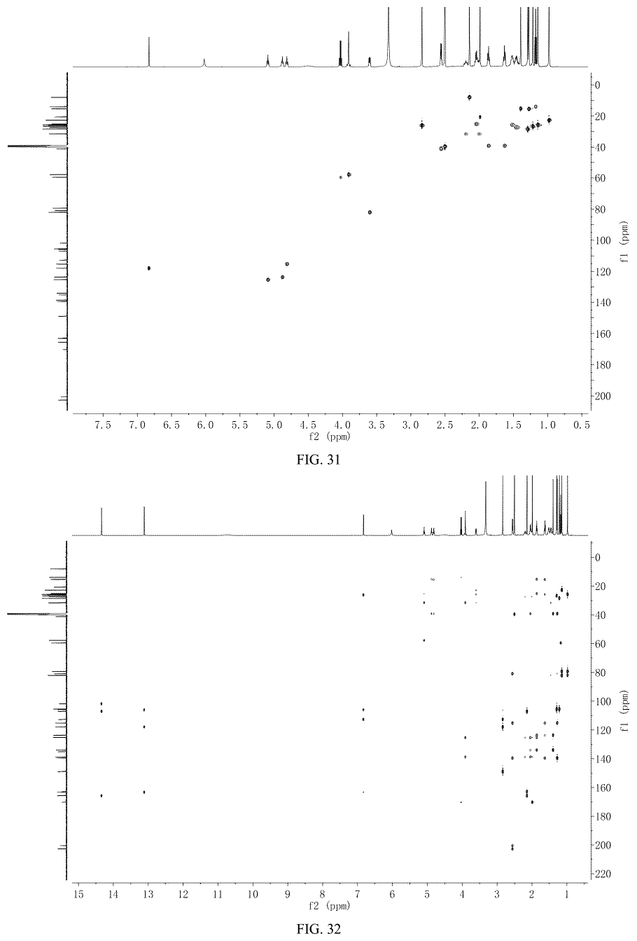

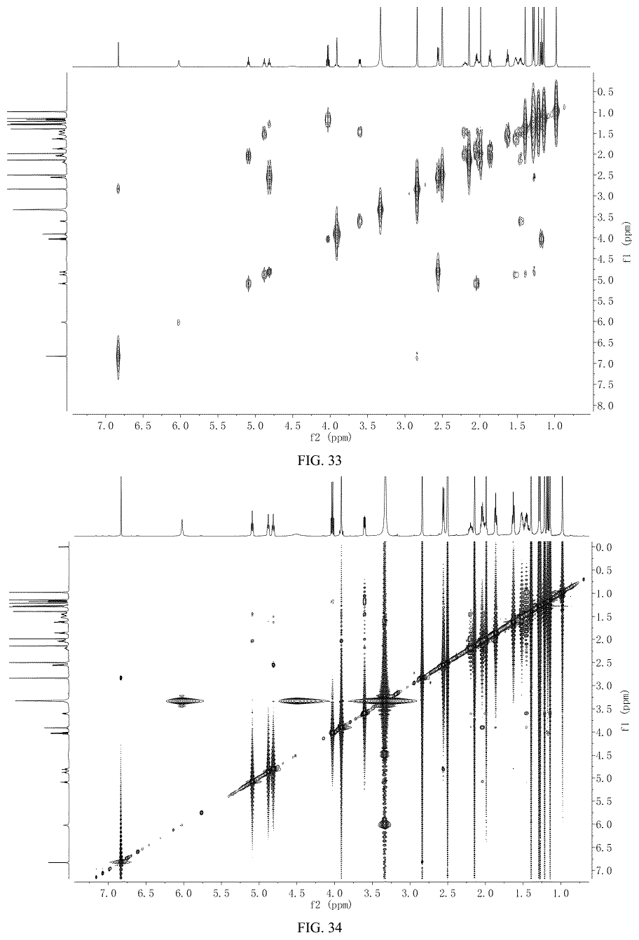

HRESIMS (negative ion) ion peak of compound C7: m/z 649.3383[M-H⁻]⁻, indicating that its molecular formula is C₃₈H₅₀O₉. According to the comprehensive analysis of ¹H NMR (FIG. 29), ¹³C NMR (FIG. 30), and HSQC spectrum (FIG. 31), it was presumed that the compound should contain 2 ketone carbonyl, 16 alkene carbons, and 20 sp³ hybridized carbons. The nuclear magnetic resonance signal of compound C7 is substantially the same as the nuclear magnetic resonance signal of compound C1, differing only in C-28 to C-32 structural fragments on the diterpene branched chain. Compound C7 has three more carbon atoms than compound C1, including one oxidized quaternary carbon δ 105.6 (C-1′), and two methyl carbon signals δ28.4 (C-2′) [δ1.29 (3H,s,H-2′)], δ26.7 (C-3′) [δ1.21 (3H, s, H-3′)]. In the HMBC spectrum (FIG. 46), δ1.29 (H-2′) is associated with δ105.6 (C-1′) and 26.7 (C-3′), and δ1.21 (H-3′) is associated with δ105.6 (C-1′) and 28.4 (C-2′), indicating that C-1′ is substituted with two methyl. Furthermore, the relatively large chemical shift of C-1′ suggests that it should be substituted with two oxygen atoms. HMBC associated signals: δ1.15 (H-31) is associated with δ79.4 (C-30) and 82.1 (C-29), and 630.98 (H-32) is associated with 79.4 (C-30) and 82.1 (C-29), and the structure of C-28 to C-32 fragments was further determined in combination with ¹H-¹H COSY associated signals: δ3.61 (H-29) is associated with δ2.04 (H-28), and δ2.04 (H-28) is associated with δ2.19 (H-27a) and 2.00 (H-27b). According to the relatively large chemical shifts of C-29 and C-30 in combination with the unsaturation of compound C7, C-1′ is linked to C-29 and C-30 via an ether bond, finally forming a 5-membered ring structure fragment. Considering that compound C7 has the same biosynthetic pathway as that of compounds C1 and C2, the absolute configuration of position C-1 of compound C7 should also be S configuration. Finally, compound C7 was identified as having a structure shown in Table 6, and its nuclear magnetic resonance signal assignments are specifically shown in Table 3.

### (7) Compound C8

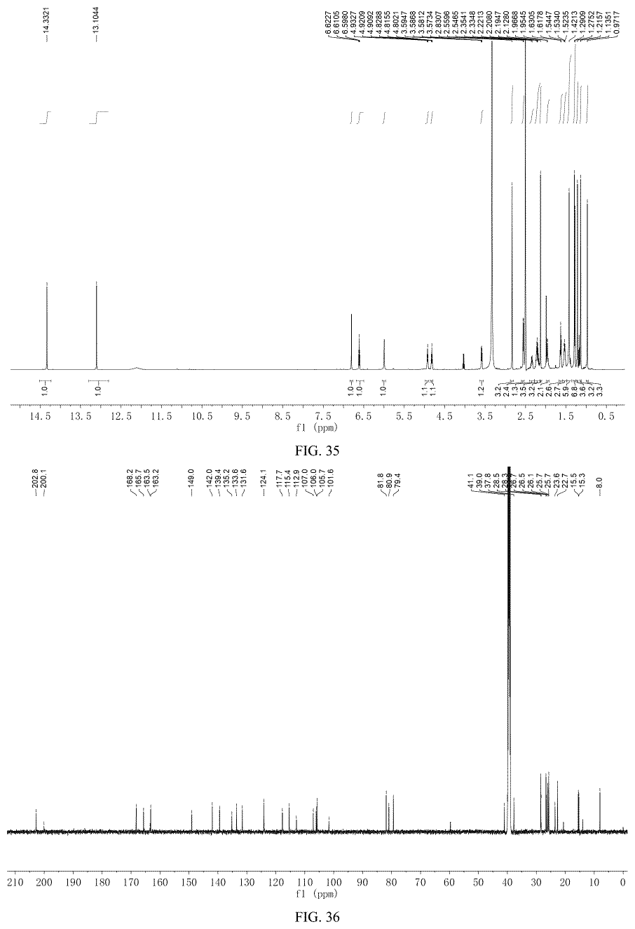

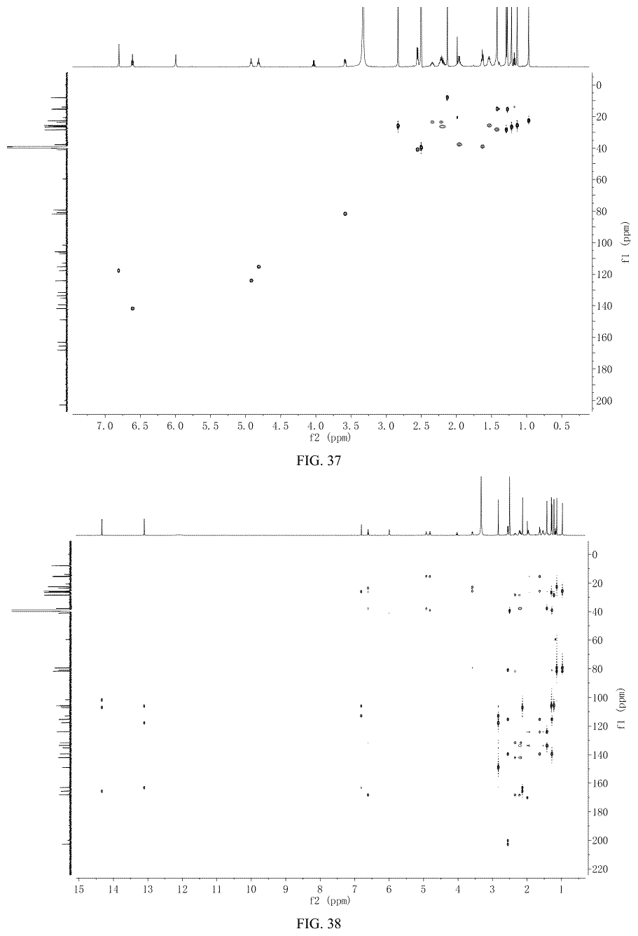

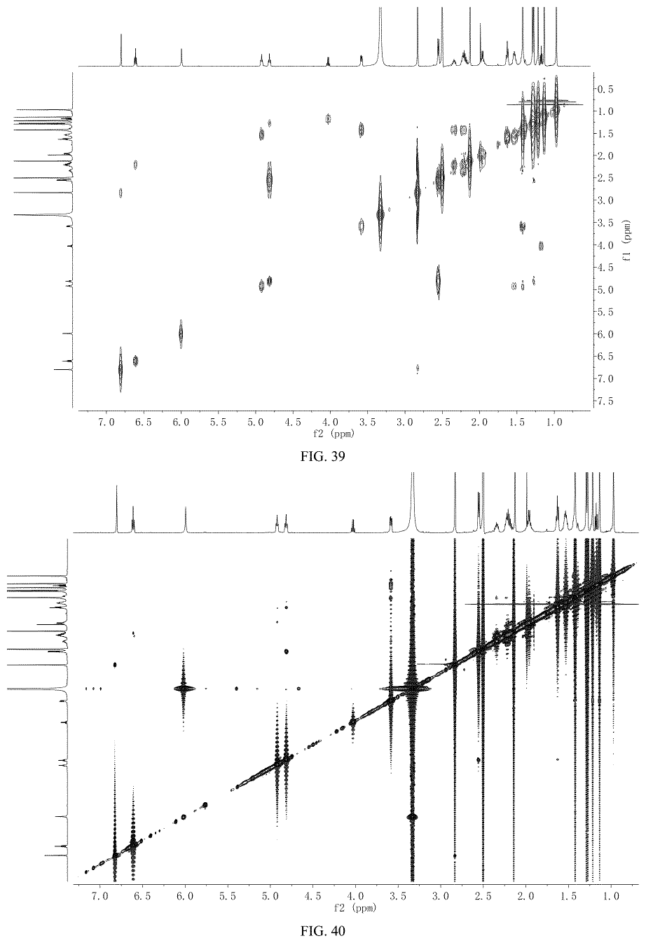

HRESIMS (negative ion) ion peak of compound C8: m/z 663.3114[M-H]⁻, indicating that its molecular formula is C₃₈H₄₈O₁₀. According to the comprehensive analysis of ¹H NMR (FIG. 35), ¹³C NMR (FIG. 36), and HSQC spectrum (FIG. 37), it was presumed that the compound should contain 2 ketone carbonyl, 1 ester carbonyl, 16 alkene carbons, and 19 sp³ hybridized carbons. The nuclear magnetic resonance signal of compound C8 is substantially the same as the nuclear magnetic resonance signal of compound C7, differing only in position C-26 on the diterpene branched chain. In the HMBC spectrum (FIG. 38), δ6.60 (H-25) is associated with δ23.6 (C-27), 37.8 (C-23), and 168.2 (C-33), indicating that position C-26 is substituted with one carboxyl (—COOH). In the NOESY spectrum (FIG. 40), δ2.34 (H-27) is associated with δ2.20 (H-24), demonstrating that the configuration of 25(26)-ene is E−. Considering that compound C8 has the same biosynthetic pathway as that of compounds C1 and C2, the absolute configuration of position C-1 of compound C8 should also be S configuration. Finally, compound C8 was identified as having a structure shown in Table 6, and its nuclear magnetic resonance signal assignments are specifically shown in Table 3.

TABLE 3
Nuclear magnetic resonance data of compounds C6
to C8 (¹H NMR 600 MHz; ¹³C NMR 150 MHz; DMSO-d₆)

| | C6 | | | C7 | | | C8 | | |
|---|---|---|---|---|---|---|---|---|---|
| POS. | δ_{C} | δ_{H} (J, Hz) | | δ_{C} | δ_{H} (J, Hz) | | δ_{C} | δ_{H} (J, Hz) | |
| 1 | 81.0 | / | | 81.0 | / | | 80.9 | / | |
| 2 | 200.3 | / | | 200.5 | / | | 200.1 | / | |
| 3 | 101.8 | / | | 101.9 | / | | 101.6 | / | |
| 4 | 135.1 | / | | 135.1 | / | | 135.2 | / | |
| 5 | 106.0 | / | | 106.0 | / | | 106.0 | / | |
| 6 | 202.8 | / | | 202.8 | / | | 202.8 | / | |
| 7 | 163.2 | / | | 163.3 | / | | 163.5 | / | |
| 8 | 117.8 | 6.82 | s | 117.9 | 6.83 | s | 117.7 | 6.80 | s |
| 9 | 149.0 | / | | 149.0 | / | | 149.0 | / | |
| 10 | 112.8 | / | | 112.7 | / | | 112.9 | / | |
| 11 | 163.2 | / | | 162.9 | / | | 163.2 | / | |
| 12 | 107.1 | / | | 107.1 | / | | 107.0 | / | |
| 13 | 165.7 | / | | 165.7 | / | | 165.7 | / | |
| 14 | 8.0 | 2.14 | s | 8.0 | 2.15 | s | 8.0 | 2.13 | s |
| 15 | 26.1 | 2.84 | s | 26.1 | 2.83 | s | 26.1 | 2.83 | s |
| 16 | 41.1 | 2.56 | d (7.9) | 41.1 | 2.56 | d (7.9) | 41.1 | 2.55 | d (7.9) |
| 17 | 115.2 | 4.81 | t (8.0) | 115.2 | 4.81 | t (7.9) | 115.4 | 4.81 | t (7.9) |
| 18 | 139.6 | / | | 139.5 | / | | 139.4 | / | |
| 19 | 39.1 | 1.63 | t (8.0) | 39.1 | 1.63 | t (7.8) | 39.0 | 1.63 | m |
| 20 | 25.8 | 1.51 | m | 25.7 | 1.52 | m | 25.7 | 1.53 | m |
| 21 | 123.5 | 4.88 | t (7.0) | 123.7 | 4.88 | t (6.9) | 124.1 | 4.92 | t (7.1) |
| 22 | 134.2 | / | | 134.0 | / | | 133.6 | / | |
| 23 | 39.0 | 1.86 | m | 39.1 | 1.86 | t (7.5) | 37.8 | 1.96 | m |
| 24 | 25.9 | 1.97 | dd (14.8, 7.4) | 25.2 | 2.04 | m | 26.5 | 2.20 | m |
| 25 | 123.2 | 5.04 | t (7.1) | 125.3 | 5.09 | t (7.1) | 142.0 | 6.60 | t (7.4) |
| 26 | 135.0 | / | | 138.7 | / | | 131.6 | / | |
| 27 | 36.5 | 2.15 m, 1.85 m | | 31.5 | 2.19 m, 2.00 m | | 23.6 | 2.34 m, 2.21 m | |
| 28 | 29.4 | 1.59 m, 1.16 m | | 27.4 | 1.46 | m | 28.3 | 1.43 overlap | |
| 29 | 77.0 | 3.02 | d (10.3) | 82.1 | 3.61 | dd (8.6, 4.1) | 81.8 | 3.59 | dd (8.1, 4.7) |
| 30 | 71.5 | / | | 79.4 | / | | 79.4 | / | |
| 31 | 26.1 | 1.02 | s | 25.7 | 1.14 | s | 25.7 | 1.13 | s |
| 32 | 24.4 | 0.96 | s | 22.7 | 0.98 | s | 22.7 | 0.97 | s |
| 33 | 15.9 | 1.53 | s | 57.9 | 3.91 | s | 168.2 | / | |
| 34 | 15.3 | 1.40 | s | 15.3 | 1.39 | s | 15.3 | 1.42 | s |
| 35 | 15.5 | 1.28 | s | 15.5 | 1.28 | s | 15.5 | 1.27 | s |
| 1′ | / | / | | 105.6 | / | | 105.7 | / | |
| 2′ | / | / | | 28.4 | 1.29 | s | 28.5 | 1.29 | s |
| 3′ | / | / | | 26.7 | 1.21 | s | 26.7 | 1.21 | s |
| 7—OH | / | 13.11 | s | / | 13.11 | s | / | 13.10 | s |
| 13—OH | / | 14.34 | s | / | 14.34 | s | / | 14.33 | s |

### (8) Compound C9

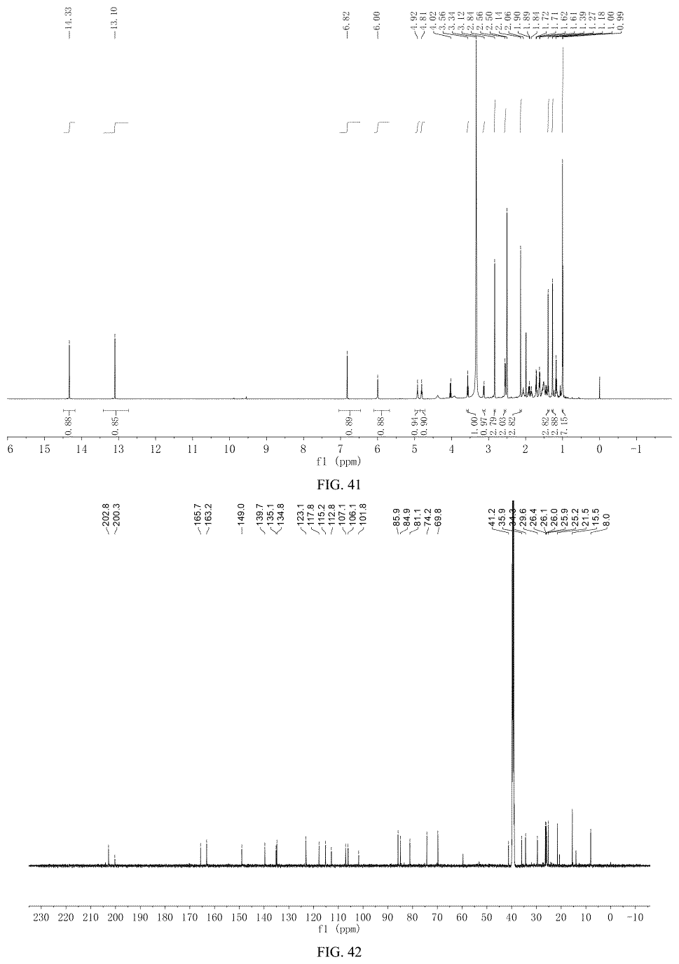

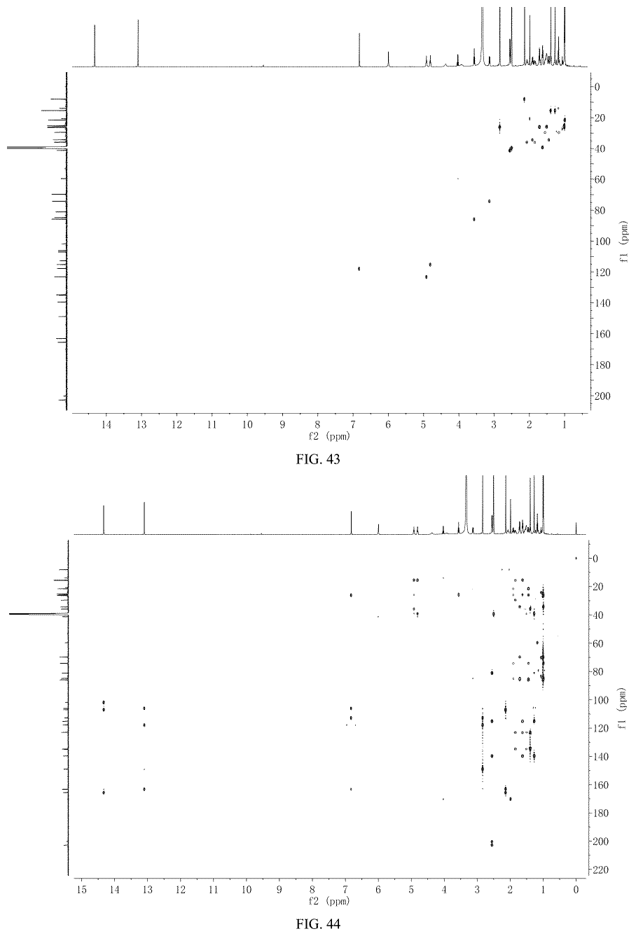

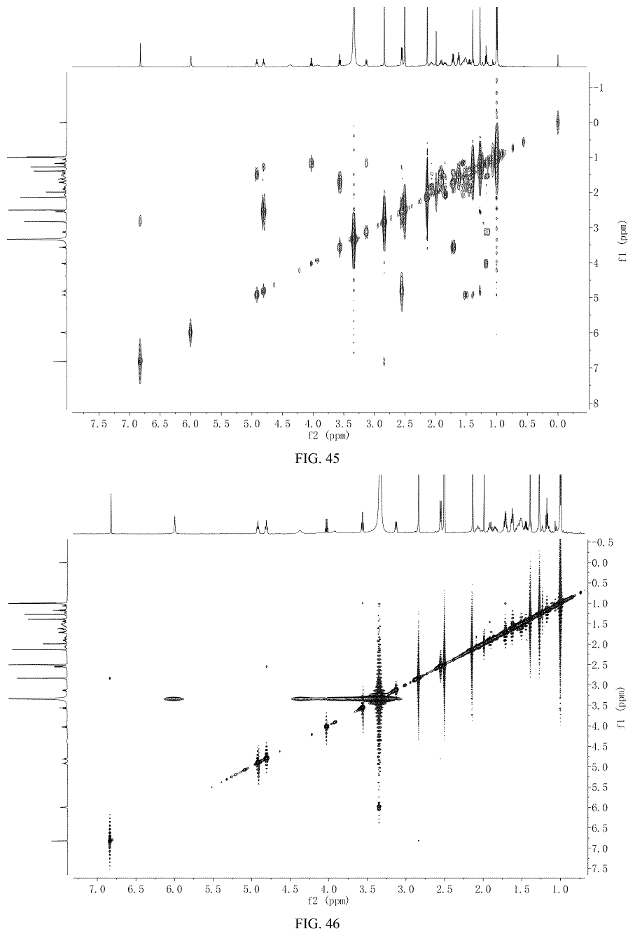

HRESIMS (negative ion) ion peak of compound C9: m/z 609.3343[M-H⁻]⁻, indicating that its molecular formula is C₃₅H₄₆O₉. According to the comprehensive analysis of ¹H NMR (FIG. 41), ¹³C NMR (FIG. 42), and HSQC spectrum (FIG. 43), it was presumed that the compound should contain 2 ketone carbonyl, 16 alkene carbons, and 17 sp³ hybridized carbons. The nuclear magnetic resonance signal of compound C9 is similar to the nuclear magnetic resonance signal of compound C1 in most parts, differing in C-24 to C-29 structural fragments. In the HMBC spectrum (FIG. 44), it could be seen that 61.00 (H-31) is associated with δ85.9 (C-29) and 69.8 (C-30), δ0.99 (H-33) is associated with δ74.5 (C-25), 84.9 (C-26), and 34.3 (C-27), and 63.13 (H-25) is associated with δ35.9 (C-23), 84.9 (C-26), and 34.3 (C-27). In the ¹H-¹H COSY spectrum (FIG. 45), it could be seen that 63.57 (H-29) is associated with δ1.71 (H-28), δ1.71 (H-28) is associated with δ1.91 and 1.45 (H-27), δ3.13 (H-25) is associated with δ1.56 and 1.19 (H-24), and 61.56, 1.19 (H-24) is associated with δ2.07, 1.85 (H-24). According to the evidence described above, the structures of C-24 to C-29 in compound C9 could be determined. In the NOESY spectrum (FIG. 46), it could be found that 60.99 (H-33) is associated with δ3.57 (H-29). The relative configurations of C-26 and C-29 substituents could be inferred. Finally, compound C9 was identified as having a structure shown in Table 6, and its nuclear magnetic resonance signal assignments are specifically shown in Table 4.

### (9) Compound C10

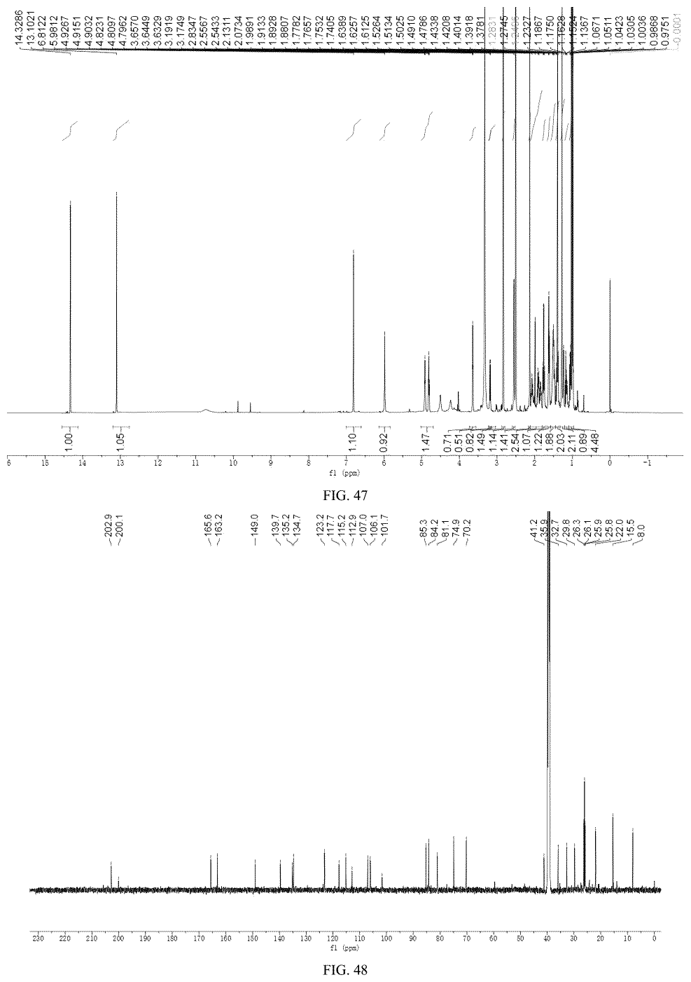

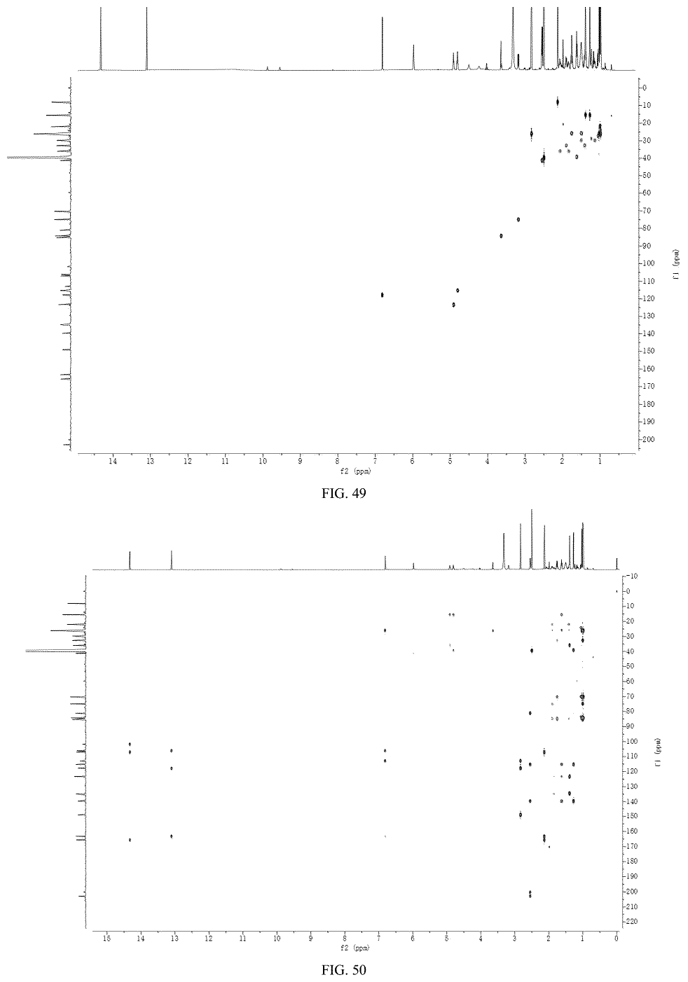

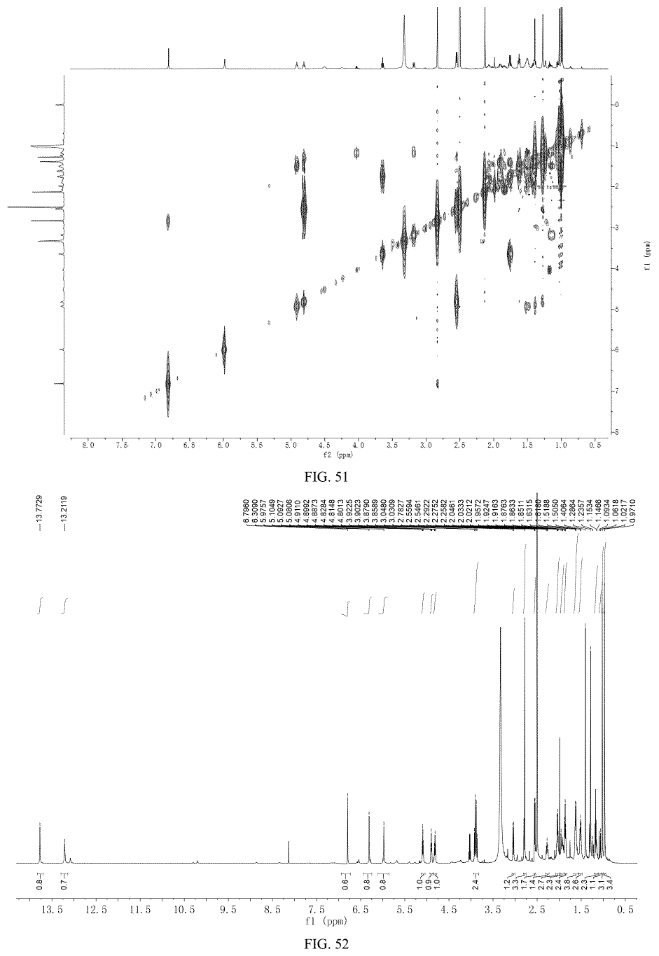

HRESIMS (negative ion) ion peak of compound C10: m/z 609.3343[M-H⁻]⁻, indicating that its molecular formula is C₃₅H₄₆O₉. The nuclear magnetic resonance signals of compound C10 and C9 are substantially the same, and it was inferred that compounds C10 and C9 had the same plane structure. According to the comprehensive analysis of ¹H NMR (FIG. 47), ¹³C NMR (FIG. 48), HSQC spectrum (FIG. 49), ¹H-¹H COSY (FIG. 50), and HMBC spectrum (FIG. 51) of compound C10, the above conclusion was demonstrated. The nuclear magnetic resonance signals of compounds C10 and C9 were carefully compared. Compounds C10 and C9 were slightly different in the structures of C-26 to C-29. The relative configurations of C-26 and C-29 substituents (different from compound C9) in compound C10 were inferred. Finally, compound C10 was identified as having a structure shown in Table 6, and its nuclear magnetic resonance signal assignments are specifically shown in Table 4.

### (10) Compound C11

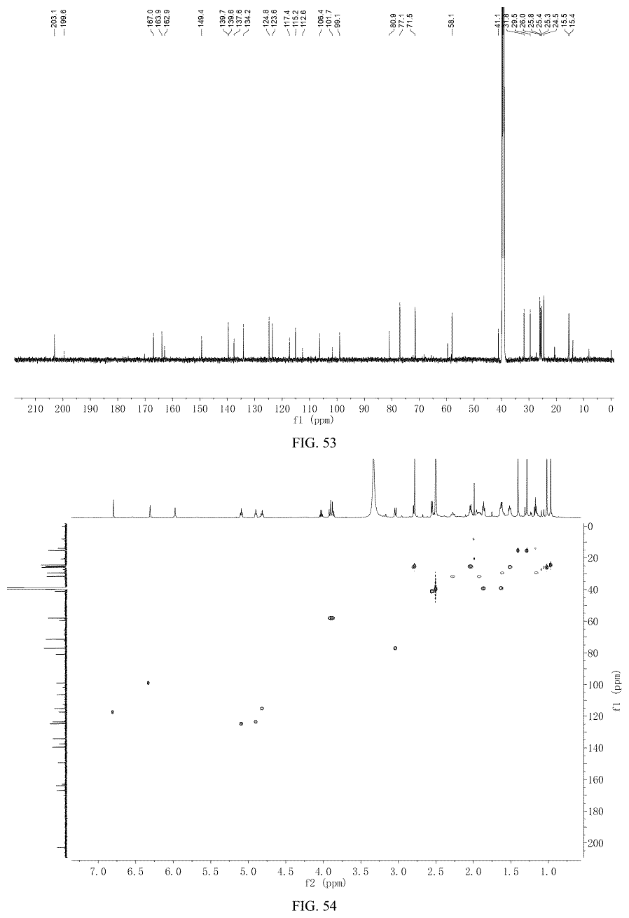

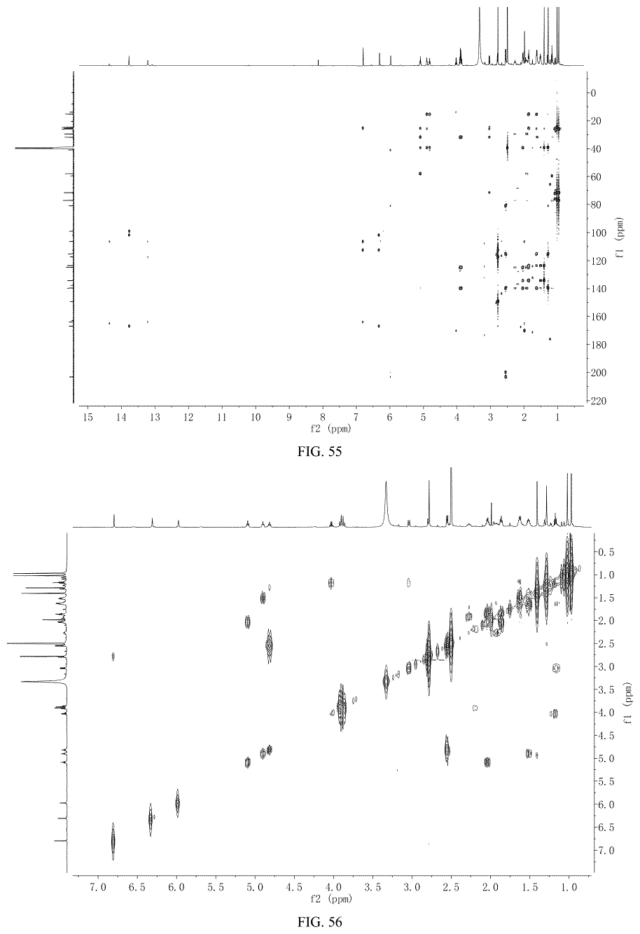

HRESIMS (negative ion) ion peak of compound C11: m/z 595.2946[M-H⁻]⁻, indicating that its molecular formula is C₃₄H₄₄O₉. According to the comprehensive analysis of ¹H NMR (FIG. 52), ¹³C NMR (FIG. 53), and HSQC spectrum (FIG. 54), it was presumed that the compound should contain 2 ketone carbonyl, 16 alkene carbons, and 16 sp³ hybridized carbons. The nuclear magnetic resonance signals of compounds C11 and C1 are highly similar.

After detailed comparison, the diterpene branched chains of compounds C11 and C1 have the same structure, and only small difference exists in the three-membered ring structure of the perinaphthenone. The hydrogen spectrum of compound C11 has one less methyl hydrogen signal at position C-14 (approximately at δ2.13) than the hydrogen spectrum of compound C1, but has one more aromatic hydrogen signal in the form of a single peak (δ6.31), indicating no methyl substitution at position C-12 in the compound C11 structure. In the HMBC spectrum (FIG. 55), δ13.78 (13-OH) is associated with δ101.7 (C-3), 99.1 (C-12), and 167.0 (C-13), and β 6.32 (H-12) is associated with δ101.7 (C-3) and 112.6 (C-10), further demonstrating the conclusion described above. Considering that compound C11 has the same biosynthetic pathway as that of compounds C1 and C2, the configurations of positions C-1 and C-29 in the structure of compound C11 are S and R configuration, respectively, and the configurations of 17(18)-ene, 21(22)-ene, and 25(26)-ene are E−, E−, and Z−, respectively. Finally, compound C11 was identified as having a structure shown in Table 6, and its nuclear magnetic resonance signal assignments are specifically shown in Table 4.

TABLE 4
Nuclear magnetic resonance data of compounds C9
to C11 (¹H NMR 600 MHz; ¹³C NMR 150 MHz; DMSO-d₆)

| | C9 | | | C10 | | | C11 | | |
|---|---|---|---|---|---|---|---|---|---|
| POS. | δ_{C} | δ_{H} (J, Hz) | | δ_{C} | δ_{H} (J, Hz) | | δ_{C} | δ_{H} (J, Hz) | |
| 1 | 81.1 | / | | 81.1 | / | | 80.9 | / | |
| 2 | 200.3 | / | | 200.1 | / | | 199.6 | / | |
| 3 | 101.8 | / | | 101.7 | / | | 101.7 | / | |
| 4 | 135.1 | / | | 135.2 | / | | 137.6 | / | |
| 5 | 106.1 | / | | 106.1 | / | | 106.4 | / | |
| 6 | 202.8 | / | | 202.9 | / | | 203.1 | / | |
| 7 | 163.2 | / | | 163.2 | / | | 162.9 | / | |
| 8 | 117.8 | 6.82 | s | 117.7 | 6.81 | s | 117.4 | 6.80 | s |
| 9 | 149.0 | / | | 149.0 | / | | 149.4 | / | |
| 10 | 112.8 | / | | 112.9 | / | | 112.6 | / | |
| 11 | 163.2 | / | | 163.2 | / | | 163.9 | / | |
| 12 | 107.1 | / | | 107.1 | / | | 99.1 | 6.31 | s |
| 13 | 165.7 | / | | 165.6 | / | | 167.0 | / | |
| 14 | 8.0 | 2.14 | s | 8.0 | 2.13 | s | / | / | |
| 15 | 26.1 | 2.83 | s | 26.1 | 2.83 | s | 25.3 | 2.78 | s |
| 16 | 41.2 | 2.55 | d (8.0) | 41.2 | 2.55 | d (8.0) | 41.1 | 2.55 | d (8.0) |
| 17 | 115.2 | 4.82 | t (7.9) | 115.2 | 4.80 | t (8.1) | 115.2 | 4.81 | t (8.1) |
| 18 | 139.7 | / | | 139.7 | / | | 139.6 | / | |
| 19 | 39.1 | 1.63 | m | 39.1 | 1.62 | m | 39.1 | 1.63 | m |
| 20 | 25.9 | 1.51 | m | 25.9 | 1.50 | m | 25.8 | 1.51 | m |
| 21 | 123.1 | 4.91 | t (6.8) | 123.2 | 4.90 | t (7.1) | 123.6 | 4.90 | t (7.3) |
| 22 | 134.8 | / | | 134.7 | / | | 134.2 | / | |
| 23 | 35.9 | 2.07 m, 1.85 m | | 35.9 | 2.07 m, 1.83 m | | 39.3 | 1.86 | m |
| 24 | 29.6 | 1.56 m, 1.16 m | | 29.8 | 1.50 m, 1.14 m | | 25.4 | 2.04 | m |
| 25 | 74.2 | 3.13 | d (9.9) | 74.9 | 3.18 | d (10.2) | 124.8 | 5.09 | t (7.1) |
| 26 | 84.9 | / | | 85.3 | / | | 139.7 | / | |
| 27 | 34.3 | 1.91 m, 1.45 m | | 32.7 | 1.90 m, 1.45 m | | 31.8 | 2.27 m, 1.92 m | |
| 28 | 26.0 | 1.71 | dd (7.6) | 25.8 | 1.76 | m | 29.5 | 1.62 m, 1.17 m | |
| 29 | 85.9 | 3.57 | t (7.5) | 84.2 | 3.64 | t (7.2) | 77.1 | 3.04 | dd (10.3) |
| 30 | 69.8 | / | | 70.2 | / | | 71.5 | / | |
| 31 | 26.4 | 1.00 | s | 26.3 | 0.99 | s | 26.0 | 1.02 | s |
| 32 | 25.2 | 1.00 | s | 26.1 | 1.03 | s | 24.5 | 0.97 | s |
| 33 | 21.5 | 0.99 | s | 22.0 | 1.00 | s | 58.1 | 3.89 | d (26.1, 12.1) |
| 34 | 15.5 | 1.39 | s | 15.5 | 1.39 | s | 15.4 | 1.41 | s |
| 35 | 15.5 | 1.28 | s | 15.5 | 1.27 | s | 15.5 | 1.29 | s |
| 7—OH | / | 13.10 | s | / | 13.10 | s | / | 13.21 | s |
| 13—OH | / | 14.33 | s | / | 14.33 | s | / | 13.78 | s |

### (11) Compound C12

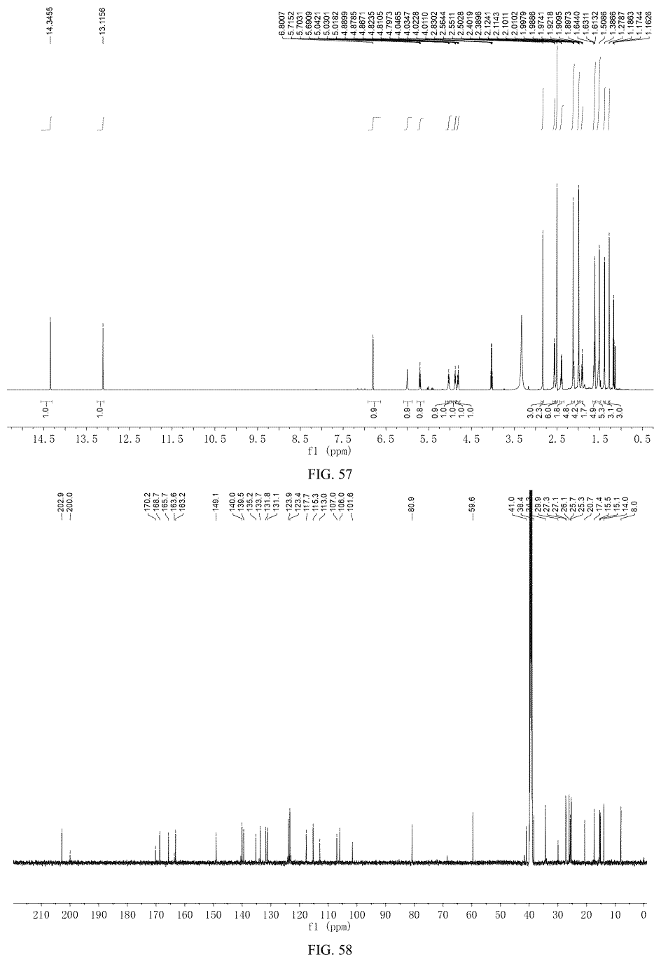

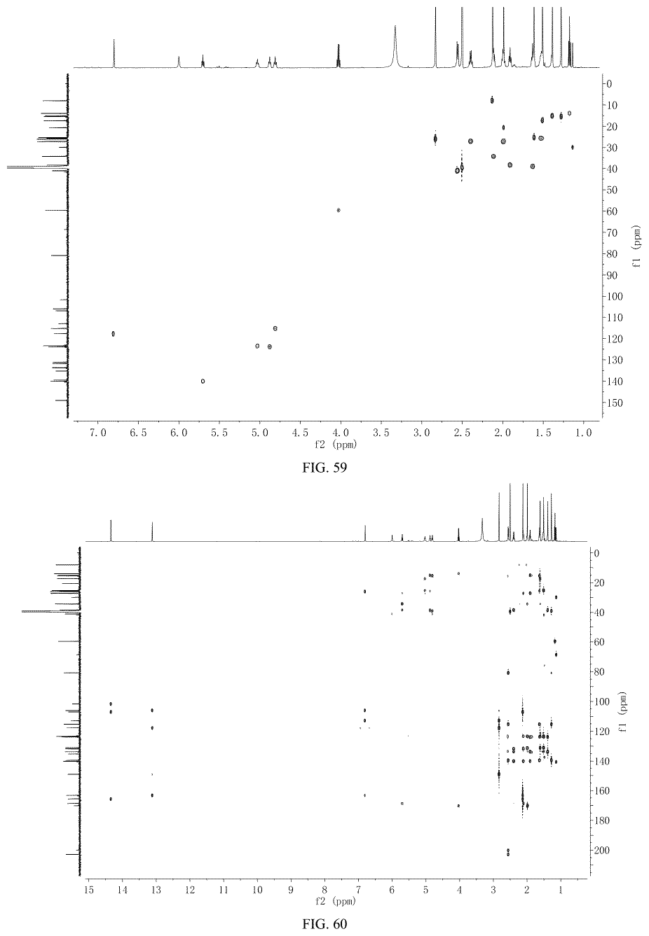

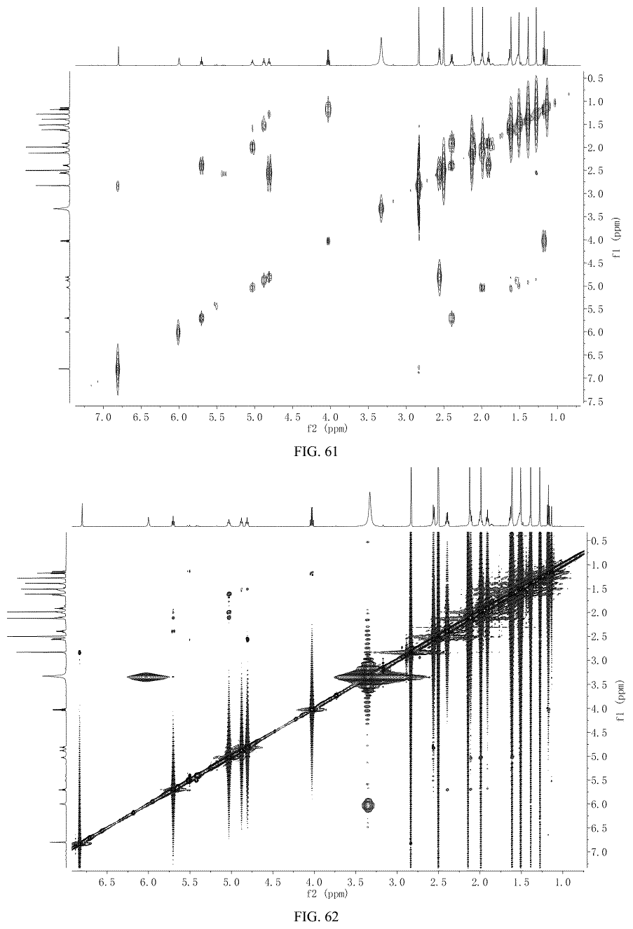

HRESIMS (negative ion) ion peak of compound C12: m/z 589.2827 [M-H⁻]⁻, indicating that its molecular formula is C₃₅H₄₂O₈. According to the comprehensive analysis of ¹H NMR (FIG. 57), ¹³C NMR (FIG. 58), and HSQC S spectrum (FIG. 59), it was presumed that the compound should contain 2 ketone carbonyl, 1 ester carbonyl, 18 alkene carbons, and 14 sp³ hybridized carbons. The nuclear magnetic resonance signals of compounds C12 and C2 are highly similar. After detailed comparison, it was found that the difference between C12 and C2 is only in the substituent at position C-26. In the HMBC spectrum (FIG. 60), δ5.70 (H-25) is associated with δ34.3 (C-27), 38.4 (C-23), and 168.7 (C-33), determining that position C-26 of compound C12 is substituted with one carboxyl (—COOH). In the NOESY spectrum (FIG. 62), δ5.70 (H-25) is associated with δ2.11 (H-27), demonstrating that the configuration of 25(26)-ene is E−. The configurations of other positions in the structure of compound C12 are the same as those of compound C2. Finally, compound C12 was identified as having a structure shown in Table 6, and its nuclear magnetic resonance signal assignments are specifically shown in Table 5.

### (12) Compound C13

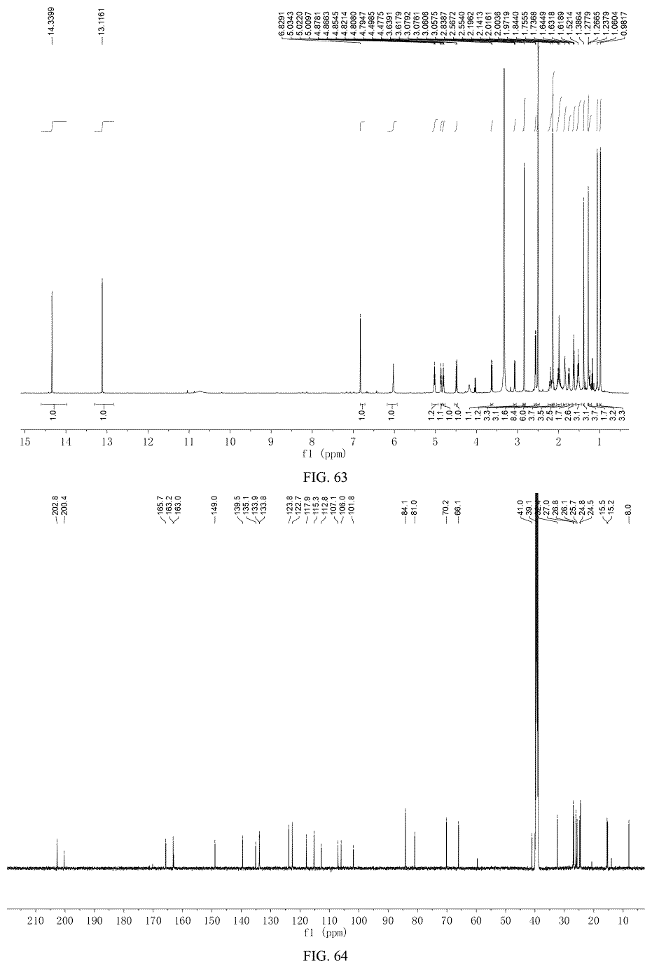

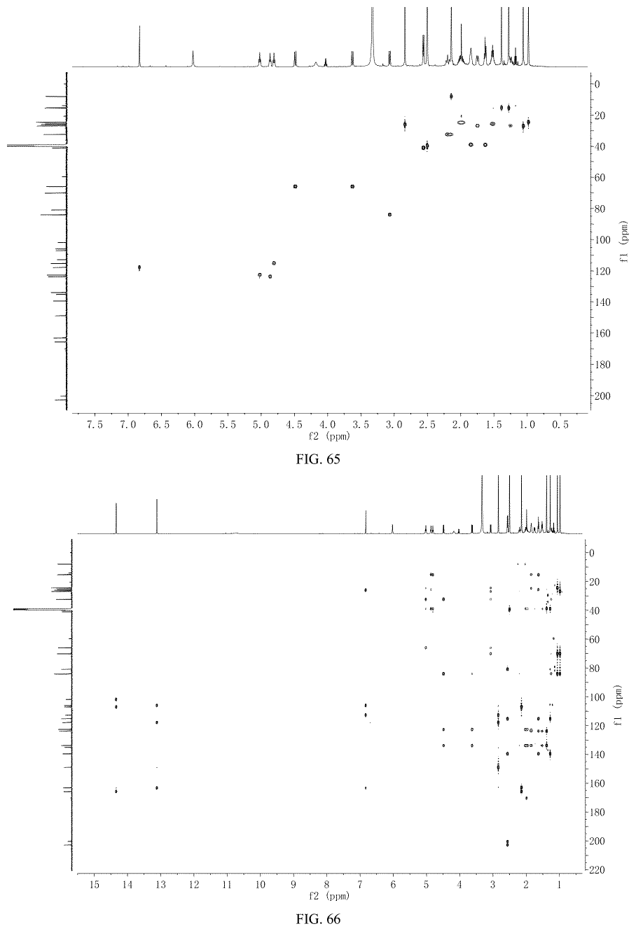

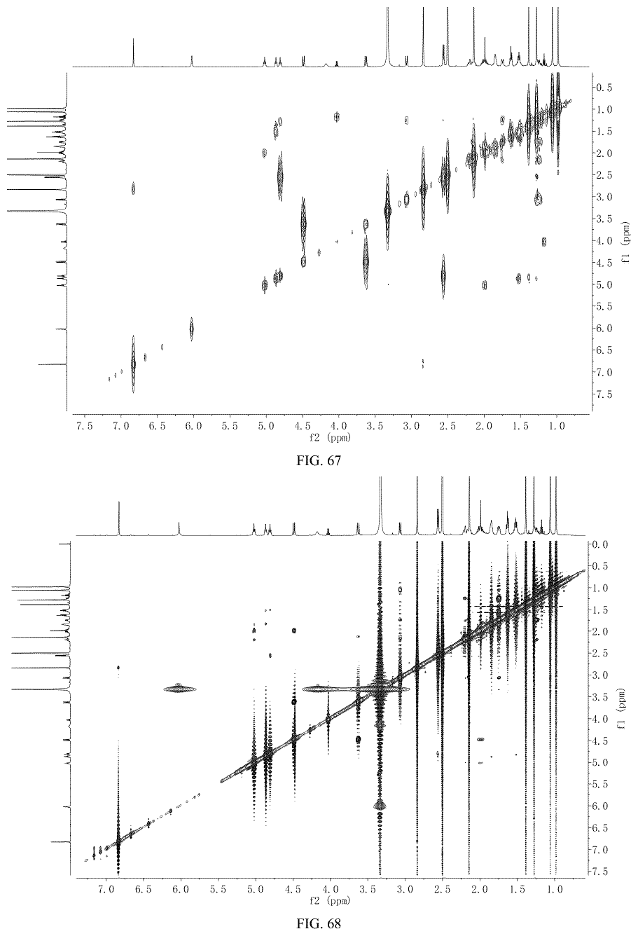

HRESIMS (negative ion) ion peak of compound C13: m/z 591.2969[M-H⁻]⁻, indicating that its molecular formula is C₃₅H₄₂O₇. According to the comprehensive analysis of ¹H NMR (FIG. 63), ¹³C NMR (FIG. 64), and HSQC spectrum (FIG. 65), it was presumed that the compound should contain 2 ketone carbonyl, 16 alkene carbons, and 17 sp³ hybridized carbons. The nuclear magnetic resonance signals of compounds C13 and C1 are similar. After detailed comparison, it was found that the difference between compounds C13 and C1 is in C-25 to C-29 structure fragments. In the HMBC spectrum (FIG. 66), H-33 (δ4.26, 4.06) is associated with C-25 (δ119.0) and C-26 (δ138.8), H-33 (δ4.49, 3.63) is associated with C-28 (δ84.1), and H-33 (δ3.06) is associated with C-28 (δ66.1), demonstrating that C-29 and C-30 were connected via an ether bond to form a six-membered ring structure. In the NOESY spectrum (FIG. 68), δ5.12 (H-25) is associated with δ2.50 (H-27), and δ4.49 (H-33) is associated with δ1.99 (H-24), demonstrating that the configuration of 25(26)-ene is Z−. The configurations of other positions in the structure are the same as those of compound C11. Finally, compound C13 was identified as having a structure shown in Table 6, and its nuclear magnetic resonance signal assignments are specifically shown in Table 5.

### (13) Compound C14

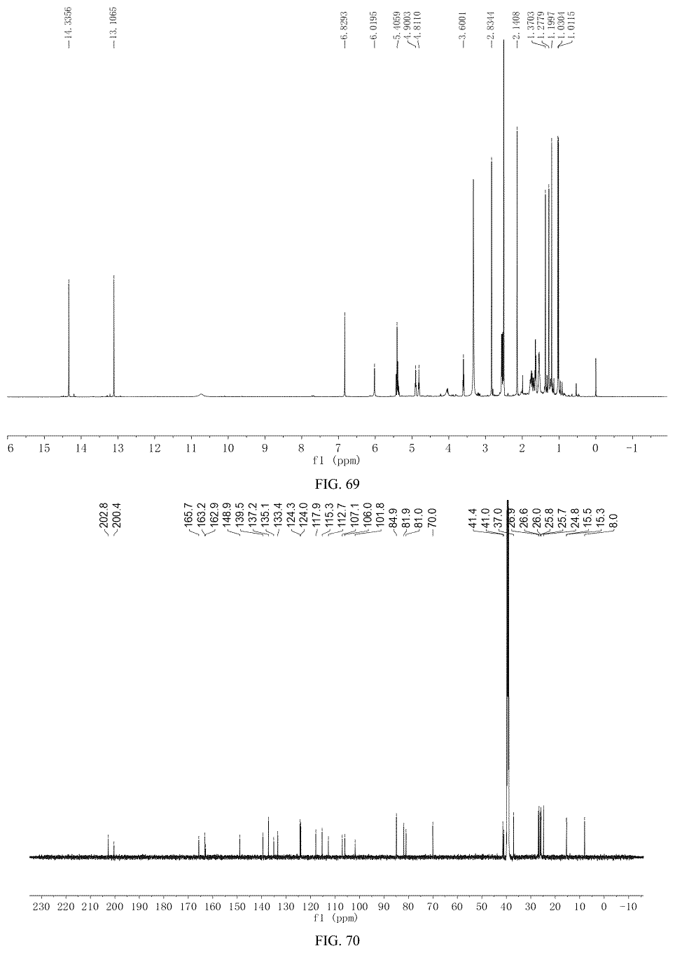

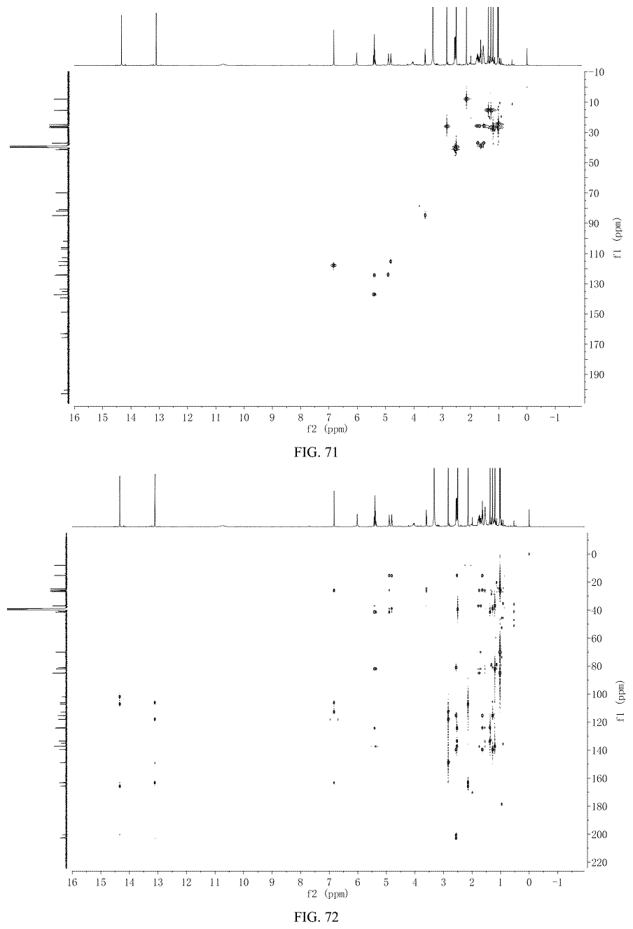

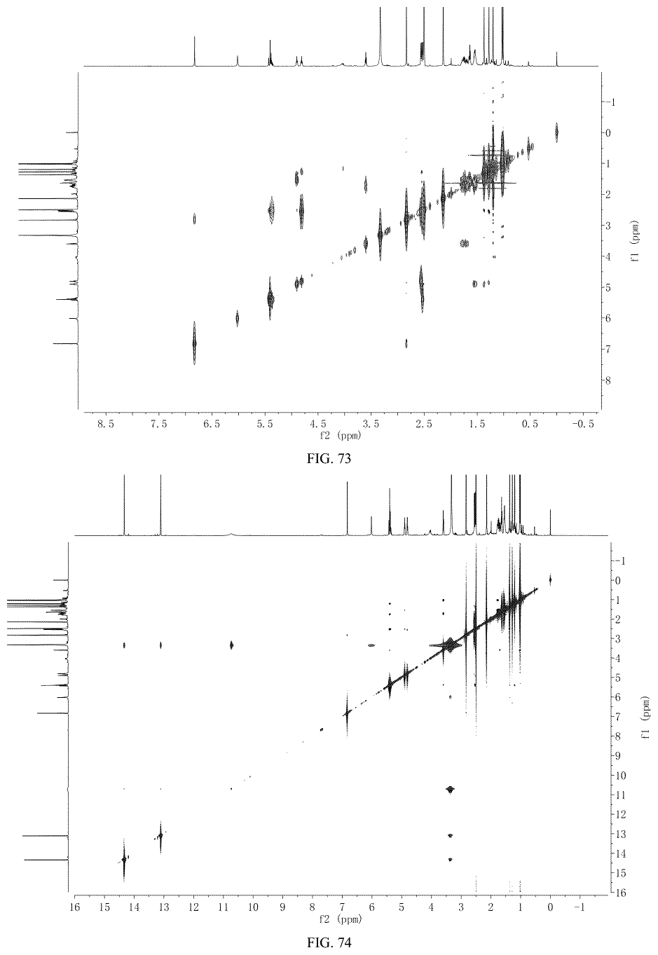

HRESIMS (negative ion) ion peak of compound C14: m/z 591.3248 [M-H⁻]⁻, indicating that its molecular formula is C₃₅1H₄₄O₈. According to the comprehensive analysis of ¹H NMR (FIG. 69), ¹³C NMR (FIG. 70), and HSQC spectrum (FIG. 71), it was presumed that the compound should contain 2 ketone carbonyl, 16 alkene carbons, and 17 sp³ hybridized carbons. The nuclear magnetic resonance signal of compound C14 is similar to the nuclear magnetic resonance signal of compound C1 in most parts, differing in C-24 to C-29 structural fragments. In the HMBC spectrum (FIG. 72), it could be seen that 61.03 (H-31) is associated with δ84.6 (C-29), and 70.0 (C-30), and δ1.20 (H-33) is associated with δ137.2 (C-25), δ1.9 (C-26), and 37.0 (C-27). In the ¹H-¹H COSY spectrum (FIG. 73), it could be seen that δ3.60 (H-29) is associated with δ1.78, 1.70 (H-28), δ1.78, 1.70 (H-28) is associated with δ1.76, 1.75 (H-27), and δ5.39 (H-24) is associated with δ5.41 (H-25) and δ2.53 (H-23), respectively. According to the evidence described above, the structures of C-24 to C-29 in compound C14 could be determined. In the NOESY spectrum (FIG. 74), it could be found that δ5.41 (H-25) is associated with δ2.53 (H-23), thus demonstrating that the configuration of 24(25)-ene is E−. Finally, compound C14 was identified as having a structure shown in Table 6, and its nuclear magnetic resonance signal assignments are specifically shown in Table 5.

TABLE 5
Nuclear magnetic resonance data of compounds C12
to C14 (¹H NMR 600 MHz; ¹³C NMR 150 MHz; DMSO-d₆)

| | C12 | | | C13 | | | C14 | | |
|---|---|---|---|---|---|---|---|---|---|
| POS. | δ_{C} | δ_{H} (J, Hz) | | δ_{C} | δ_{H} (J, Hz) | | δ_{C} | δ_{H} (J, Hz) | |
| 1 | 80.9 | / | | 81.0 | / | | 81.0 | / | |
| 2 | 200.0 | / | | 200.4 | / | | 200.4 | / | |
| 3 | 101.6 | / | | 101.8 | / | | 101.8 | / | |
| 4 | 135.2 | / | | 135.1 | / | | 135.1 | / | |
| 5 | 106.0 | / | | 106.0 | / | | 106.0 | / | |
| 6 | 202.9 | / | | 202.8 | / | | 202.8 | / | |
| 7 | 163.6 | / | | 163.0 | / | | 162.9 | / | |
| 8 | 117.7 | 6.80 | s | 117.9 | 6.83 | s | 117.9 | 6.83 | s |
| 9 | 149.1 | / | | 149.0 | / | | 148.9 | / | |
| 10 | 113.0 | / | | 112.8 | / | | 112.7 | / | |
| 11 | 163.2 | / | | 163.2 | / | | 163.2 | / | |
| 12 | 107.0 | / | | 107.1 | / | | 107.1 | / | |
| 13 | 165.7 | / | | 165.7 | / | | 165.7 | / | |
| 14 | 8.0 | 2.12 | s | 8.0 | 2.14 | s | 8.0 | 2.13 | s |
| 15 | 26.1 | 2.83 | s | 26.1 | 2.84 | s | 26.0 | 2.84 | s |
| 16 | 41.0 | 2.56 | d (8.0) | 41.0 | 2.56 | d (7.9) | 41.0 | 2.56 | d (7.9) |
| 17 | 115.3 | 4.81 | t (7.9) | 115.3 | 4.81 | t (8.0) | 115.3 | 4.81 | t (7.8) |
| 18 | 139.5 | / | | 139.5 | / | | 139.5 | / | |
| 19 | 39.2 | 1.63 | t (7.7) | 39.1 | 1.63 | t (7.8) | 39.0 | 1.64 | m |
| 20 | 25.7 | 1.52 | m | 25.7 | 1.52 | m | 25.7 | 1.54 | m |
| 21 | 123.9 | 4.88 | t (6.8) | 123.8 | 4.86 | t (7.1) | 124.0 | 4.90 | t (6.9) |
| 22 | 133.7 | / | | 133.8 | / | | 133.4 | / | |
| 23 | 38.4 | 1.91 | t (7.4) | 39.1 | 1.85 | m | 41.4 | 2.53 | d (6.1) |
| 24 | 27.3 | 2.40 | dd (14.8, 7.4) | 24.8 | 1.99 | m | 124.3 | 5.39 overlap | |
| 25 | 140.0 | 5.70 | t (7.3) | 122.7 | 5.02 | t (7.4) | 137.2 | 5.41 overlap | |
| 26 | 131.1 | / | | 133.9 | / | | 81.9 | / | |
| 27 | 34.3 | 2.11 | t (7.9) | 32.4 | 2.20 m, 2.15 m | | 37.0 | 1.76 m, 1.55 m | |
| 28 | 27.1 | 1.99 | dd (13.0, 7.4) | 26.8 | 1.75 m, 1.25 m | | 25.8 | 1.78 m, 1.70 m | |
| 29 | 123.4 | 5.03 | t (7.2) | 84.1 | 3.06 | dd (11.2, 1.9) | 84.9 | 3.60 | (6.7) |
| 30 | 131.8 | / | | 70.2 | / | | 70.0 | / | |
| 31 | 25.3 | 1.61 | s | 27.0 | 1.06 | s | 26.6 | 1.03 | s |
| 32 | 17.4 | 1.51 | s | 24.5 | 0.98 | s | 24.8 | 1.01 | s |
| 33 | 168.7 | / | | 66.1 | 4.49 d (12.6), 3.63 d (12.7) | | 26.9 | 1.20 | s |
| 34 | 15.1 | 1.39 | s | 15.2 | 1.39 | s | 15.3 | 1.37 | s |
| 35 | 15.5 | 1.28 | s | 15.5 | 1.28 | s | 15.5 | 1.28 | s |
| 7—OH | / | 13.12 | s | / | 13.12 | s | / | 13.11 | s |
| 13—OH | / | 14.35 | s | / | 14.34 | s | / | 14.34 | s |

TABLE 6
Structures of compounds C1 to C14

| Compound | | Molecular formula |
|---|---|---|
| No. | Structure of compound | Molecular weight |
| C1 | 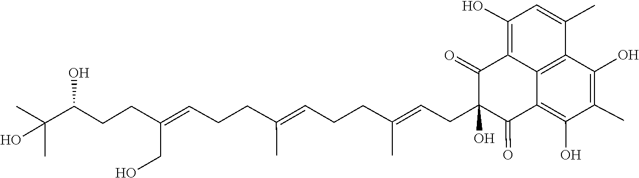 [SMILES: C/C(=C\CC/C(C)=C/C[C@]1(O)C(=O)c2c(O)cc(C)c3c(O)c(C)c(O)c(c23)C1=O)CC/C=C(\CO)CC[C@@H](O)C(C)(C)O] | C₃₅H₄₆O₉ 610 |
| C2 | 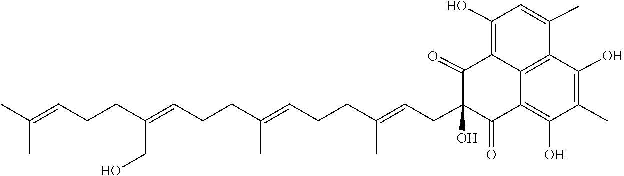 [SMILES: CC(C)=CCC/C(=C/CC/C(C)=C/CC/C(C)=C/C[C@]1(O)C(=O)c2c(O)cc(C)c3c(O)c(C)c(O)c(c23)C1=O)CO] | C₃₅H₄₄O₇ 576 |
| C3 | 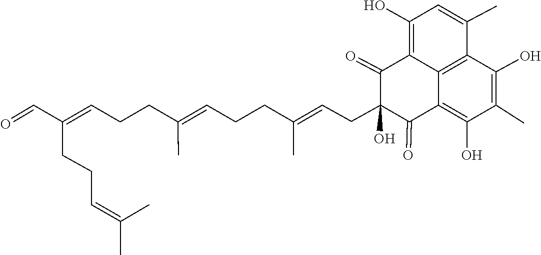 [SMILES: CC(C)=CCC/C(C=O)=C\CC/C(C)=C/CC/C(C)=C/C[C@]1(O)C(=O)c2c(O)cc(C)c3c(O)c(C)c(O)c(c23)C1=O] | C₃₅H₄₂O₇ 574 |
| C4 | 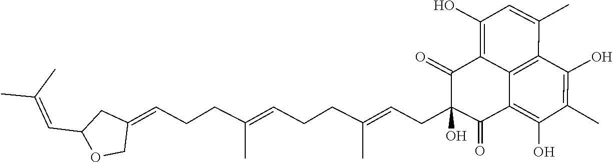 [SMILES: CC(C)=CC1C/C(=C/CC/C(C)=C/CC/C(C)=C/C[C@]2(O)C(=O)c3c(O)cc(C)c4c(O)c(C)c(O)c(c34)C2=O)CO1] | C₃₅H₄₂O₇ 574 |
| C5 | 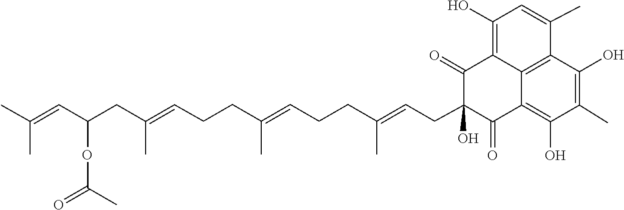 [SMILES: CC(=O)OC(C=C(C)C)C/C(C)=C/CC/C(C)=C/CC/C(C)=C/C[C@]1(O)C(=O)c2c(O)cc(C)c3c(O)c(C)c(O)c(c23)C1=O] | C₃₇H₄₆O₈ 618 |
| C6 | 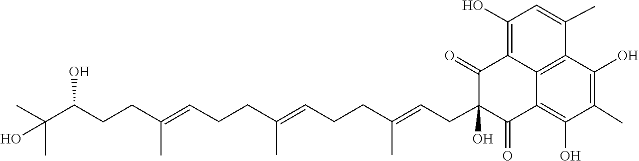 [SMILES: C/C(=C\CC/C(C)=C/C[C@]1(O)C(=O)c2c(O)cc(C)c3c(O)c(C)c(O)c(c23)C1=O)CC/C=C(\C)CC[C@@H](O)C(C)(C)O] | C₃₅H₄₆O₈ 594 |
| C7 | 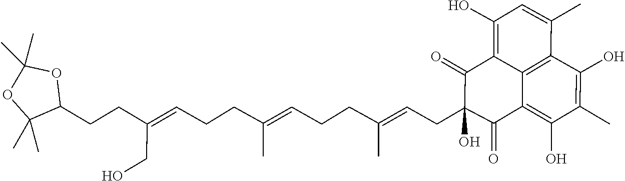 | C₃₈H₅₀O₉ 650 |
| C8 | 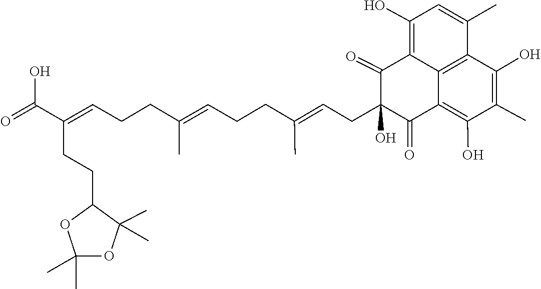 [SMILES: C/C(=C\CC/C(C)=C/C[C@]1(O)C(=O)c2c(O)cc(C)c3c(O)c(C)c(O)c(c23)C1=O)CC/C=C(\CCC1OC(C)(C)OC1(C)C)C(=O)O] | C₃₈H₄₈O₁₀ 664 |
| C9 | 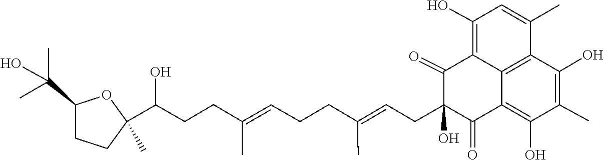 [SMILES: C/C(=C\C[C@]1(O)C(=O)c2c(O)cc(C)c3c(O)c(C)c(O)c(c23)C1=O)CC/C=C(\C)CCC(O)[C@@]1(C)CC[C@@H](C(C)(C)O)O1] | C₃₅H₄₆O₉ 610 |
| C10 | 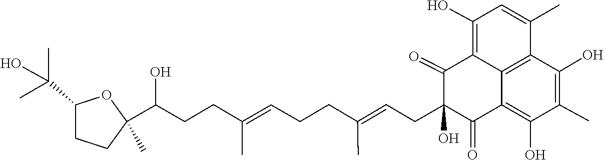 [SMILES: C/C(=C\C[C@]1(O)C(=O)c2c(O)cc(C)c3c(O)c(C)c(O)c(c23)C1=O)CC/C=C(\C)CCC(O)[C@@]1(C)CC[C@H](C(C)(C)O)O1] | C₃₅H₄₆O₉ 610 |
| C11 | 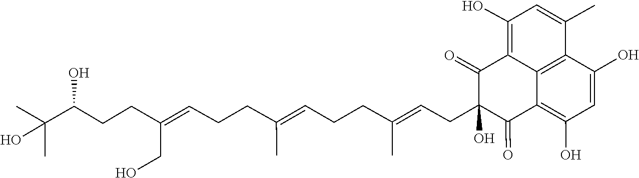 [SMILES: C/C(=C\CC/C(C)=C/C[C@]1(O)C(=O)c2c(O)cc(C)c3c(O)cc(O)c(c23)C1=O)CC/C=C(\CO)CC[C@@H](O)C(C)(C)O] | C₃₄H₄₄O₉ 596 |
| C12 | 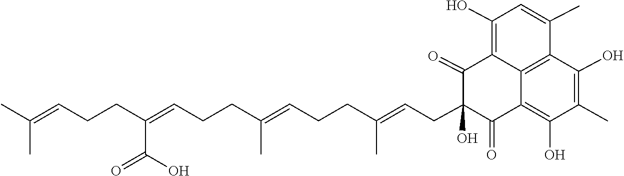 [SMILES: CC(C)=CCC/C(=C/CC/C(C)=C/CC/C(C)=C/C[C@]1(O)C(=O)c2c(O)cc(C)c3c(O)c(C)c(O)c(c23)C1=O)C(=O)O] | C₃₅H₄₂O₈ 590 |
| C13 | 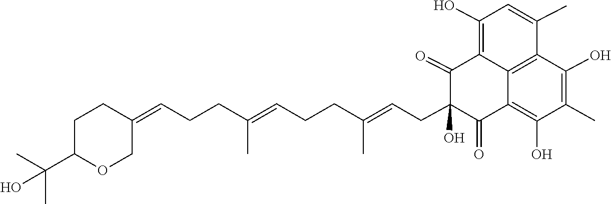 [SMILES: C/C(=C\CC/C(C)=C/C[C@]1(O)C(=O)c2c(O)cc(C)c3c(O)c(C)c(O)c(c23)C1=O)CC/C=C1/CCC(C(C)(C)O)OC1] | C₃₅H₄₄O₈ 592 |
| C14 | 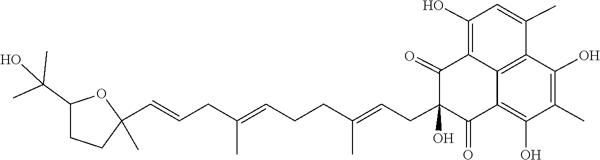 [SMILES: C/C(=C\CC/C(C)=C/C[C@]1(O)C(=O)c2c(O)cc(C)c3c(O)c(C)c(O)c(c23)C1=O)C/C=C/C1(C)CCC(C(C)(C)O)O1] | C₃₅H₄₄O₈ 592 |

### Example 5: Effect of Compounds C1 to C14 on PA Protein

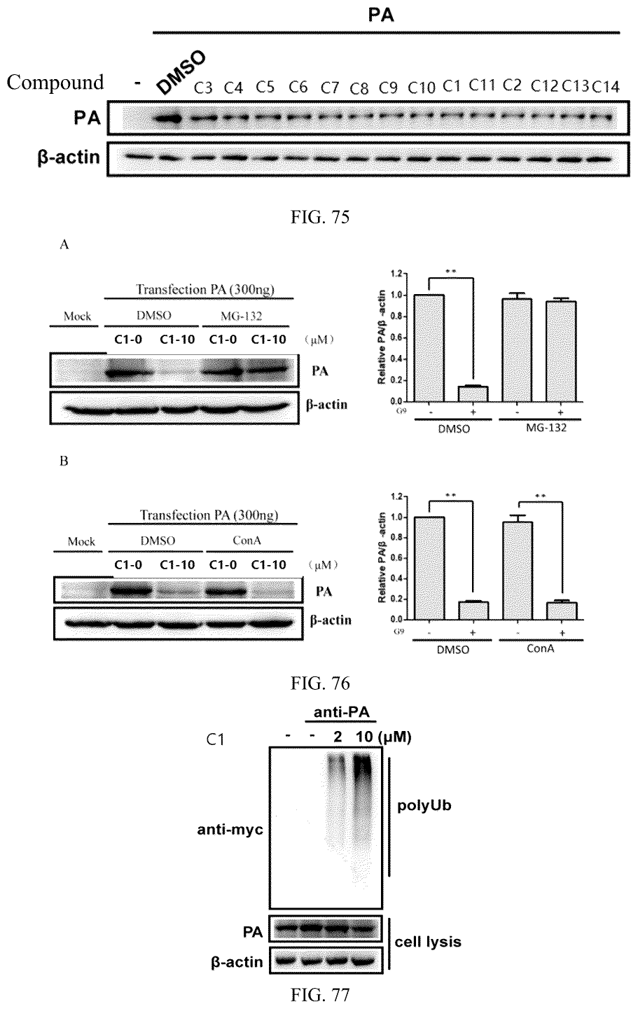

An HEK 293T cell suspension at 2.5×10⁵ cells/mL was inoculated in a 6-well plate at 2 mL per well. When the cells grew to 80%, the HEK 293T cell group was transfected with 500 ng of pHW2000-PA plasmid per well, and the culture medium was changed to DMEM culture medium containing 10% fetal bovine serum (FBS) 4 h after transfection. One group was added with 2 μL of 5.00 mM of each test compound per well, and the other group was then cultured for 24 h with DMSO (dimethyl sulfoxide) as a negative control. The culture medium was discarded. 80 μL of RIPA lysis buffer was added to each well, the lysate was transferred to a 1.5 mL EP tube and lysed on ice for 20 min. 20 μL of 5× protein loading buffer was added to each tube, and the tube was incubated in a metal bath at 100° C. for 30 min. The expression level of PA protein was detected by Western Blot. The detection results are shown in FIG. 75.

In addition, HEK293T cells were transfected with pHW2000-PA-Luc plasmid and treated with different concentrations of the compound. After 24 h, the expression level of Luc protein was measured, and the EC₅₀ value for the compound to degrade PA protein was calculated (Table 7).

TABLE 7
EC₅₀ results for compound to degrade influenza virus PA protein

| Compound | PA remaining EC₅₀ (μM) |
|---|---|
| C1 | 0.44 ± 0.09 |
| C2 | 0.65 ± 0.12 |
| C3 | 1.14 ± 0.26 |
| C4 | 1.15 ± 0.17 |
| C5 | 0.73 ± 0.23 |
| C6 | 2.09 ± 0.52 |
| C7 | 1.05 ± 0.19 |
| Ribavirin | — |
| C8 | 0.80 ± 0.12 |
| C9 | 1.04 ± 0.08 |
| C10 | 0.88 ± 0.08 |
| C11 | 0.62 ± 0.15 |
| C12 | 1.07 ± 0.10 |
| C13 | 0.71 ± 0.02 |
| C14 | 1.02 ± 0.03 |

### Example 6: Protease Inhibitor MG132 can Effectively Block Degradation of PA Protein by Compound C1

HEK239T cells were transfected with pHW183-PA plasmids and treated with different concentrations of compound C1 (2 μM and 10 μM), while adding lysosome inhibitor ConA (Concanavalin A) or proteasome inhibitor MG-132, and expression of PA protein was observed. The results showed that the expression level of PA protein was almost completely restored after treatment with the proteasome inhibitor MG-132 (FIG. 76A), but the restoration of PA protein after treatment with the lysosome inhibitor ConA was hardly affected (FIG. 76B), indicating that the degradation of PA protein by compound C1 is mainly accomplished through the proteasome pathway.

### Example 7: Compound C1 Promotes Ubiquitination Level of PA Protein

HEK293T cells were transfected with PA and myc-CW7 ubiquitin plasmids, and treated with different concentrations of compound C1 3.5 h after transfection. The cells were added with MG-132 and incubated 8 h before cell collection. The sample was collected 24 h after cell transfection, and captured by using a PA protein antibody. The expression level of ubiquitinated PA protein was detected by Western Blotting. The test results showed that compound C1 can promote the polyubiquitination level of PA protein (FIG. 77).

### Example 8: Discovery of E3 Ubiquitin Ligase TRIM25 by Using Surface Plasmon Resonance (SPR) Technology

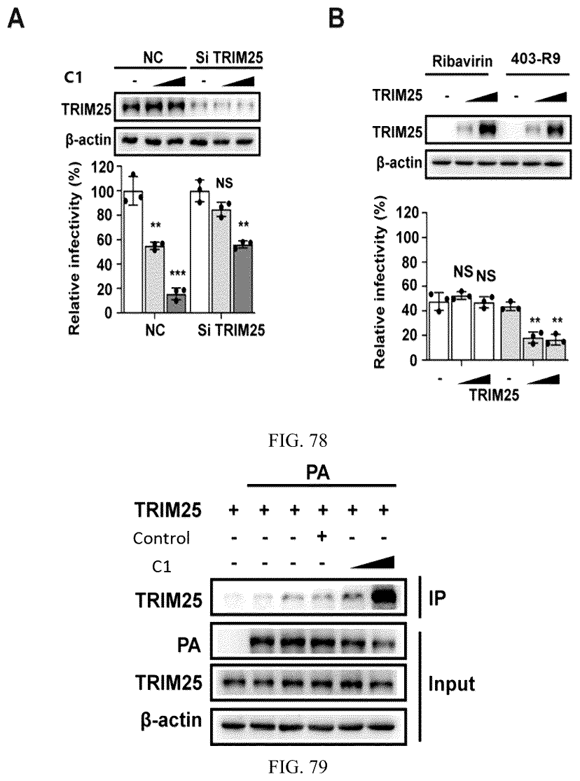

SPR technology is a classical method for detecting the binding of a small molecule to protein, and has the advantage of not requiring molecular labeling of the sample, i.e. not altering the properties of the small molecule, and being highly sensitive. The basic principle is that a small molecule is fixed on the surface of a chip, cell lysate continuously flows through the surface of the chip in the form of a solution, and change of the molecular concentration on the surface of the sensing chip during binding and dissociation process of the small molecule and the protein is recorded through LC-MS, so that the interaction between the small molecule and the protein is monitored in real time. The protein involved in polyubiquitination of PA protein was detected by using SPR technology. The test results showed that a total of seven host proteins involved in protein polyubiquitination were captured, including KEAP1, HERC5, RBP2, UBA7, TRIM25, ISG15, and UB2E2. Only after TRIM25 knockdown, the anti-influenza activity of compound C1 was significantly affected (FIG. 78A); and overexpression of TRIM25 in a TRIM25 knockout cell line could restore the anti-influenza activity of compound C1 to a normal cell level (FIG. 78B).

### Example 9: Compound C1 Promotes Interaction of TRIM25 with PA

HEK293T cells were transfected with PA and TRIM25 plasmids, and treated with different concentrations of compound C1 3.5 h after transfection. The cells were added with MG-132 and incubated 8 h before cell collection. The sample was collected 24 h after cell transfection, and captured by using a TRIM25 protein antibody. The expression level of the ubiquitinated PA protein was detected by Western Blotting. The test results showed that compound C1 can promote the interaction of TRIM25 with PA protein (FIG. 79).

### Example 10: Compounds C1-C14 Directly Bind to TRIM25 and PA Protein

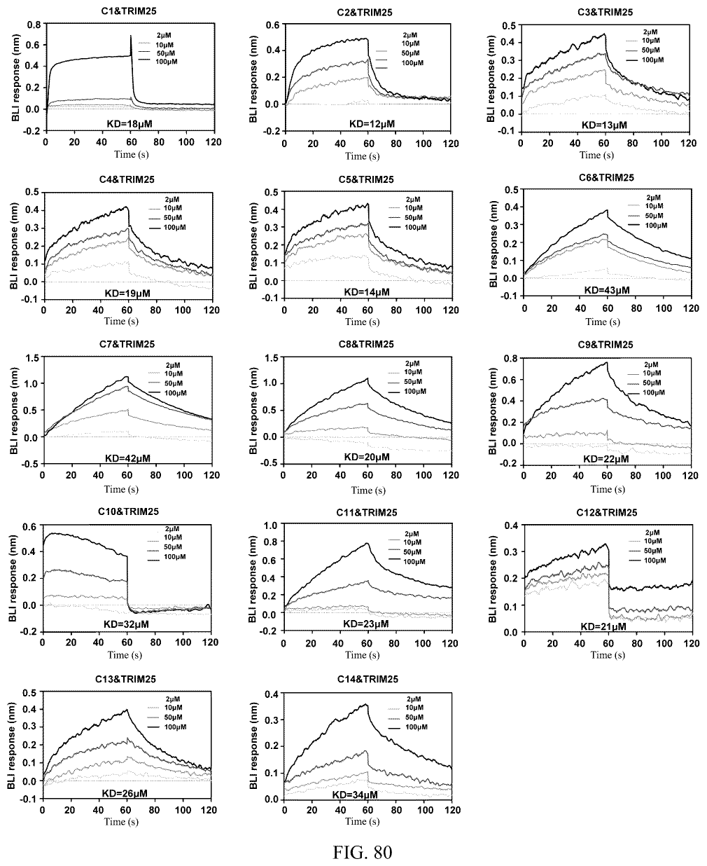

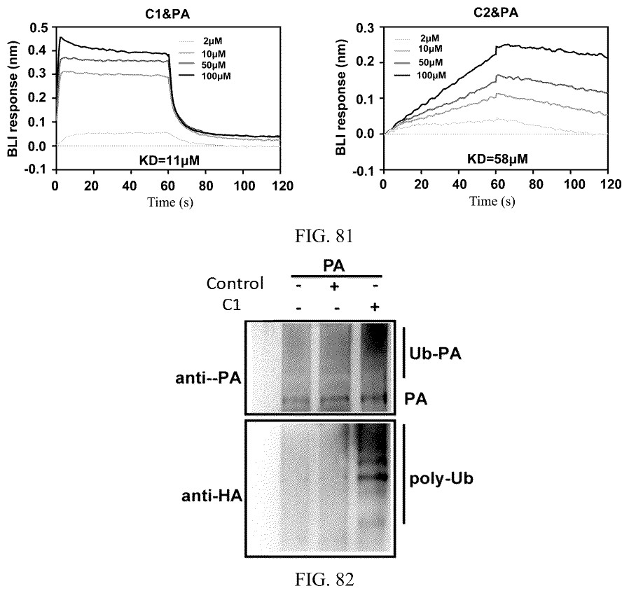

To further investigate the mechanism of degradation of PA protein by compound C1, the inventors tested the binding ability of the compound to TRIM25 protein and PA protein using biolayer interferometry (BLI). The test results showed that compounds C1-C14 can bind to TRIM25 protein, with KD values between 12 and 43 μM (FIG. 80). This result indicates that the binding ability of the compounds to TRIM25 is directly related to their function of inducing PA degradation. Meanwhile, the compounds also have the ability of binding PA in vitro. For example, compounds C1 and C2 could bind to PA protein, with KD values of 11 μM and 58 μM, respectively (FIG. 81). The above results suggest that this class of compounds can recruit TRIM25 to PA by binding to TRIM25 and PA, thereby inducing ubiquitination and degradation of PA.

### Example 11: Compound C1 Promotes Ubiquitination Level of PA Protein In Vitro

The in vitro ubiquitination test results showed that compound C1 can promote the polyubiquitination level of PA protein in vitro (FIG. 82).

### Example 12: Anti-Influenza Virus Activity

### (1) Cell Culturing

Human embryonic kidney epithelial cells 293T and 293T derived cell line 293T-Gluc were cultured in DMEM culture medium containing 10% fetal bovine serum (FBS).

### (2) Preparation of Recombinant Influenza a Virus

1.8×10⁶ 293T cells and 0.6×10⁶ MDCK cells were inoculated in a 3:1 ratio in a 10 cm cell culture dish. After 24 h of cultivation, 8 plasmids (pHW181-PB2, pHW182-PB1, pHW183-PA, pHW184-HA, pHW185-NP, pHW186-NA, pHW187-M, and pHW188-NS) of influenza A virus (IAV) A/WSN/33 (H1N1) were transfected in a transfection amount of 1.2 μg. The transfection reagent Lipofectamine2000 was used in an amount of 40 μL per dish according to the instruction. After 6 h of transfection, the culture medium was replaced by fresh DMEM culture medium. After 24 h of transfection, TPCK-trypsin with a final concentration of 1 μg/mL was added. After 48 h, the supernatant was collected, centrifuged at 1000 rpm for 5 min to remove cell debris, filtered through a 0.45 μm filter membrane, and aliquoted into small portions to give the A/WSN/33 (H1N1) recombinant influenza virus, which was stored in a freezer at −80° C.

The reverse genetics systems of the 8 plasmids of influenza A virus (IAV) were donated from Dr. Robert G. Webster, namely: pHW181-PB2, pHW182-PB1, pHW183-PA, pHW184-HA, pHW185-NP, pHW186-NA, pHW187-M, and pHW188-NS, respectively (Hoffmann, E., G. Neumann, et al. A DNA transfection system for generation of influenza A virus from eight plasmids[J]. Proc Natl Acad Sci USA, 2000, 97:6108-δ113).

### (3) EC₅₀ Assay Based on 293T-Gluc Cells

293T-Gluc cells (Gao Q, Wang Z, Liu Z, et al. A cell-based high-throughput approach to identify inhibitors of influenza A virus[J]. Acta Pharmaceutica Sinica B, 2014, 4(4): δ01-306) were plated in a 96-well plate, inoculated at 2.5×10⁴ cells per well, and incubated in 100 μL of DMEM culture medium containing 10% FBS. 24 h after cell plating, 1 μL of the gradiently diluted test compound was added per well (the test compound was dissolved in DMSO (dimethyl sulfoxide) and diluted with DMSO). The test compound was added, and 1 h later, virus infection was performed according to MOI 0.25. After 24 h, 10 μL of each supernatant was taken to detect the Gluc protein content for calculating the EC₅₀ (the concentration required to inhibit the virus by 50%). The experiment was repeated three times.

### Detection of Gaussia Luciferase Activity

250 μg of the substrate Coelenterazine-h lyophilized powder was dissolved in δ00 μL of absolute ethanol to give a 1.022 mM substrate mother liquor, which was stored at −20° C. Before measurement, the mother liquor was diluted in PBS at a ratio of 1:60 to give a substrate working solution. The working solution was left to stand at room temperature for 30 min for stabilization. Due to the instability of the substrate when exposed to light, it is necessary to avoid light treatment throughout the process. 10 μL of the cell culture supernatant (the cell supernatant after 24 h of culture after transfection in the Western Blot experiment described above) was put into a white opaque 96-well plate, the substrate working solution incubated in the dark was added to each well in an amount of 60 μL per well by using a Centro XS³ LB 960 autosampler, the signal was collected continuously for 0.5 s, and the measurement results were expressed in relative light units (RLU). Three sets of replicates were set up for the experiment. The experimental data were expressed as x̅±s, and plotted and statistically analyzed using GraphPad Prism 5.0.

### (4) Cell Viability Assay

CCK-8 (Cell Counting Kit-8) kit is a rapid and highly sensitive detection kit based on WST-8 (water-soluble tetrazolium salt, chemical name: 2-(2-methyloxy-4-nitrophenyl)-3-(4-nitrophenyl)-5-(2,4-disulfophenyl)-2H-tetrazole monosodium salt) and widely applied to cell proliferation and cytotoxicity. WST-8 is a compound similar to MTT, and can be reduced by some dehydrogenases in mitochondria to produce orange-yellow formazan in the presence of an electron coupling reagent. The more and faster the cell proliferation, the darker the color; and the greater the cytotoxicity, the lighter the color. For the same cells, there is a linear relationship between the depth of color and the number of cells. The number of living cells can be indirectly reflected by measuring the light absorption value via an enzyme-linked immunosorbent assay instrument at a wavelength of 450 nm. 293T-Gluc cells were inoculated in a 96-well plate at 2.5×10⁴ cells per well, and incubated in 100 μL of DMEM culture medium containing 10% FBS. 24 h after cell plating, 1 μL of the gradiently diluted test compound was added per well (the test compound was dissolved in DMSO and diluted with DMSO). Meanwhile, a blank control (only 100 μL of DMEM culture medium was added), a positive control (1 μL Ribavirin was added), and a negative control (1 μL DMSO was added) were set, and incubated at 37° C. for 48 h. The 96-well plate was taken out, and 10 μL of CCK-8 was added to each well. After 1-2 h of incubation at 37° C., the light absorption value of each well at a wavelength of 450 nm was detected by using an Enspire2300 multi-mode microplate reader to calculate the 50% cytotoxic concentration CC₅₀ (the drug concentration causing 50% cell death). The experiment was repeated three times.

TABLE 8
Results of anti-IAV activity of compound

| | Anti-influenza activity | |
|---|---|---|
| Compound | EC₅₀ (μM) | CC₅₀ (μM) |
| C1 | 0.45 ± 0.04 | >100 |
| C2 | 0.58 ± 0.06 | >100 |
| C3 | 1.42 ± 0.02 | >100 |
| C4 | 1.25 ± 0.01 | >100 |
| C5 | 0.57 ± 0.08 | >100 |
| C6 | 2.22 ± 0.04 | >100 |
| C7 | 0.78 ± 0.02 | >100 |
| Ribavirin | 27.85 ± 0.65 | >100 |
| C8 | 0.55 ± 0.03 | >100 |
| C9 | 1.02 ± 0.05 | >100 |
| C10 | 1.22 ± 0.01 | >100 |
| C11 | 0.91 ± 0.08 | >100 |
| C12 | 0.92 ± 0.02 | >100 |
| C13 | 0.65 ± 0.01 | >100 |
| C14 | 1.06 ± 0.09 | >100 |

As can be seen, compounds C1-C14 have good anti-influenza A virus activity to different extents, with EC₅₀ values against influenza virus between 0.45 and 2.22 μM. In addition, the compounds have CC₅₀ values of greater than 100 μM on 293T-Gluc cells. Therefore, the compounds have the characteristics of strong antiviral ability and low cytotoxicity.

The above description is only for the purpose of illustrating the preferred examples of the present invention, and is not intended to limit the scope of the present invention. Any modifications, equivalents, and the like made without departing from the spirit and principle of the present invention shall fall in the protection scope of the present invention.

The foregoing examples and methods described herein may vary based on the abilities, experience, and preferences of those skilled in the art.

The certain order in which the steps of the method are listed in the present invention does not constitute any limitation on the order of the steps of the method.

1. A compound, or a pharmaceutically acceptable salt, an ester, a stereoisomer, a prodrug, and a solvate thereof, wherein the compound has the following structure: 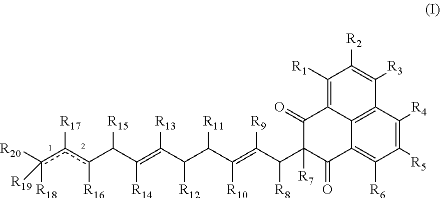
wherein,
the valence bond [symbol] at 1 and 2 represents a single or double bond, and 1 and 2 are not both double bonds;
R₁ to R₁₈ are independently selected from: H, alkyl, alkenyl, cycloalkyl, cycloalkylalkyl, aryl, aralkyl, heterocyclyl, heterocyclylalkyl, halogen, —CN, —NO₂, —COR^{A}, —C(O)OR^{A}, —OCOR^{A}, —C(O)NR^{A}R^{B}, —CH═NR^{A}, —OR^{A}, —OC(O)R^{A}, S(O)ₜ—R^{A}, —S(O)ₜ—NR^{A}R^{B}, —NR^{A}R^{B}, and —NR^{A}C(O)R^{B}; optionally, the H on each group may be substituted with one or more groups selected from: halogen, —CN, —CF₃, —NO₂, —CHO, —COOH, —C(O)NH₂, —OH, —OC(O)H, —SH, —S(O)₂H, and —NH₂;
R₁₉ and R₂₀ are independently selected from: H, alkyl, alkenyl, cycloalkyl, cycloalkylalkyl, aryl, aralkyl, heterocyclyl, and heterocyclylalkyl; optionally, the H on each group may be substituted with one or more groups selected from: substituted or unsubstituted heterocyclyl, halogen, —CN, —NO₂, —COR^{A}, —C(O)OR^{A}, —C(O)NR^{A}R^{B}, —CH═NR^{A}, —OR^{A}, —OC(O)R^{A}, —S(O)ₜ—R^{A}, —S(O)ₜ—NR^{A}R^{B}, —NR^{A}R^{B}, and —NR^{A}C(O)R^{B}; or, R₁₉ and R₂₀, together with the carbon atom to which they are attached, form substituted or unsubstituted cycloalkyl or heterocyclyl;
or, R₁₇ and R₁₉, together with the carbon atoms to which they are attached, form substituted or unsubstituted cycloalkyl or heterocyclyl;
t is selected from 0, 1, and 2;
each R^{A} and R^{B} are independently selected from: H, alkyl, cycloalkyl, alkenyl, aryl, heterocyclyl, and halogen.

2. The compound according to claim 1, wherein R₁ is selected from: —OH, —O(C1-10 alkyl), —SH, —S(C1-10 alkyl), —S(O)₂H, and —S(O)₂(C1-10 alkyl); preferably, R₁ is —OH;
R₂ is selected from: H, halogen, —CN, —CF₃, —NO₂, —CHO, —COOH, —C(O)NH₂, and —NH₂;
preferably, R₂ is H;
R₃ is selected from: C1-10 alkyl, C1-10 haloalkyl, C1-10 alkenyl, C3-6 cycloalkyl, and C4-10 cycloalkylalkyl, especially C1-10 alkyl; preferably, R₃ is methyl;
R₄ is selected from: —OH, —O(C1-10 alkyl), —SH, —S(C1-10 alkyl), —S(O)₂H, and —S(O)₂(C1-10 alkyl); preferably, R₄ is —OH;
R₅ is selected from: H, C1-10 alkyl, C1-10 haloalkyl, C1-10 alkenyl, C3-6 cycloalkyl, and C4-10 cycloalkylalkyl; preferably, R₅ is H or methyl;
R₆ is selected from: —OH, —O(C1-10 alkyl), —SH, —S(C1-10 alkyl), —S(O)₂H, and —S(O)₂(C1-10 alkyl); preferably, R₆ is —OH;
R₇ is selected from: —OH, —O(C1-10 alkyl), —SH, —S(C1-10 alkyl), —S(O)₂H, and —S(O)₂(C1-10 alkyl); preferably, R₇ is —OH.

3. The compound according to claim 1, wherein R₈, R₉, R₁₁, R₁₂, R₁₃, R₁₈, and R₁₆ are independently selected from: H, halogen, —CN, —CF₃, —NO₂, —CHO, —COOH, —C(O)NH₂, and —NH₂;
preferably, R₈, R₉, R₁₁, R₁₂, R₁₃, R₁₅, and R₁₆ are all H.

4. The compound according to claim 1, wherein R₁₀ and R₁₄ are independently selected from: C1-10 alkyl, C1-10 haloalkyl, C1-10 alkenyl, C3-6 cycloalkyl, and C4-10 cycloalkylalkyl; preferably, R₁₀ and R₁₄ are both methyl.

5. The compound according to claim 1, wherein
R₁₇ is selected from: H, —OH, —O(C1-10 alkyl), —SH, —S(C1-10 alkyl), —S(O)₂H, and —S(O)₂(C1-10 alkyl);
preferably, R₁₇ is H or —OH.

6. The compound according to claim 1, wherein R₁₈ is selected from: H, C1-10 alkyl, C1-10 haloalkyl, halogen, —CN, —CF₃, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl); optionally, one or more H on each group may be substituted with the following groups selected from: halogen, —CN, —CF₃, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl);
preferably, R₁₈ is selected from: H, C1-6 alkyl, C1-6 haloalkyl, C1-6 hydroxy-substituted alkyl, —CHO, —CO(C1-10 alkyl), —COOH, and —C(O)O(C1-10 alkyl);
more preferably, R₁₈ is selected from: H, methyl, ethyl, n-propyl, isopropyl, —CH₂OH, —COOH, —COOCH₃, and —CHO.

7. The compound according to claim 1, wherein
R₁₉ has the following structure: 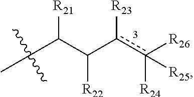
wherein, [symbol]
represents a single or double bond;
R₂₁ to R₂₄ are independently selected from: H, C1-10 alkyl, C1-10 haloalkyl (such as fluoroalkyl, e.g. trifluoromethyl), C1-10 alkenyl, C3-6 cycloalkyl, C4-10 cycloalkylalkyl, phenyl, four- to six-membered heterocycloalkyl, halogen, —CN, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl); or two of R₂₁ to R₂₄, together with the carbon atom(s) therebetween, form substituted or unsubstituted cycloalkyl or heterocyclyl; when [symbol] represents a double bond, R₂₄ is absent;
R₂₅ and R₂₆ are independently selected from: C1-10 alkyl, C1-10 haloalkyl, C1-10 alkenyl, C3-6 cycloalkyl, C4-10 cycloalkylalkyl, phenyl, and four- to six-membered heterocycloalkyl.

8. The compound according to claim 7, wherein R₂₅ and R₂₆ are independently selected from C1-10 alkyl; preferably, R₂₅ and R₂₆ are both methyl.

9. The compound according to claim 7, wherein R₂₁ is H, R₂₂ is selected from: H, —OC(O)H, and —OC(O)(C1-10 alkyl), R₂₃ is selected from: H, —OH, —O(C1-10 alkyl), —SH, and —S(C1-10 alkyl), and R₂₄ is selected from —OH and —O(C1-10 alkyl); or
R₂₀ and R₂₂, together with the carbon atoms therebetween, form heterocyclyl; or
R₂₀ and R₂₃, together with the carbon atoms therebetween, form heterocyclyl; or
R₂₃ and R₂₄, together with the carbon atoms therebetween, form heterocyclyl.

10. The compound according to claim 9, wherein the heterocyclyl is selected from: 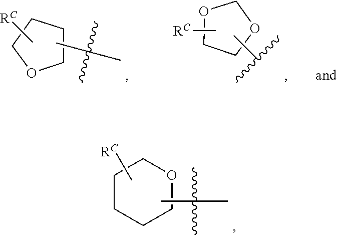
wherein R^{C} is one or more independent substituents on the ring and is selected from: C1-10 alkyl, C1-10 haloalkyl, C1-10 hydroxy-substituted alkyl, C1-10 alkenyl, halogen, —CN, —NO₂, —CHO, —CO(C1-10 alkyl), —COOH, —C(O)O(C1-10 alkyl), —C(O)NH₂, —C(O)N(C1-10 alkyl)(C1-10 alkyl), —OH, —O(C1-10 alkyl), —OC(O)H, —OC(O)(C1-10 alkyl), —SH, —S(C1-10 alkyl), —S(O)₂H, —S(O)₂(C1-10 alkyl), —NH₂, —N(C1-10 alkyl)(C1-10 alkyl), —NHC(O)H, and —N(C1-10 alkyl)C(O)(C1-10 alkyl).

11. The compound according to claim 1, wherein
R₁₉ is selected from: 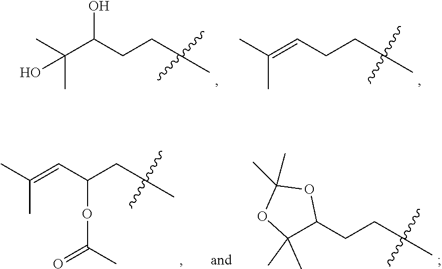
or
R₁₉ and R₂₀, together with the carbon atom to which they are attached, form a group selected from:

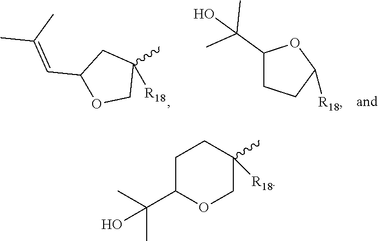

12. The compound according to claim 1, wherein the compound is selected from the following structures:

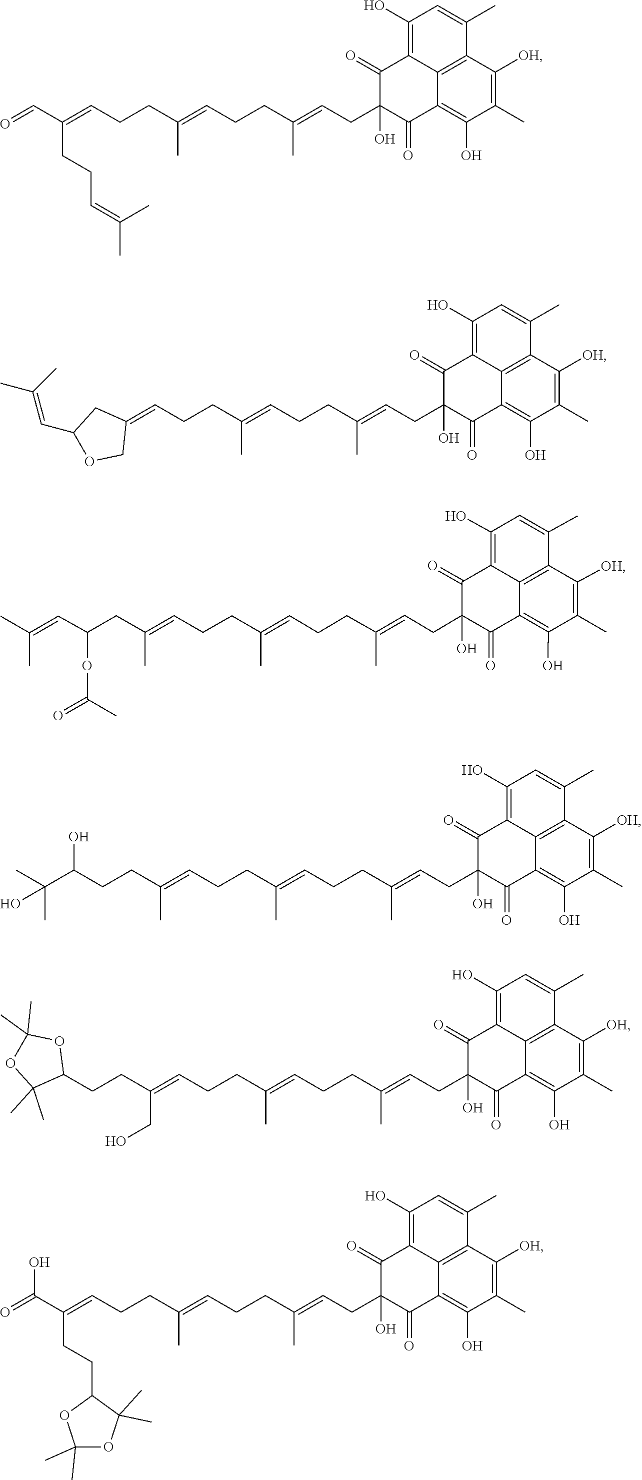

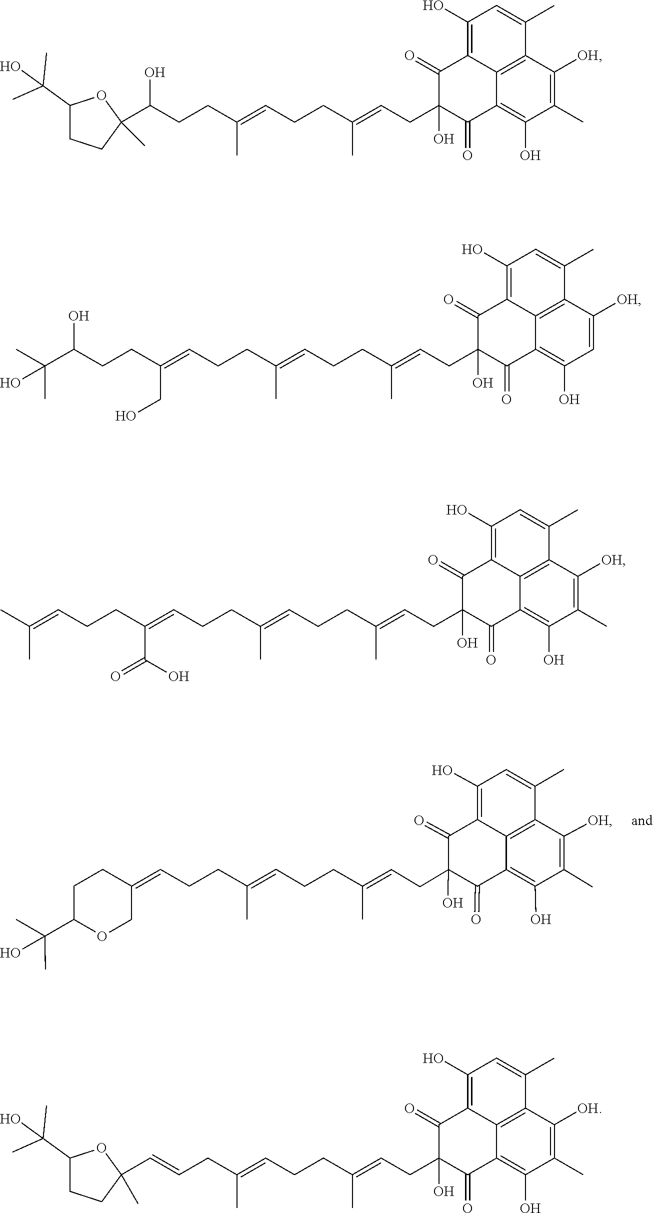

13. The compound according to claim 1, wherein the stereoisomer is selected from the following structures:

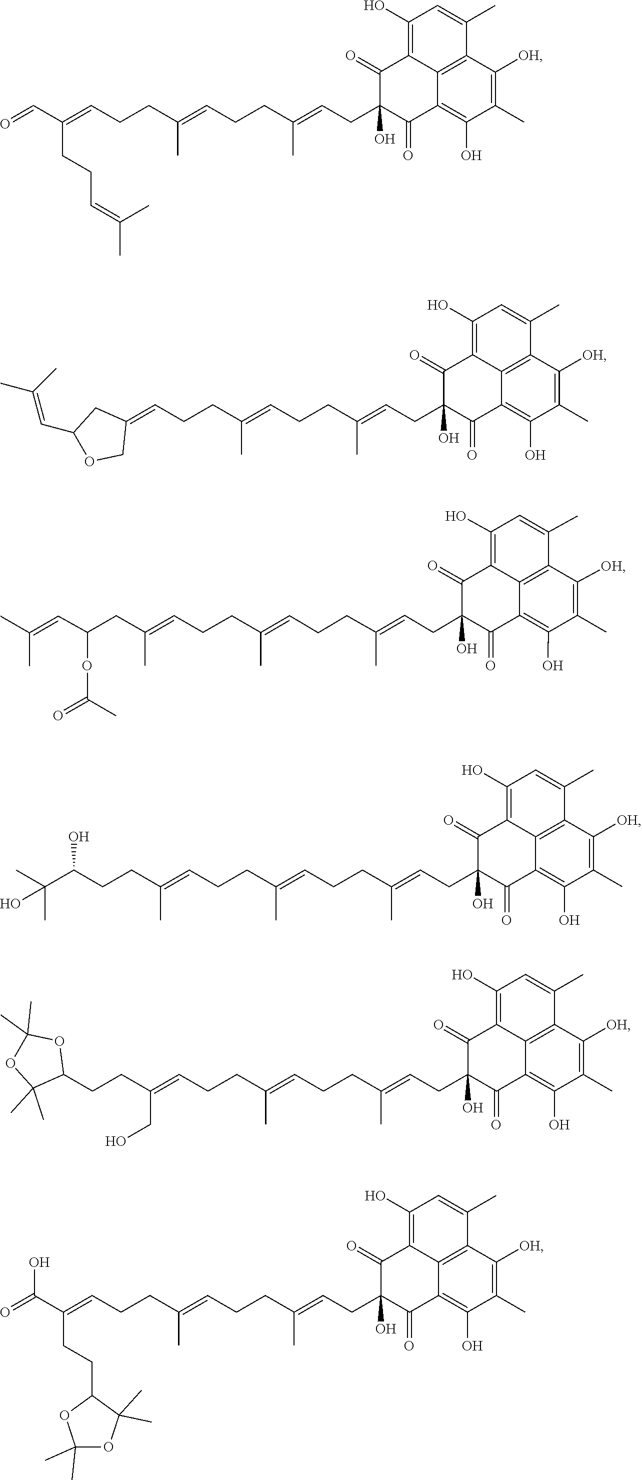

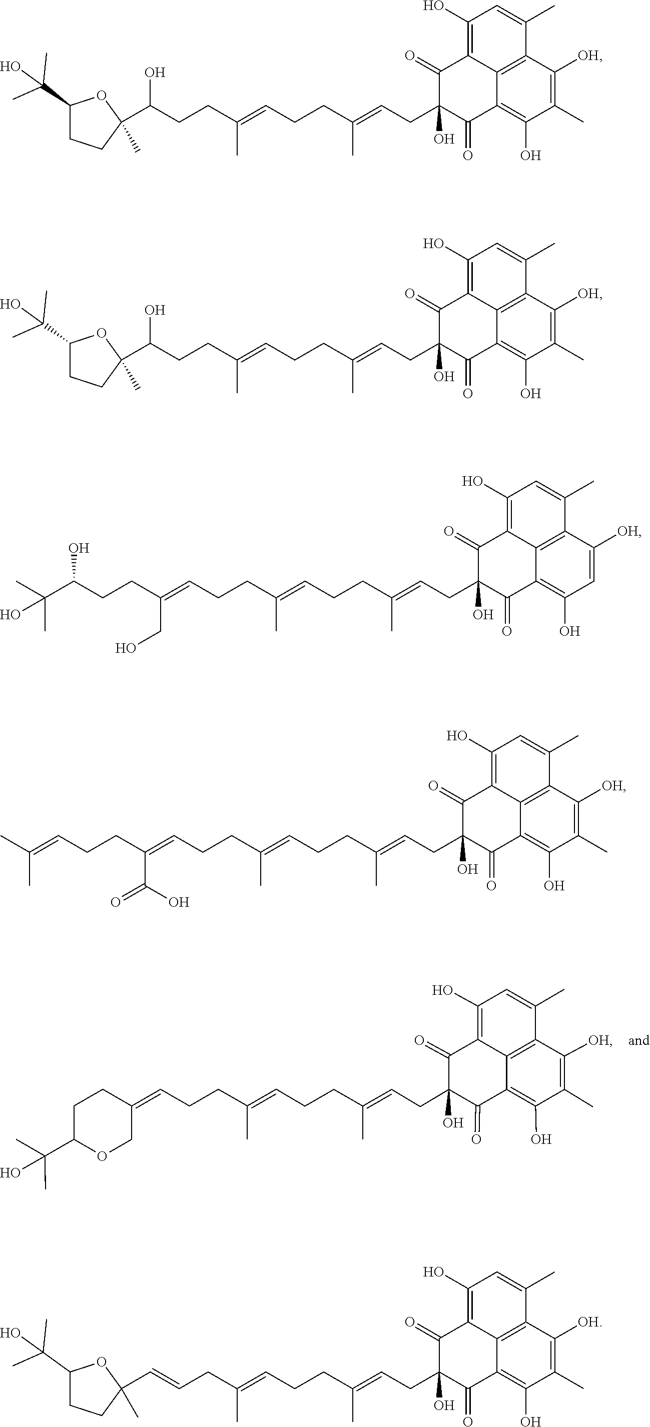

14. A pharmaceutical composition comprising the compound according to claim 1 or a pharmaceutically acceptable salt, an ester, a stereoisomer, a prodrug and a solvate thereof, and one or more pharmaceutically acceptable excipients.

15. A method for preventing and/or treating a disease, comprising a step of administering to a subject in need thereof an effective amount of the compound according to claim 1 or a pharmaceutically acceptable salt, an ester, a stereoisomer, a prodrug and a solvate thereof.

16. The method according to claim 15, wherein the disease is a disease caused by infection of a pathogen or a tumor;
preferably, the disease caused by infection of a virus is selected from: influenza, SARS, COVID-19, viral hepatitis, AIDS, rabies, Dengue fever, and Ebola virus disease;
preferably, the tumor is selected from: breast cancer, prostate cancer, ovarian cancer, cervical cancer, skin cancer, pancreatic cancer, colorectal cancer, kidney cancer, liver cancer, brain cancer, lymphoma, leukemia, lung cancer, melanoma, stomach cancer, gastroesophageal adenocarcinoma, esophageal cancer, small intestine cancer, cardiac cancer, bladder cancer, anal cancer, gallbladder cancer, bile duct cancer, teratoma, and heart tumor.

17. Use of the compound according to claim 1 or a pharmaceutically acceptable salt, an ester, a stereoisomer, a prodrug and a solvate thereof as a ligand of E3 ubiquitin ligase TRIM25 or in preparation of a proteolysis targeting chimera PROTAC;
preferably, the compound is selected from the following structures: 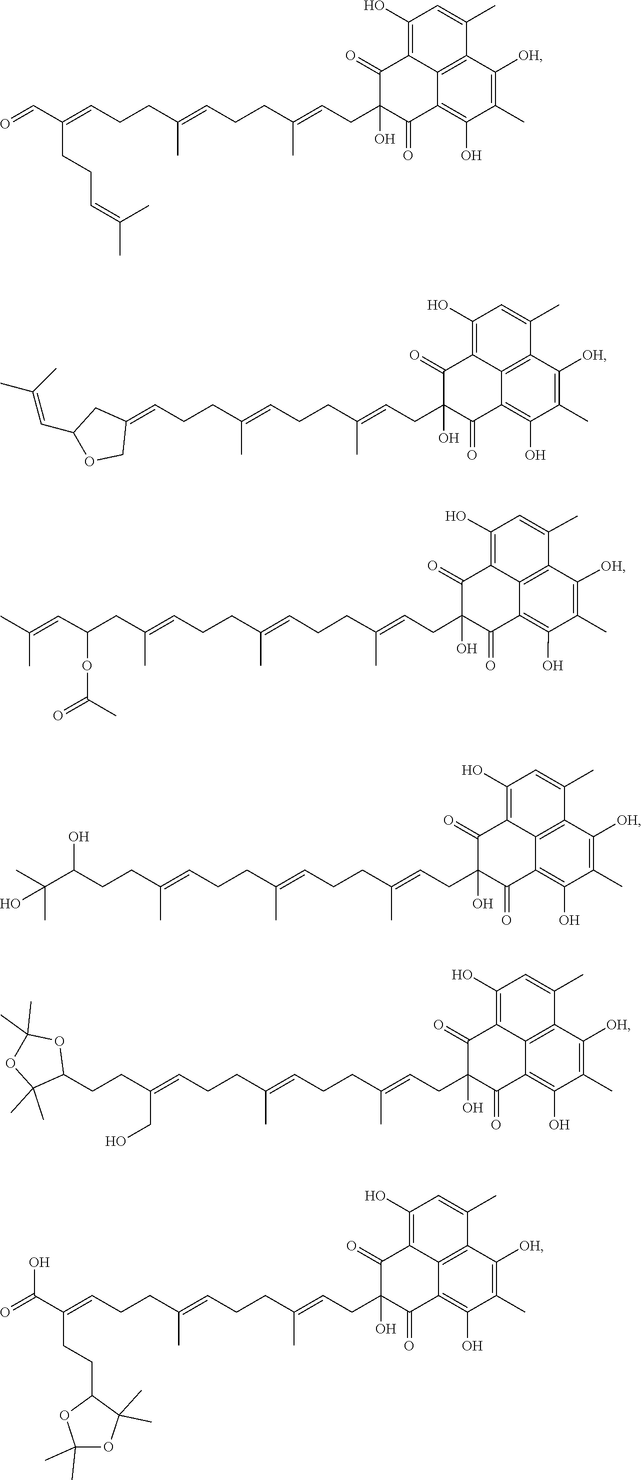 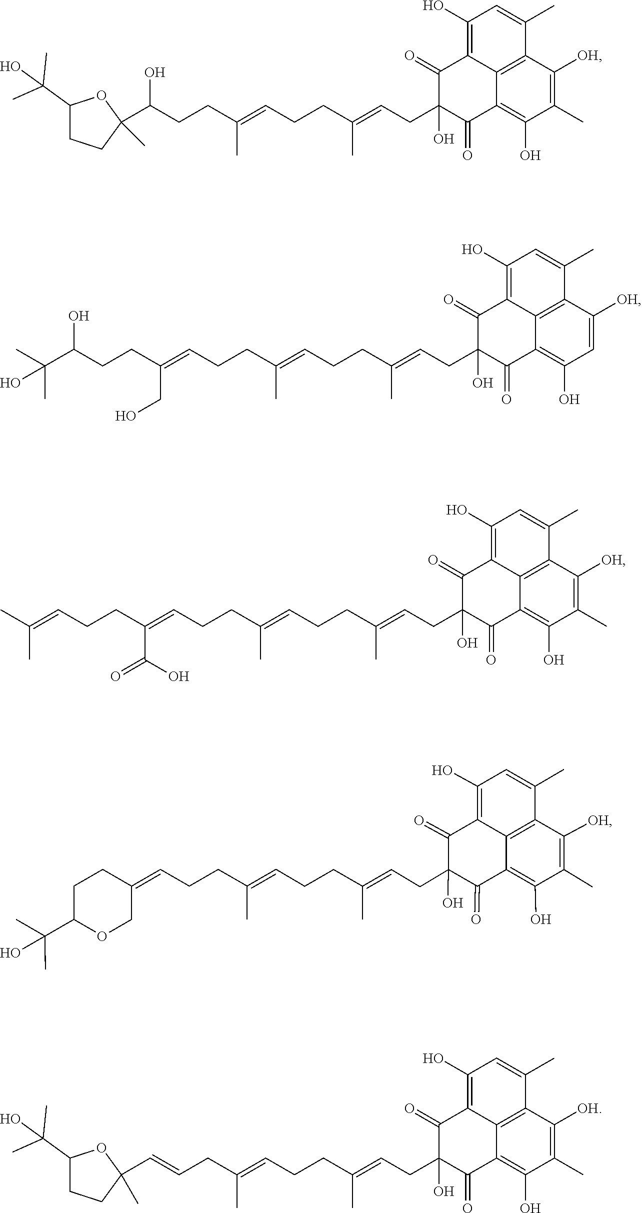 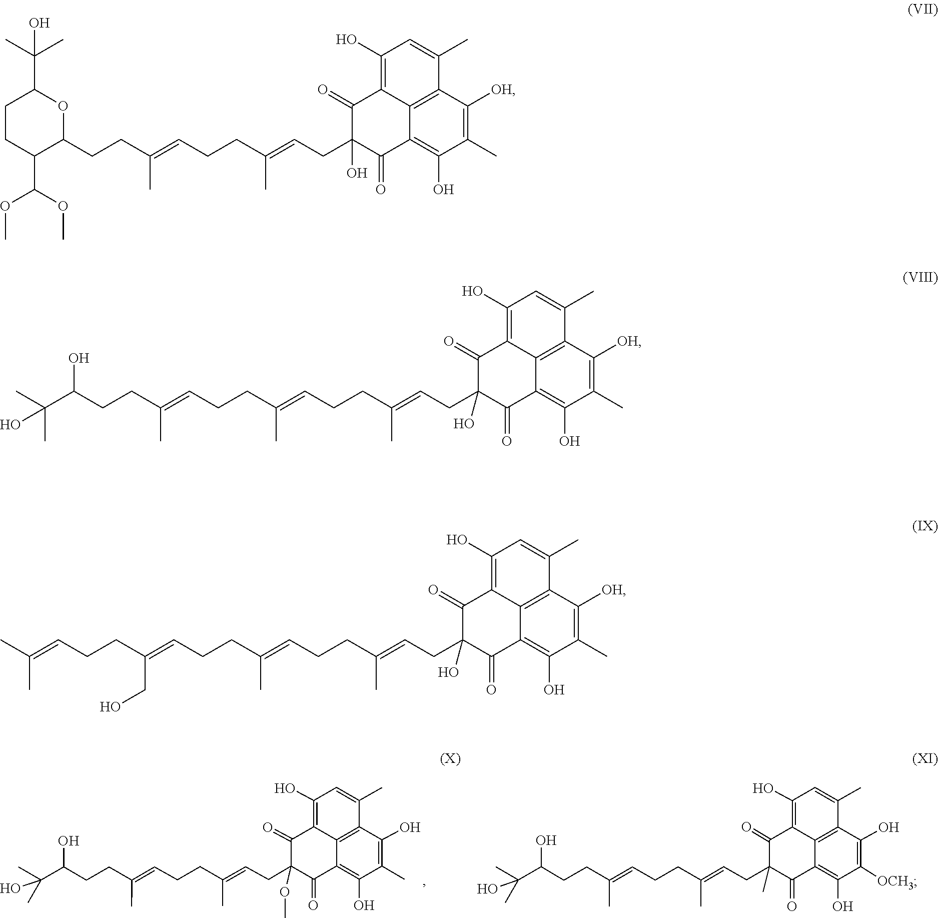 [SMILES: COC(OC)C1CCC(C(C)(C)O)OC1CC/C(C)=C/CC/C(C)=C/CC1(O)C(=O)c2c(O)cc(C)c3c(O)c(C)c(O)c(c23)C1=O ; C/C(=C\CC/C(C)=C/CC1(O)C(=O)c2c(O)cc(C)c3c(O)c(C)c(O)c(c23)C1=O)CC/C=C(\C)CCC(O)C(C)(C)O ; CC(C)=CCC/C(=C/CC/C(C)=C/CC/C(C)=C/CC1(O)C(=O)c2c(O)cc(C)c3c(O)c(C)c(O)c(c23)C1=O)CO ; COC1(C/C=C(\C)CC/C=C(\C)CCC(O)C(C)(C)O)C(=O)c2c(O)cc(C)c3c(O)c(C)c(O)c(c23)C1=O ; COc1c(O)c2c3c(c(O)cc(C)c3c1O)C(=O)C(C)(C/C=C(\C)CC/C=C(\C)CCC(O)C(C)(C)O)C2=O]
preferably, the stereoisomer is selected from the following structures:

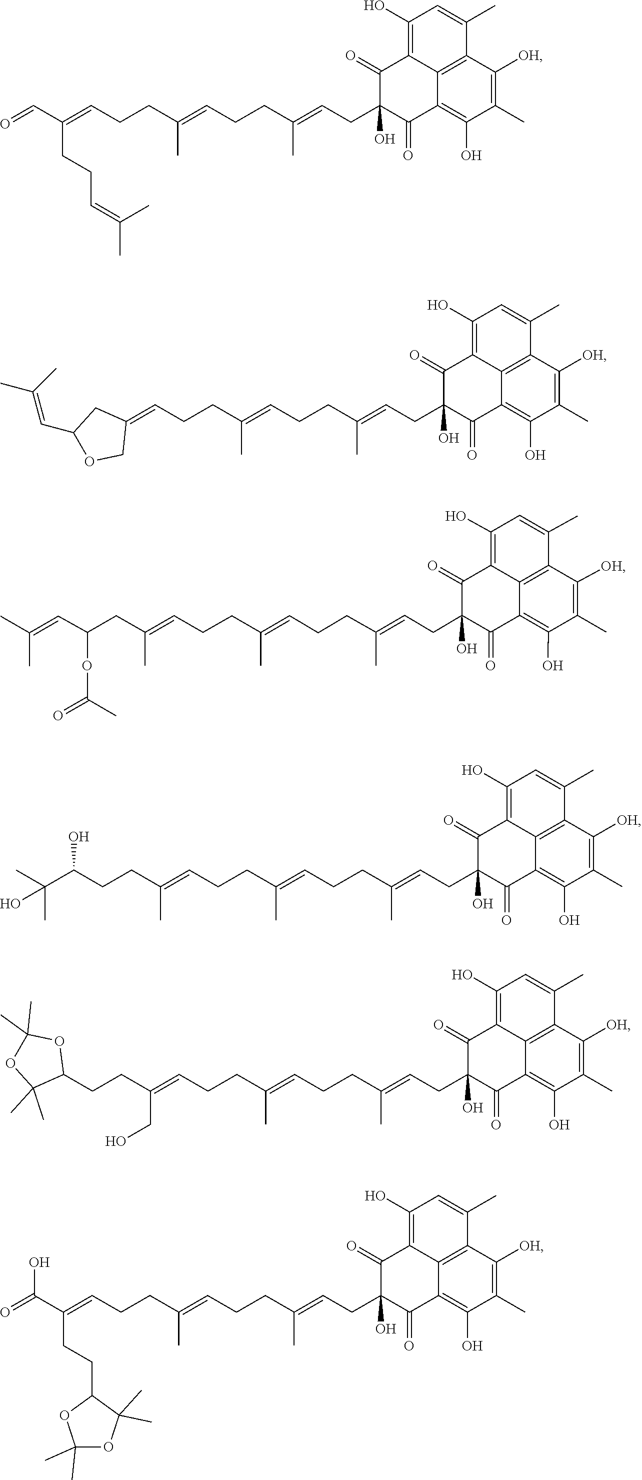

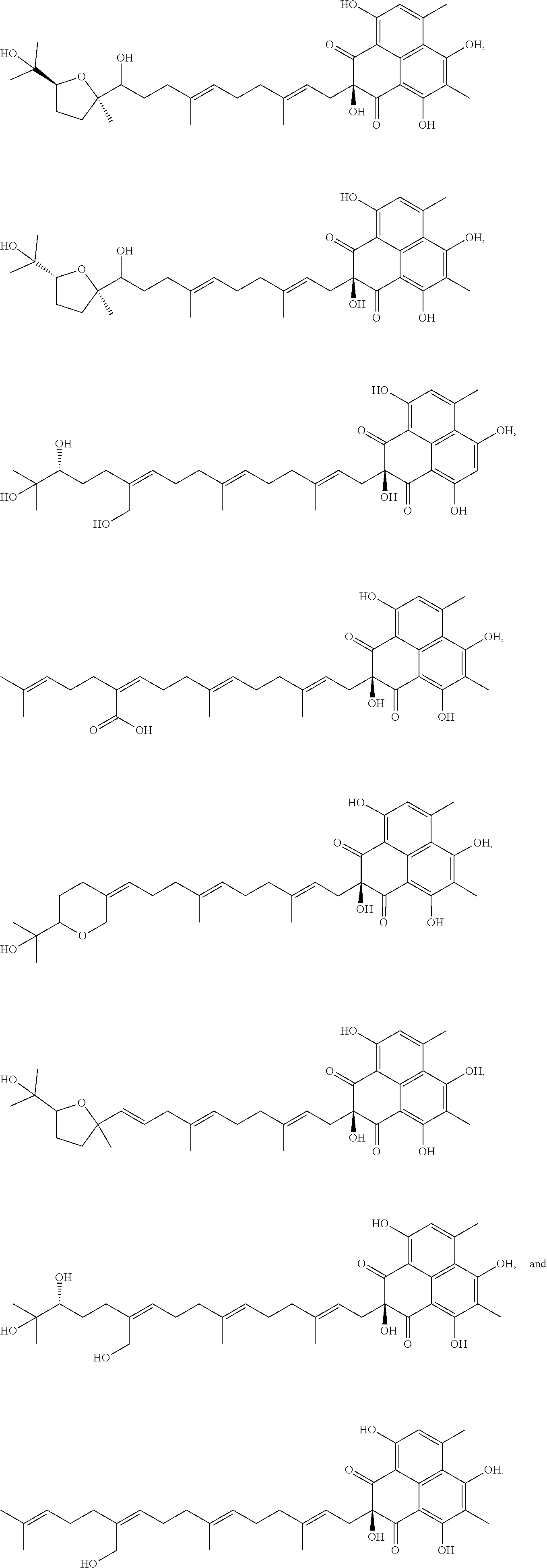

18. The use according to claim 17, wherein the PROTAC has the following structure:
SMI-L-E₃L (XII)
wherein SMI is a small molecule inhibitor moiety;
E3L is a ligand moiety of E3 ubiquitin ligase;
L is a linking bond or linking group between SMI and E₃L;
wherein E₃L is formed from the compound or the pharmaceutically acceptable salt, the stereoisomer, the ester, the prodrug, and the solvate thereof.

19. A PROTAC, wherein the PROTAC has the following structure:
SMI-L-E₃L (XII)
wherein SMI is a small molecule inhibitor moiety;
E₃L is a ligand moiety of E3 ubiquitin ligase;
L is a linking bond or linking group between SMI and E₃L;
wherein E₃L is formed from the compound according to claim 1 or a pharmaceutically acceptable salt, a stereoisomer, an ester, a prodrug, or a solvate thereof.

20. An aspergillus having an accession number of CGMCC No. 22467.
